(19) United States
(12) Patent Application Publication
(10) Pub. No.: US 2007/0178152 A1
(43) Pub. Date: Aug. 2, 2007

(54) Carboxyalkylcellulose esters for administration of poorly soluble pharmaceutically active agents

(76) Inventors: Michael C. Shelton, Kingsport, TN (US); Jessica D. Posey-Dowty, Kingsport, TN (US); Kevin J. Edgar, Kingsport, TN (US); Larry R. Lingerfelt JR., Blountville, TN (US); Sandra Klein, Ranstadt (DE); Shane K. Kirk, Church Hill, TN (US); Jennifer Dressman, Frankfurt am Main (DE)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/592,130

(22) Filed: Nov. 3, 2006

Related U.S. Application Data
(60) Provisional application No. 60/733,495, filed on Nov. 4, 2005.

(51) Int. Cl. A61K 31/717; A61K 9/20

(52) U.S. Cl. 424/464; 514/57

(57) ABSTRACT

Disclosed herein are pharmaceutical compositions comprising carboxyalkylcellulose esters for delivery of pharmaceutically active substances having low solubility in a medium such as water, an acidic aqueous buffer, a neutral aqueous buffer, or a basic aqueous buffer. Also disclosed are methods for making pharmaceutical compositions and methods of administering the compositions.

This application claims the benefit of priority of U.S. Provisional Patent Application No. 60/733,495 filed Nov. 4, 2005.

### FIELD OF THE INVENTION

Disclosed herein are pharmaceutical compositions comprising carboxyalkylcellulose esters for delivery of poorly soluble pharmaceutically active substances, e.g., having low solubility in a medium. Also disclosed are methods for making such pharmaceutical compositions and methods of administering the compositions.

### BACKGROUND

Solubility and the dissolution profile of a drug in media such as water, aqueous buffers (e.g. simulated gastric fluid (with or without pesin) and simulated intestinal fluid with or without pancreatin)) or in biorelevant media are parameters often used to assess the bioavailability of a drug substance. In vivo, a drug formulation enters a physiological environment where the drug dissolves and remains in solution. However, some drug substances fail to dissolve, or may precipitate over time (sometimes due to changes in pH). Thus, the pharmaceutical industry is interested in the fate of the drug formulation following introduction to the physiological environment.

Drug solubility has been a common limitation in the development of new drug formulations. More than a third of the drugs listed in the United States Pharmacopoeia are poorly soluble or are insoluble in water. (S. Pace et al, Pharm. Tech., pp. 116-132, March, 1999.) Additionally, it is well known that for many drugs the rate-limiting step for the absorption within the gastrointestinal tract is its dissolution. (D. Q. M. Craig et al., Int. J Pharm., Vol. 179, pp. 179-207, 1999.) To enhance the dissolution rate of poorly water soluble drug and to increase their bioavailability, several techniques have been developed, such as formulation strategies including the formation of solid dispersions. However, such formulations can often be thermodynamically unstable and/or cause undesired side effects.

Accordingly, there remains a need to develop compositions that improve the solubility and or dissolution of poorly water soluble pharmaceutically active agents.

### BRIEF DESCRIPTION OF THE DRAWINGS

FIG. 1 shows carbamazepine and carbamazepine solid dispersions dissolution profiles;

FIG. 2 shows glyburide and glyburide solid dispersions dissolution profiles;

FIG. 3 shows glyburide solid dispersions dissolution profiles;

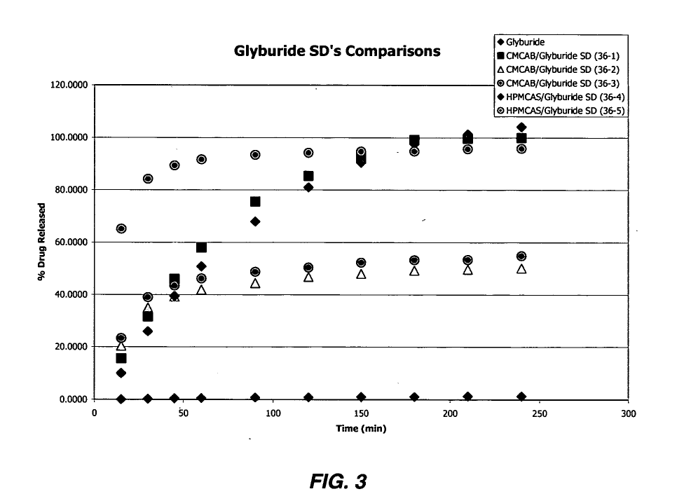

FIG. 4 shows a comparison of CMCAB/glyburide solid dispersion dissolution profiles (prepared by co-evaporation);

FIG. 5 shows a comparison of HPMCAS/glyburide solid dispersion profiles with those of CMCAB/glyburide solid dispersions;

FIG. 6 shows a comparison of HPMCAS/glyburide solid dispersion dissolution profiles with those of CMCAB/glyburide solid dispersions;

FIG. 7 shows a dissolution profile of griseofulvin solid dispersions;

FIG. 8 shows a comparison of CMCAB/griseofulvin solid dispersions dissolution profiles (% released);

FIG. 9 shows a comparison of CMCAB/griseofulvin solid dispersions dissolution profiles (mg released);

FIG. 10 shows a comparison of CMCAB/griseofulvin solid dispersion profiles with those of PVP/griseofulvin (% released);

FIG. 11 shows a comparison of CMCAB/griseofulvin solid dispersions dissolution profiles with those of PVP/griseofulvin solid dispersions (mg released);

FIG. 12 shows a comparison of CMCAB/griseofulvin solid dispersions dissolution profiles with those of HPMCAS/griseofulvin solid dispersions (% released);

FIG. 13 shows a comparison of CMCAB/griseofulvin solid dispersions dissolution profiles with those of HPMCAS/griseofulvin solid dispersions (mg released);

FIG. 14 shows griseofulvin, griseofulvin/CMCAB, and griseofulvin/CMCAB/surfactant solid dispersion dissolution profile comparisons; and

FIG. 15 shows the impact of TPGS on % crystallinity of ibuprofen/CMCAB solid dispersions (D-Optimal Mixture DOE Results).

### DETAILED DESCRIPTION

The present disclosure provides compositions comprising carboxyalkylcellulose esters for administering pharmaceutically active agents to a subject. One embodiment disclosed herein provides a pharmaceutical composition comprising:

at least one pharmaceutically active agent having low solubility in a medium, and

at least one carboxyalkylcellulose ester comprising an anhydroglucose repeat unit having the structure:

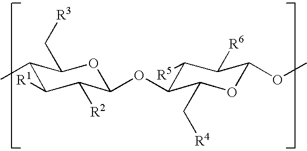

wherein:

R¹-R⁶ are each independently selected from —OH, —OC(O)(alkyl), and —O(CH₂)ₓC(O)OH, and pharmaceutically acceptable salts thereof, wherein x ranges from 1-3,

a degree of substitution per anhydroglucose of —OH ranges from 0.1 to 0.7,

a degree of substitution per anhydroglucose of —OC(O)(alkyl) ranges from 0.1 to 2.7, and

a degree of substitution per anhydroglucose of —O(CH₂)ₓC(O)OH ranges from 0.2 to 0.75.

“Degree of substitution” as used herein refers to a number of substituents per anhydroglucose. A theoretical maximum degree of substitution is 3 is assumed unless stated otherwise as in HS-CMC (high solids carboxymethylcellulose) esters or low molecular weight CMC esters, which can have a maximum degree of substitution per anhydroglucose unit of greater than 3.0.

In one embodiment, the pharmaceutically acceptable salts include pharmaceutically acceptable salts of —OH and —O(CH₂)ₓC(O)OH having the structure O⁻ A⁺ and —O(CH₂)ₓC(O)O⁻A⁺, respectively, wherein A⁺ is a counterion. Exemplary counterions include monovalent inorganic cations, such as lithium, sodium, potassium, rubidium, cesium, silver, divalent inorganic cations, such as magnesium, calcium, nickel, zinc, iron copper, or manganese, and ammonium and alkylammonium counterions. The counterion A⁺ need not necessarily be the same throughout the molecule and comprise a combination of differing counterions, as readily understood by one of ordinary skill in the art.

In one embodiment, the —OC(O)(alkyl) is chosen from —OC(O)(C₁-C₂₁ alkyl), such as —OC(O)(C₁-C₁₁ alkyl), —OC(O)(C₁-C₅ alkyl), or —OC(O)(C₁-C₃ alkyl). Alternatively, the —OC(O)(C₁-C₂₁ alkyl) can be referred to as a C₂-C₂₂ ester of a carboxyalkylcellulose ester.

In one embodiment, the carboxyalkylcellulose ester is chosen from carboxymethylcellulose esters. Exemplary carboxyalkylcellulose esters, include, but are not limited to carboxymethylcellulose acetate butyrate (CMCAB) (such as CMCAB-641-0.5 from Eastman Chemical Company), high solids CMCAB (HS-CMCAB), carboxymethylcellulose butyrate (CMCB), carboxymethylcellulose acetate propionate (CMCAP), high solids CMCAP (HS-CMCAP), carboxymethylcellulose propionate (CMCP), carboxymethylcellulose acetate (CMCA), carboxymethylcellulose acetate isobutryate (CMCAiB), carboxymethylcellulose isobutryate (CMCiB), carboxymethylcellulose acetate butyrate succinate, carboxymethylcellulose acetate butyrate maleate, carboxymethylcellulose acetate butyrate trimellitate.

In one embodiment, the at least one carboxyalkylcellulose ester is carboxymethylcellulose propionate having a degree of substitution per anhydroglucose of —OC(O)CH₂CH₃ ranging from 1.5 to 2.7.

In another embodiment, the at least one carboxyalkylcellulose ester is carboxymethylcellulose butyrate having a degree of substitution per anhydroglucose of —OC(O)CH₂CH₂CH₃ ranging from 1.5 to 2.7.

In yet another embodiment, the at least one carboxyalkylcellulose ester is carboxymethylcellulose acetate propionate having a degree of substitution per anhydroglucose of —OC(O)CH₃ ranging from 0.1 to 2.65 and a degree of substitution per anhydroglucose of —OC(O)CH₂CH₂H₃ ranging from 0.1 to 2.6.

In another embodiment, the at least one carboxyalkylcellulose ester is carboxymethylcellulose acetate butyrate having a degree of substitution per anhydroglucose of —OC(O)CH₃ ranging from 0.1 to 1.65 and a degree of substitution per anhydroglucose of —OC(O)CH₂CH₂H₃ ranging from 0.1 to 2.6.

In one embodiment, the medium is chosen from water, acidic aqueous buffers, neutral aqueous buffers, basic aqueous buffers, and natural and simulated bodily fluids, such as gastric fluid (with or without pepsin), or intestinal fluid (with or without pancreatin). In one embodiment the medium is chosen from pharmaceutically acceptable media.

In one embodiment, “low solubility”, “poorly soluble”, and “poorly water soluble” are indicated by the Biopharmaceutics Classification System (BCS). (Amidon, G. L.; Lennemas, H.; Shah, V. P.; Crison, J. R. “A Theoretical Basis for a Biopharmaceutic Drug Classification: The Correlation of in Vitro Drug Product Dissolution and in Vivo Bioavailability, Pharm. Res. 1995, 12(3), 413-420; Lennernas, H.; Abrahamsson, B. “The Use of Biopharmaceutic Classification of Drugs in Drug Discovery and Development: Current Status and Future Extension,” J. Pharmacy and Pharmacology, 2005, 57(3), 273-285; u, C. -Y.; Benet, L. Z., “Predicting Drug Disposition via Application of BCS: Transport/Absorption/Elimination Interplay and Development of a Biopharmaceutics Drug Disposition Classification System,” Pharm. Res. 2005, 22(1), 11-23; Dressman, J.; Butler, J.; Hempenstall, J.; Reppas, C. “The BCS: Where do we go from here?” Pharmaceutical Technology North America 2001, 25(7), 68-76.)

The bioavailability of a drug may be influenced by at least two factors: solubility and permeability of a drug or agent. The Biopharmaceutics Classification System (BCS), may be used to distinguish between classes of drugs based on the solubility and permeability of the drugs in vivo. The Biopharmaceutics Classification system provides four cases (or classes) of drugs. These cases (or classes) are defined as: Class 1, high solubility-high permeability drugs; Class 2, low solubility-high permeability drugs; Class 3, high solubility-low permeability drugs; and Class 4, low solubility-low permeability drugs.

In one embodiment, the at least one pharmaceutically active agent belongs to class 2, i.e., low solubility-high permeability drugs, according to the BCS.

In another embodiment, the at least one pharmaceutically active agent belongs to class 4, i.e., low solubility-low permeability drugs, according to the BCS.

In another embodiment, “low solubility”, “poorly soluble”, and “poorly water soluble” are defined as one that requires at least 10,000 mL of water to dissolve 1 g of the agent.

In one embodiment, the composition comprises a solid dispersion (also known as solid solution), i.e., the at least one pharmaceutically active agent is dispersed in a polymeric carrier. Without wishing to be bound by any theory, the polymeric carrier may disrupt the crystal structure of the drug, thereby reducing the crystal lattice energy. The energy required to dissolve the drug substance can be reduced, which may result in increased dissolution rates, and thus, the increased bioavailability of the pharmaceutically active agent.

In one embodiment, in the solid dispersion substantially all crystallinity of the pharmaceutically active agent is suppressed by the polymeric carrier. In one embodiment, the pharmaceutically active agent has a percent crystallinity of less than 20%, such as a percent crystallinity of less than 15%, less than 10%, less than 5%, less than 3%, or less than 1%. In one embodiment, the agent is amorphous. In one embodiment, no crystallinity is detected by x-ray in the solid dispersion containing the pharmaceutically active agent and the polymeric carrier.

In one embodiment, the polymeric carrier comprises the at least one carboxyalkylcellulose ester. In one embodiment, the carboxyalkylcellulose ester carrier can be blended with other conventional carriers, such as hydrophilic compounds or polymers. Exemplary carriers include physiologically inert compounds that are sometimes water soluble, e.g., polyethylene glycols, such as those disclosed in U.S. Pat. No. 6,197,787. Other-additives that may be combined with the at least one carboxyalkylcellulose ester include cellulose and its derivatives, such as microcrystalline cellulose (MCC), cellulose acetate butyrate (CAB), methylcellulose, polyethylene glycol, polypropylene glycol, copolymers of polyethylene glycol and polypropylene glycol, poly(vinylpyrrolidone), ethyl cellulose, hydroxyethyl cellulose, hydroxypropyl cellulose, carboxymethyl cellulose, carboxymethylethyl cellulose, starch, dextran, dextrin, chitosan, collagen, gelatin, bromelain, cellulose acetate, unplasticized cellulose acetate, plasticized cellulose acetate, reinforced cellulose acetate, cellulose acetate phthalate, cellulose acetate trimellitate, hydroxypropylmethylcellulose, hydroxypropylmethylcellulose phthalate, hydroxypropylmethylcellulose acetate succinate, hydroxypropylmethylcellulose acetate trimellitate, cellulose nitrate, cellulose diacetate, cellulose triacetate, agar acetate, amylose triacetate, beta glucan acetate, beta glucan triacetate, acetaldehyde dimethyl acetate, cellulose acetate ethyl carbamate, cellulose acetate phthalate, cellulose acetate methyl carbamate, cellulose acetate succinate, cellulose acetate dimethaminoacetate, cellulose acetate ethyl carbonate, cellulose acetate chloroacetate, cellulose acetate ethyl oxalate, cellulose acetate methyl sulfonate, cellulose acetate butyl sulfonate, cellulose acetate propionate, cellulose acetate p-toluene sulfonate, triacetate of locust gum bean, cellulose acetate with acetylated hydroxyethyl cellulose, hydroxylated ethylene-vinylacetate, cellulose acetate butyrate, polyalkenes, polyethers, polysulfones, polyethersulfones, polystyrenes, polyvinyl halides, polyvinyl esters and ethers, natural waxes and synthetic waxes.

Solid dispersions can be prepared by any method known in the art, including co-evaporation (spray drying, rotovapping, film casting, etc.), freeze drying (lyophilizing), co-precipitation (flake precipitation, powder precipitation, etc), melt blending, melt extrusion, co-grinding and roll mixing, and solvent-free processes.

Two exemplary methods include the fusion technique and the solvent technique. In the fusion technique, the drug is dissolved in a molten carrier (the carboxyalkylcellulose ester) and the mixture cooled to form a solid. In the solvent technique, drug and carrier are dissolved in a solvent, followed by removal of the solvent by evaporation, spray drying, freeze drying, or co-precipitation.

In one embodiment, the preparation of a solid dispersion composition comprises weighing out a polymeric carrier, such as a carboxyalkylcellulose ester (e.g. CMCAB, CMCAP, or CMCA), into a suitable vessel and an appropriate solvent is added to the vessel to dissolve the carboxyalkylcellulose ester. The drug is dissolved in a separate vessel in an appropriate solvent. Optionally, additives (e.g. surfactants, dispersants, etc) are dissolved in another vessel in an appropriate solvent. All the components of the desired solid dispersion are combined into a single vessel and thoroughly mixed. The solid dispersion is then generated by one of the following techniques: co-precipitation into a non-solvent (e.g. water), co-evaporation, spray drying, or freeze drying.

In one embodiment, co-precipitation is the general term used to describe the combination of a solution or mixture containing a polymeric carrier (e.g. a carboxyalkylcellulose ester) and a drug (e.g. a poorly soluble drug), and optionally one or more other additives dissolved in an organic solvent with an aqueous non-solvent to produce a precipitate that is an intimate mixture (i.e. solid dispersion) of the polymeric carrier, pharmaceutically active agent(s), and optionally one or more other additives from the organic solution/mixture. Two exemplary co-precipitation methods include flake precipitation and powder precipitation.

Flake precipitation, a process known to those skilled in the art of cellulose ester chemistry, can be accomplished by adding a thin stream of the polymer/drug/solvent mixture (i.e. dope) to the aqueous non-solvent. Then term flake precipitation comes from the typical appearance of the precipitate that is formed by the process. Those skilled in the art recognize that a number of process variables, including temperature, rate of addition, mixing rate, concentration of solids in the organic mixture, pH of the nonsolvent, organic solvent content in the precipitate mixture, hardening time, etc) can be adjusted to modify the physical nature (i.e. morphology, particle size, etc.) of the co-precipitate, the composition of the co-precipitate, and likely the dissolution profile of the solid dispersion.

Powder precipitation, a process known to those skilled in the art of cellulose ester chemistry, is accomplished by adding the aqueous non-solvent to the polymer/drug/solvent mixture (i.e. dope) with appropriate mixing and temperature. The term powder precipitation comes from the typical appearance of the precipitate that is formed by the process. Those skilled in the art recognize that a number of process variables, including temperature, rate of addition, mixing rate, concentration of solids in the organic mixture, pH of the nonsolvent, organic solvent content in the precipitate mixture, hardening time, etc) can be adjusted to modify the physical nature (i.e. morphology, particle size, etc.) of the co-evaporate, the composition of the co-evaporate, and likely the dissolution profile of the solid dispersion.

In one embodiment, co-evaporation is the general term used to describe the removal of solvent from a solution or mixture containing a polymeric carrier (e.g. a carboxyalkylcellulose ester) and a drug (e.g. a poorly soluble drug), and optionally one or more other additives dissolved in a volatile organic solvent or mixture of solvents to produce a precipitate that is an intimate mixture (i.e. solid dispersion) of the non-volatile components of the organic solution/mixture. The three co-evaporation methods used for the preparation of the compositions of this invention are rotary evaporation under reduced pressure, film formation (i.e. evaporation without mixing at atmospheric pressure), and spray drying.

Co-evaporation under reduced pressure, as recognized by those skilled in the, can be accomplished by a number of processes including but not limited to rotary evaporation and vacuum distillation.

In one embodiment, distillation at atmospheric pressure can be used to prepare solid dispersion compositions.

In one embodiment, the solid dispersion compositions can be prepared by co-evaporation by film formation. Co-evaporation by film formation can be accomplished by casting a film of the drug/carrier/additive/solvent mixture and allowing film formation to occur upon evaporation of the solvent at room temperature and atmospheric pressure. Those skilled in the art recognize that there are numerous process ways to accomplish film formation from lab scale methods to commercial scale methods and that changing various process parameters such as rate of evaporation, temperature, pressure, and humidity can impact the morphology of the film that is formed and change the performance (i.e. the release profiles) of the solid dispersions prepared via this process.

In one embodiment, the solid dispersion compositions can be prepared by spray drying. Those skilled in the art recognize that the selection of process parameters can be used to modify properties of the solid dispersions produced via this method.

In one embodiment, the compositions disclosed herein exhibit increased dissolution rates over that of the pharmaceutically active agent alone.

In one embodiment, the compositions disclosed herein exhibit a more sustained release profile than that of the pharmaceutically active agent alone. In one embodiment, “sustained release” refers to a sustained delivery (i.e., substantially continuous release) of the pharmaceutically active agent over time, such as a time of at least 4 h, e.g., a time ranging from 4-24 h, from 12-24 h, from 6-12 h, or even greater than 24 h, e.g., 1-5 days.

In one embodiment, the compositions disclosed herein exhibit a near zero-order release profile wherein the pharmaceutically active agent alone releases almost immediately. In one embodiment, “zero order release” is a type of sustained release indicated by a substantially linear plot of released pharmaceutically active agent over time, where “substantially linear” refers to a correlation coefficient (R) of at least 0.8, for a given time, such as a correlation coefficient of at least 0.9, or at least 0.95.

In one embodiment, in pharmaceutically acceptable media, such as aqueous media, the composition exhibits release of the pharmaceutically active agent at a target pH. In one embodiment, the target pH is at least 5, such as a pH of at least 6, or a pH of at least 6.5. In one embodiment, release of the pharmaceutically active agent is stopped or reduced to a very slow rate at gastric pH (e.g., approximately 1.2), whereas release as described herein occurs at intestinal pH (e.g., approximately 6.8).

In one embodiment, the polymeric carrier is water-swellable, i.e., the polymeric carrier can expand in volume upon exposure to water, such as at pH levels approaching neutral or basic values. In one embodiment, the carboxy(C₁-C₃)alkylcellulose esters can be modified to obtain desired response to water and pH. For example, increasing the acid number of the carboxyalkylcellulose ester may produce a polymer that is more sensitive to water and ultimately could produce a water-soluble carboxy(C₁-C₃)alkylcellulose ester. Alternatively, a carboxyalkylcellulose ester may be made more water soluble by performing at least one of: increasing the hydroxyl content on the backbone, replacing longer chain esters with shorter chain esters (e.g. replace butyryl content with acetyl content), and/or reducing the molecular weight of the cellulose.

In one embodiment, the at least one carboxyalkylcellulose ester has a low molecular weight, as described in WO 04/83253, the disclosure of which is incorporated herein by reference.

In one embodiment, changing the composition of the carboxyalkylcellulose ester may affect the way it interacts with solvent, drugs, pharmaceutical additives and other polymers. In one embodiment, selection of the appropriate polymer composition for a specific drug and optional additives can be aided by the use of solubility parameters to determine the “compatibility” of the polymeric carrier, the drug, and the optional additives.

“Pharmaceutically active agent” as used herein refers to a biologically active organics, biological compounds, and combinations and blends thereof, that can treat or prevent a condition or disease.

In one embodiment, the pharmaceutically active agent can be chosen from any suitable drug known in the art, such as those chosen from the classes of drugs including, for example, analgesics, anti-inflammatory agents, anthelmintics, anti-arrhythmic agents, antibiotics (including penicillins), anticoagulants, antidepressants, antidiabetic agents, antiepileptics, antihistamines, antihypertensive agents, antimuscarinic agents, antimycobacterial agents, antineoplastic agents, immunosuppressants, antithyroid agents, antiviral agents, anxiolytic sedatives (hypnotics and neuroleptics), astringents, beta-adrenoceptor blocking agents, blood products and substitutes, cardiac inotropic agents, contrast media, corticosteroids, cough suppressants (expectorants and mucolytics), diagnostic agents, diagnostic imaging agents, diuretics, dopaminergics (antiparkinsonian agents), haemostatics, immunological agents, lipid regulating agents, muscle relaxants, parasympathomimetics, parathyroid calcitonin and biphosphonates, prostaglandins, radio-pharmaceuticals, sex hormones (including steroids), anti-allergic agents, stimulants and anoretics, sympathomimetics, thyroid agents, vasodilators and xanthines.

Exemplary analgesics and anti-inflammatory agents include, but are not limited to, aloxiprin, auranofin, azapropazone, benorylate, diclofenac, diflunisal, etodolac, fenbufen, fenoprofen calcim, flurbiprofen, ibuprofen, indomethacin, ketoprofen, meclofenamic acid, mefenamic acid, nabumetone, naproxen, oxyphenbutazone, phenylbutazone, piroxicam, sulindac.

Exemplary anti-arrhythmic agents include amiodarone HCl, disopyramide, flecainide acetate, quinidine sulphate.

Exemplary anti-bacterial and anti-pneumocystic agents include, but are not limited to, atovaquone, azithromycin, benethamine penicillin, cinoxacin, ciprofloxacin HCl, clarithromycin, clofazimine, cloxacillin, demeclocycline, doxycycline, erythromycin, ethionamide, imipenem, nalidixic acid, nitrofurantoin, rifampin, rifampicin, spiramycin, sulphabenzamide, sulphadoxine, sulphamerazine, sulphacetamide, sulphadiazine, sulphafurazole, sulfamethizole, sulphamethoxazole, sulphapyridine, tetracycline, trimethoprim.

Exemplary anti-coagulants include, but are not limited to, dicoumarol, dipyridamole, nicoumalone, phenindione.

Exemplary anti-depressants include, but are not limited to, amoxapine, maprotiline HCl, mianserin HCL, nortriptyline HCl, trazodone HCL, trimipramine maleate.

Exemplary anti-diabetics include, but are not limited to, acetohexamide, chlorpropamide, gliclazide, glipizide, glyburide, tolazamide, tolbutamide, troglitazone.

Exemplary anti-epileptics include, but are not limited to, beclamide, carbamazepine, clonazepam, ethotoin, methoin, methsuximide, methylphenobarbitone, oxcarbazepine, paramethadione, phenacemide, phenobarbitone, phenytoin, phensuximide, primidone, sulthiame, valproic acid.

Exemplary anti-fungal agents include, but are not limited to, amphotericin, butoconazole nitrate, clotrimazole, econazole nitrate, fluconazole, flucytosine, griseofulvin, itraconazole, ketoconazole, miconazole, natamycin, nystatin, posaconazole, sulconazole nitrate, terbinafine HCl, terconazole, tioconazole, undecenoic acid.

Exemplary anti-gout agents include, but are not limited to, allopurinol, probenecid, sulphin-pyrazone.

Exemplary anti-helmintics include, but are not limited to, albendazole, bephenium hydroxynaphthoate, cambendazole, dichlorophen, ivermectin, mebendazole, niclosamide, oxamniquine, oxfendazole, oxantel embonate, praziquantel, pyrantel embonate, thiabendazole.

Exemplary anti-hypertensive agents include, but are not limited to, amlodipine, atenolol, benidipine, darodipine, dilitazem HCl, diazoxide, felodipine, guanabenz acetate, isradipine, minoxidil, nicardipine HCl, nifedipine, nimodipine, phenoxybenzamine HCl, prazosin HCl, reserpine, terazosin HCl, verapamil, verapamil HCl.

Exemplary anti-hypercholesterolemic, antihyperlipoproteinemic, and lipid regulating agents include, but are not limited to, atorvastatin, bezafibrate, clofibrate, etofibrate, fenofibrate, fluvastatin, gemfibrozil,lovastatin, pravastatin, probucol, simvastatin.

Exemplary anti-malarials include, but are not limited to, amodiaquine, chloroquine, chlorproguanil HCl, halofantrine HCl, mefloquine HCl, proguanil HCl, pyrimethamine, quinine sulphate.

Exemplary anti-migraine agents include, but are not limited to, dihydroergotamine mesylate, ergotamine tartrate, methysergide maleate, pizotifen maleate, sumatriptan succinate.

Exemplary anti-muscarinic agents include, but are not limited to, atropine, benzhexol HCl, biperiden, ethopropazine HCl, hyoscyamine, mepenzolate bromide, oxyphencylcimine HCl, tropicamide.

Exemplary anti-neoplastic agents and immunosuppressants include, but are not limited to,aminoglutethimide, amsacrine, azathioprine, busulphan, chlorambucil, cyclosporin, dacarbazine, docetaxel, estramustine, etoposide, irinotecan, lomustine, melphalan, mercaptopurine, methotrexate, mitomycin, mitotane, mitozantrone, paclitaxel, procarbazine HCl, rapamycin, tamoxifen, tamoxifen citrate, testolactone.

Exemplary anti-osteoporotic agents include, but are not limited to, raloxifene.

Exemplary anti-protazoal agents include, but are not limited to, benznidazole, clioquinol, decoquinate, diiodohydroxyquinoline, diloxanide furoate, dinitolmide, furzolidone, metronidazole, nimorazole, nitrofurazone, omidazole, tinidazole.

Exemplary anti-thyroid agents include, but are not limited to, carbimazole, propylthiouracil.

Exemplary anti-viral agents include, but are not limited to, acyclovir, nelfinavir, nevirapine, saquinavir.

Exemplary anxiolytic, sedatives, hypnotics and neuroleptics include, but are not limited to, alprazolam, amylobarbitone, barbitone, bentazepam, bromazepam, bromperidol, brotizolam, butobarbitone, carbromal, chlordiazepoxide, chlormethiazole, chlorpromazine, clobazam, clotiazepam, clozapine, diazepam, droperidol, ethinamate, flunanisone, flunitrazepam, fluopromazine, flupenthixol decanoate, fluphenazine decanoate, flurazepam, haloperidol, lorazepam, lormetazepam, medazepam, meprobamate, methaqualone, midazolam, nitrazepam, oxazepam, pentobarbitone, perphenazine pimozide, prochlorperazine, sulpiride, temazepam, thioridazine, triazolam, zopiclone.

Exemplary β-Blockers include, but are not limited to, acebutolol, alprenolol, atenolol, labetalol, metoprolol, nadolol, oxprenolol, pindolol, propranolol.

Exemplary cardiac inotropic agents include, but are not limited to, amrinone, digitoxin, digoxin, enoximone, lanatoside C, medigoxin.

Exemplary corticosteroids include, but are not limited to, beclomethasone, betamethasone, betamethasone-17-valerate, budesonide, cortisone acetate, desoxymethasone, dexamethasone, fludrocortisone acetate, flunisolide, flucortolone, fluticasone propionate, hyd rocortisone, hydrocortisone-21-hemisuccinate, methylprednisolone, prednisolone, prednisone, triamcinolone.

Exemplary diuretics include, but are not limited to, acetazolamide, amiloride, bendrofluazide, bumetanide, chlorothiazide, chlorthalidone, ethacrynic acid, frusemide, metolazone, spironolactone, triamterene.

Exemplary anti-parkinsonian agents include, but are not limited to, bromocriptine mesylate, lysuride maleate.

Exemplary gastro-intestinal agents include, but are not limited to, bisacodyl, cimetidine, cisapride, diphenoxylate HCl, domperidone, famotidine, loperamide, mesalazine, nizatidine, omeprazole, ondansetron HCL, ranitidine HCl, sulphasalazine.

Exemplary histamine H-Receptor antagonists include, but are not limited to, acrivastine, astemizole, cinnarizine, cyclizine, cyproheptadine HCl, dimenhydrinate, flunarizine HCl, loratadine, meclozine HCl, oxatomide, terrenadine.

Exemplary nitrates and other anti-anginal agents include, but are not limited to, amyl nitrate, glyceryl trinitrate, isosorbide dinitrate, isosorbide mononitrate, pentaerythritol tetranitrate.

Exemplary nutritional agents include, but are not limited to, betacarotene, vitamin A, vitamin B₂, vitamin D, vitamin E, vitamin K.

Exemplary opioid analgesics include, but are not limited to, codeine, dextropropyoxyphene, diamorphine, dihydrocodeine, meptazinol, methadone, morphine, nalbuphine, pentazocine.

Exemplary hormones include, but are not limited to, clomiphene citrate, danazol, ethinyloestradiol, medroxyprogesterone acetate, mestranol, methyltestosterone, norethisterone, norgestrel, oestradiol, conjugated oestrogens, progesterone, stanozolol, stiboestrol, testosterone, testosterone propionate, tibolone, thyroxine.

Exemplary stimulants include, but are not limited to, amphetamine, dexamphetamine, dexfenfluramine, fenfluramine, mazindol.

Exemplary diagnostics agents include, but are not limited to, iopanoic acid.

In one embodiment, the pharmaceutically active agent is chosen from phenytoin, carbamazepine, glyburide, and griseofulvin.

In one embodiment, the pharmaceutically active agent is chosen from those intended for oral administration. A description of these classes of drugs and a listing of species within each class can be found in Martindale, the Extra Pharmacopoeia, Thirty-fourth Edition, the Pharmaceutical Press, London, 2005, the disclosure of which is incorporated herein by reference. The drug substances are commercially available and/or can be prepared by techniques known in the art.

Exemplary nutraceuticals and dietary supplements can also be included, such as those disclosed in, for example, Roberts et al., Nutraceuticals: The Complete Encyclopedia of Supplements, Herbs, Vitamins, and Healing Foods (American Nutraceutical Association, 2001), which is specifically incorporated by reference. A nutraceutical or dietary supplement, also known as phytochemicals or functional foods, is generally any one of a class of dietary supplements, vitamins, minerals, herbs, or healing foods that have medical or pharmaceutical effects on the body. Exemplary nutraceuticals or dietary supplements include, but are not limited to, folic acid, fatty acids (e.g., DHA and ARA), fruit and vegetable extracts, vitamin and mineral supplements, phosphatidylserine, lipoic acid, melatonin, glucosamine/chondroitin, Aloe Vera, Guggul, glutamine, amino acids (e.g., iso-leucine, leucine, lysine, methionine, phenylanine, threonine, tryptophan, and valine), green tea, lycopene, whole foods, food additives, herbs, phytonutrients, antioxidants, flavonoid constituents of fruits, evening primrose oil, flax seeds, fish and marine animal oils, and probiotics. Nutraceuticals and dietary supplements also include bio-engineered foods genetically engineered to have a desired property, also known as pharmafoods.

In one embodiment, the pharmaceutical composition can include at least one pharmaceutically acceptable additive, binding agents, filling agents, lubricating agents, suspending agents, sweeteners, flavoring agents, preservatives, buffers, wetting agents, disintegrants, effervescent agents, surfactants, plasticizers, and other excipients. Such excipients are known in the art.

Exemplary binding agents include but not exclusively, carbohydrates, starches in native or treated form, lipids, waxes and fats.

Examples of filling agents are lactose monohydrate, lactose anhydrous, mannitol, and various starches; examples of binding agents are various celluloses and cross-linked polyvinylpyrrolidone, microcrystalline cellulose, such as Avicel® PH101 and Avicel® PH102, microcrystalline cellulose, and silicified microcrystalline cellulose (SMCC).

Suitable lubricants, including agents that act on the flowability of the powder to be compressed, are colloidal silicon dioxide, such as Aerosil® 200; talc, stearic acid, magnesium stearate, calcium stearate, and silica gel.

Examples of sweeteners are any natural or artificial sweetener, such as sucrose, xylitol, sodium saccharin, cyclamate, aspartame, and acsulfame. Examples of flavoring agents are Magnasweet® (trademark of MAFCO), bubble gum flavor, and fruit flavors, and the like.

Exemplary flavoring agents include, but are not limited to, Magnasweete® (trademark of MAFCO), bubble gum flavor, and fruit flavors.

Examples of preservatives are potassium sorbate, methylparaben, propylparaben, benzoic acid and its salts, other esters of parahydroxybenzoic acid such as butylparaben, alcohols such as ethyl or benzyl alcohol, phenolic compounds such as phenol, or quaternary compounds such as benzalkonium chloride.

Suitable diluents include pharmaceutically acceptable inert fillers, such as microcrystalline cellulose, lactose, dibasic calcium phosphate, saccharides, and/or mixtures of any of the foregoing. Examples of diluents include microcrystalline cellulose, such as Avicel® PH101 and Avicel® PH102; lactose such as lactose monohydrate, lactose anhydrous, and Pharmatose® DCL21; dibasic calcium phosphate such as Emcompress®; mannitol; starch; sorbitol; sucrose; and glucose.

Exemplary disintegrants include lightly crosslinked polyvinyl pyrrolidone, corn starch, potato starch, maize starch, and modified starches, croscarmellose sodium, cross-povidone, sodium starch glycolate, and mixtures thereof.

Exemplary effervescent agents are effervescent couples such as an organic acid and a carbonate or bicarbonate. Suitable organic acids include, for example, citric, tartaric, malic, fumaric, adipic, succinic, and alginic acids and anhydrides and acid salts. Suitable carbonates and bicarbonates include, for example, sodium carbonate, sodium bicarbonate, potassium carbonate, potassium bicarbonate, magnesium carbonate, sodium glycine carbonate, L-lysine carbonate, and arginine carbonate. Alternatively, only the acid component of the effervescent couple may be present.

Exemplary plasticizers include plasticizers that can be used in this invention include diethyl phthalate, triacetin, triethyl citrate, PEG 400, castor oil, propylene glycol, glycerin, low-molecular weight polyethylene glycols, surfactants, and organic acid esters, actetyltributyl citrate, acetyltriethyl citrate, benzyl benzoate, chlorobutanol, diacetylated monoglycerides, dibutyl sebacate, mineral oil and lanolin alcohols, petrolatum and lanolin alcohols.

Additional examples of plasticizers include carbohydrate and polyol esters such as but not limited to those described in U.S. Patent applications 2003/0171458 and 2005/0228084, for example glucose pentapropionate, α-glucose pentaacetate, β-glucose pentaacetate, α-glucose pentapropionate, β-glucose pentapropionate, α-glucose pentabutyrate and β-glucose pentabutyrate, xylitol acetate, xylitol propionate, xylitol butyrate, sorbitol acetate, sorbitol propionate, sorbitol butyrate, mannitol acetate, mannitol propionate, mannitol acetate.

Addition examples of plasticizers that may or may not be commonly used in pharmaceutical applications that might be used in the invention include Eastman DMP, Eastman DEP, Eastman DBP, butyl benzyl phthalate, dihexyl phthalate, Eastman DOP, C₆-C₁₀ straight-chain phthalate, C₇-C₁₁ 70% straight-chain phthalate, diisonoyl phthalate, diisodecyl phthalate, ditridecyl phthalate, Eastman DUP, Eastman TXIB, Eastman Triacetin, Eastman DOA, Dioctyl Azelate, Eastman TEG-EH, epoxidized tallate, Eastman TOTM, Eastman 425, triisooctyl trimellitate, triisononyl trimellitate, Eastman 168, Eastman EPZ, epoxidized soybean oil, Eastman PA-6.

Examples of quaternary ammonium compounds that might be used in this invention include di-N-alkyl(C₈-C₁₈ from coconut oil) dimethyl ammonium chloride, dimethyl dialkyl ammonium chloride, and poly(divinylbenzene-co-tirmethyl(vinylbenzyl)ammonium chloride).

Examples of other ingredients that might be included in the compositions include, amines and amino derivatives, amine-containing polymers including but not limited to chitosan, amide-containing polymers, including but not limited to chitin.

Other optional ingredients which may be included in the compositions of the present invention are antioxidants such as tocopherol, tocopherol acetate, ascorbyl palmitate, ascorbic acid, butylhydroxytoluene, butylhydroxyanisole and propyl gallate; pH stabilizers such as citric acid, tartaric acid, fumaric acid, acetic acid, glycine, arginine, lysine and potassium hydrogen phosphate; thickeners/suspending agents such as hydrogenated vegetable oils, beeswax, colloidal silicon dioxide, gums, celluloses, silicates, bentonite; flavouring agents such as cherry, lemon and aniseed flavors; sweeteners such as aspartame, saccharin and cyclamates; etc.

In one embodiment, the at least one additive is chosen from Vitamin E TPGS, sucrose acetate isobutyrate (SAIB), glucose pentapropionate (GPP), diethyl phthalate (DEP), triacetin, polyoxyethyenesorbitan monooleate (Tween 80) or sodium dodecylsulfate (SDS).

In another embodiment, the at least one additive is chosen Vitamin E TPGS, SAIB, glucose pentapropionate, DEP, triacetin, Tween 80 or sodium dodecylsulfate, lactose monohydrate, lactose anhydrous, mannitol, and various starches; examples of binding agents are various celluloses and cross-linked polyvinylpyrrolidone, microcrystalline cellulose, such as Avicel® PH101 and Avicel® PH102, microcrystalline cellulose, silicified microcrystalline cellulose (SMCC), colloidal silicon dioxide, such as Aerosil® 200; talc, stearic acid, magnesium stearate, calcium stearate, silica gel, sucrose, xylitol, sodium saccharin, cyclamate, aspartame, and acsulfame. Examples of flavoring agents are Magnasweet® (trademark of MAFCO), bubble gum flavor, fruit flavors, potassium sorbate, methylparaben, propylparaben, benzoic acid and its salts, other esters of parahydroxybenzoic acid such as butylparaben, alcohols such as ethyl or benzyl alcohol, phenolic compounds such as phenol, or quaternary compounds such as benzalkonium chloride, microcrystalline cellulose, lactose, dibasic calcium phosphate, saccharides, and/or mixtures of any of the foregoing. Examples of diluents include microcrystalline cellulose, such as Avicel® PH101 and Avicel® PH102, lactose such as lactose monohydrate, lactose anhydrous, and Pharmatose® DCL21, dibasic calcium phosphate such as Emcompress®, mannitol, starch, sorbitol, sucrose, glucose, lightly crosslinked polyvinyl pyrrolidone, corn starch, potato starch, maize starch, and modified starches, croscarmellose sodium, cross-povidone, sodium starch glycolate, Suitable organic acids include, for example, citric, tartaric, malic, fumaric, adipic, succinic, and alginic acids and anhydrides and acid salts. Suitable carbonates and bicarbonates include, for example, sodium carbonate, sodium bicarbonate, potassium carbonate, potassium bicarbonate, magnesium carbonate, sodium glycine carbonate, L-lysine carbonate, and arginine carbonate, α-cyclodextrin, β-cyclodextrin, and γ-cyclodextrin. More specifically examples of cyclodextrin derivatives include hydroxypropyl-α-cyclodextrin, hydroxypropyl-β-cyclodextrin, hydroxypropyl-γ-cyclodextrin, methyl-α-cyclodextrin, methyl-β-cyclodextrin, methyl-γ-cyclodextrin, ethyl-α-cyclodextrin, ethyl-β-cyclodextrin, ethyl-γ-cyclodextrin, hydroxybutenyl-α-cyclodextrin, hydroxybutenyl-β-cyclodextrin, hydroxybutenyl-γ-cyclodextrin, hydroxybutyl-α-cyclodextrin, hydroxybutyl-β-cyclodextrin, hydroxybutyl-γ-cyclodextrin, sulfobutyl-α-cyclodextrin, sulfobutyl-β-cyclodextrin, sulfobutyl-γ-cyclodextrin, sulfobutenyl-α-cyclodextrin, sulfobutenyl-β-cyclodextrin, sulfobutenyl-γ-cyclodextrin, organic esters of α-cyclodextrin, β-cyclodextrin, and γ-cyclodextrin where the organic esters can be an individual C₁-C₂₀ organic acid ester or mixture of C₁-C₂₀ acid esters and the cyclodextrin may be fully esterified or partially esterified, diethyl phthalate, triacetin, triethyl citrate, PEG 400, polyethylene glycol, castor oil, propylene glycol, glycerin, low-molecular weight polyethylene glycols, surfactants, and organic acid esters, actetyltributyl citrate, acetyltriethyl citrate, benzyl benzoate, chlorobutanol, diacetylated monoglycerides, dibutyl sebacate, mineral oil and lanolin alcohols, petrolatum and lanolin alcohols, glucose pentapropionate, α-glucose pentaacetate, β-glucose pentaacetate, α-glucose pentapropionate, β-glucose-pentapropionate, α-glucose pentabutyrate and β-glucose pentabutyrate, xylitol acetate, xylitol propionate, xylitol butyrate, sorbitol acetate, sorbitol propionate, sorbitol butyrate, mannitol acetate, mannitol propionate, mannitol acetate, di-N-alkyl(C₈-C₁₈ from coconut oil) dimethyl ammonium chloride, dimethyl dialkyl ammonium chloride, and poly(divinylbenzene-co-tirmethyl(vinylbenzyl)ammonium chloride), amines and amino derivatives, amine-containing polymers, chitosan, tocopherol, tocopherol acetate, ascorbyl palmitate, ascorbic acid, butylhydroxytoluene, butylhydroxyanisole and propyl gallate; pH stabilizers such as citric acid, tartaric acid, fumaric acid, acetic acid, glycine, arginine, lysine and potassium hydrogen phosphate; thickeners/suspending agents such as hydrogenated vegetable oils, beeswax, colloidal silicon dioxide, gums, celluloses, silicates, bentonite; flavoring agents such as cherry, lemon and aniseed flavors; sweeteners such as aspartame, saccharin and cyclamates.

The pharmaceutical composition can take a variety of forms, including, for example, those chosen from tablets, caplets, hard and soft gelatin capsules, non-gelatin-based capsules, powders, and sprinkles. The composition can be formulated into an oral dosage form. In another embodiment, the composition can be formulated for rectal, intravaginal, injectable, pulmonary, nasal, buccal, topical, local, intracisternal, intraperitoneal, ocular, aural, buccal spray, or nasal spray administration.

In one embodiment, when the pharmaceutical composition is in the form of a tablet, the composition is sufficiently compressible for tablet formation. In one embodiment, the composition can sustain a compression force of at least 10 psi for at least 10 seconds, such as a compression force of at least 100 psi for at least 10 seconds, such as a compression force of at least 1000 psi for at least 10 seconds.

The formulations disclosed herein can be made using at least one method chosen from spray drying, spray granulation, fluid bed granulation, high shear granulation, fluid bed drying, lyophilization, tableting, jet milling, pin milling, wet milling, rotogranulation, and spray coating.

In one embodiment, the composition comprises:

(a) at least one carboxyalkylcellulose ester in an amount ranging from 0.1 to 99 weight percent, based on the total weight (a) and (b) in said composition;

(b) the at least one pharmaceutically active agent in an amount ranging from 0.1 to 99 weight percent, based on the total weight (a) and (b) in said composition; and

(c) at least one additive chosen from plasticizers and flow aids in an amount ranging from 0 to 50 weight percent, based on the total weight of (a), (b), and (c) in the composition;

(d) an organic solvent, aqueous solvent, including but not limited to acetone, ethanol, ethyl acetate, dichloromethane, dimethyl sulfoxide, or water, or a solvent mixture;

wherein the total weight of (a) and (b) is about 5 to 95 weight percent of the total weight of (a), (b), (c), and (d).

In another embodiment, the composition comprises:

(a) about 0.1 to about 99 weight percent, based on the total weight (a) and (b) in said composition, of at least one carboxy(C₁-C₃)alkylcellulose ester as disclosed herein having an inherent viscosity of about 0.20 to 0.70 dL/g, as measured in a 60/40 (wt./wt.) solution of phenol/tetra-chloroethane at 25° C., a degree of substitution per anhydroglucose unit of carboxy(C₁-C₃)alkyl of greater than 0.2 to about 0.75, and a degree of substitution per anhydroglucose unit of C₂-C₂₀ esters of about 1.5 to about 2.70;

(b) about 0.1 to 99 weight percent, based on the total weight of (a) and (b) in said composition, of at least one pharmaceutically active agent having low solubility; and

(c) about 0 to about 50 weight percent, based on the total weight of (a), (b), and (c) in said composition, of at least one additive selected from plasticizers, flow aids, binding agents, filling agents, lubricating agents, suspending agents, sweeteners, flavoring agents, preservatives, buffers, wetting agents, disintegrants, effervescent agents, etc.

(d) at least one solvent chosen from aqueous and/or organic solvents, including but not limited to acetone, ethanol, ethyl acetate, dichloromethane, dimethyl sulfoxide, or water, or a solvent mixture;

wherein the total weight of (a) and (b) is about 5 to about 80 weight percent of the total weight of (a), (b), (c), and (d).

In one embodiment, the at least one carboxy(C₁-C₃)alkylcellulose ester is chosen from a C₂-C₄ ester of a carboxy(C₁-C₃)alkylcellulose ester.

In another embodiment, ingredient (a) in the compositions disclosed herein can comprise about 0.1 to about 99 weight percent, based on the total weight (a) and (b) in said composition, of a carboxymethylcellulose acetate butyrate, an inherent viscosity of about 0.20 to 0.70 dL/g, as measured in a 60/40 (wt./wt.) solution of phenol/tetra-chloroethane at 25° C., a degree of substitution per anhydroglucose unit of carboxy(C₁-C₃)alkyl of greater than 0.2 to about 0.75, and a degree of substitution per anhydroglucose unit of butyrate esters of about 1.5 to about 2.70, and a degree of substitution per anhydroglucose unit of acetate esters of about 0.1 to about 2.0, and a degree of substitution of hydroxyl groups of from about 0.01 to about 1.5.

In another embodiment, ingredient (a) in the compositions disclosed herein can comprise about 0.1 to about 99 weight percent, based on the total weight (a) and (b) in said composition, of a carboxymethylcellulose acetate propionate, an inherent viscosity of about 0.20 to 0.70 dL/g, as measured in a 60/40 (wt./wt.) solution of phenol/tetra-chloroethane at 25° C., a degree of substitution per anhydroglucose unit of carboxy(C₁-C₃)alkyl of greater than 0.2 to about 0.75, and a degree of substitution per anhydroglucose unit of propionate esters of about 1.5 to about 2.70, and a degree of substitution per anhydroglucose unit of acetate esters of about 0.1 to about 2.0, and a degree of substitution of hydroxyl groups of from about 0.01 to about 1.5.

In another embodiment, ingredient (a) in the compositions disclosed herein can comprise (a) about 0.1 to about 99 weight percent, based on the total weight (a) and (b) in said composition, of a carboxymethylcellulose acetate, an inherent viscosity of about 0.20 to 0.70 dL/g, as measured in a 60/40 (wt./wt.) solution of phenol/tetra-chloroethane at 25° C., a degree of substitution per anhydroglucose unit of carboxymethyl groups of greater than 0.2 to about 0.75, and a degree of substitution per anhydroglucose unit of acetate esters of about 1.5 to about 2.70, and a degree of substitution of hydroxyl groups of from about 0.01 to about 1.5.

In one embodiment, the composition comprises a solid dispersion comprising carboxymethylcellulose acetate butyrate (CMCAB), a BCS Class 2 drug. In another embodiment, the composition comprises a solid dispersion comprising carboxymethylcellulose acetate propionate (CMCAP) and at least one BCS Class 2 drug. In another embodiment, the composition comprises a solid dispersion comprising carboxymethylcellulose acetate (CMCA) and at least one BCS Class 2 drug. In another embodiment, the composition comprises a solid dispersion comprising carboxymethylcellulose acetate isobutyrate (CMCAiB), and at least one BCS Class 2 drug. In another embodiment, the composition comprises a solid dispersion comprising a C₂-C₂₀ alkyl acid (CMC C₂-C₂₀ Ester), and at least one BCS Class 2 drug. In another embodiment, the composition comprises a solid dispersion comprising at least one carboxymethylcellulose mixed ester of at least one C₂-C₂₀ alkyl acid (CMC C₂-C₂₀ mixed ester) and a BCS Class 2 drug.

In one embodiment, the composition comprises a solid dispersion comprising carboxymethylcellulose acetate butyrate (CMCAB), a BCS Class 4 drug. In another embodiment, the composition comprises a solid dispersion comprising carboxymethylcellulose acetate propionate (CMCAP) and at least one BCS Class 4 drug. In another embodiment, the composition comprises a solid dispersion comprising carboxymethylcellulose acetate (CMCA) and at least one BCS Class 4 drug. In another embodiment, the composition comprises a solid dispersion comprising carboxymethylcellulose acetate isobutyrate (CMCAiB), and at least one BCS Class 4 drug. In another embodiment, the composition comprises a solid dispersion comprising a C₂-C₂₀ alkyl acid (CMC C₂-C₂₀ Ester), and at least one BCS Class 4 drug. In another embodiment, the composition comprises a solid dispersion comprising at least one carboxymethylcellulose mixed ester of at least one C₂-C₂₀ alkyl acid (CMC C₂-C₂₀ mixed ester) and a BCS Class 4 drug.

Another embodiment disclosed herein provides a method of treating a mammal in need thereof with a pharmaceutical composition, comprising:
administering to the mammal in need of treatment the pharmaceutical composition comprising:
   a therapeutically effective amount of at least one pharmaceutically active agent having low solubility in a medium, and
   at least one carboxyalkylcellulose ester and pharmaceutically acceptable salts thereof comprising an anhydroglucose repeat unit having the structure: 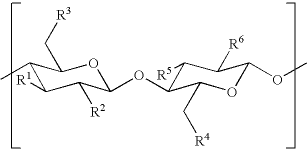
wherein:
R¹-R⁶ are each independently selected from —OH, —OC(O)(alkyl), and —O(CH₂)ₓC(O)OH, and pharmaceutically acceptable salts thereof, wherein x ranges from 1-3,
a degree of substitution per anhydroglucose of —OH ranges from 0.1 to 0.7,
a degree of substitution per anhydroglucose of —OC(O)(alkyl) ranges from 0.1 to 2.7, and
a degree of substitution per anhydroglucose of —O(CH₂)ₓC(O)OH ranges from 0.2 to 0.75.

In one embodiment, the terms “treatment” and its cognates (e.g., “therapeutic method”) refer to both therapeutic treatment and prophylactic/preventative measures. Those in need of treatment may include humans or animals already having a particular medical disease as well as those at risk for the disease (i.e., those who are likely to ultimately acquire the disorder). A therapeutic method results in the prevention or amelioration of symptoms or an otherwise desired biological outcome and may be evaluated by improved clinical signs, delayed onset of disease, reduced/elevated levels of lymphocytes and/or antibodies, etc.

Actual dosage levels of active ingredients in the pharmaceutical compositions described herein may be varied so as to obtain an amount of the active compound(s) that is effective to achieve the desired therapeutic response for a particular patient, compositions, and mode of administration. The terms “therapeutically effective dose” and “therapeutically effective amount” refer to that amount of a compound that results in prevention or amelioration of symptoms in a patient or a desired biological outcome, e.g., improved clinical signs, delayed onset of disease, reduced/elevated levels of lymphocytes and/or antibodies, etc. The effective amount can be determined as described herein. The selected dosage level will depend upon the activity of the particular compound, the route of administration, the severity of the condition being treated, and the condition and prior medical history of the patient being treated. However, it is within the skill of the art to start doses of the compound at levels lower than required to achieve the desired therapeutic effect and to gradually increase the dosage until the desired effect is achieved. In one embodiment, the data obtained from the assays can be used in formulating a range of dosage for use in humans.

Generally dosage levels of about 0.1 μg/kg to about 50 mg/kg, such as a level ranging from about 5 to about 20 mg of active compound per kilogram of body weight per day, can be administered topically, orally or intravenously to a mammalian patient. Other dosage levels range from about 1 μg/kg to about 20 mg/kg, from about 1 pg/kg to about 10 mg/kg, from about 1 μg/kg to about 1 mg/kg, from 10 μg/kg to 1 mg/kg, from 10 μg/kg to 100 μg/kg, from 100 μg to 1 mg/kg, and from about 500 μg/kg to about 5 mg/kg per day. If desired, the effective daily dose may be divided into multiple doses for purposes of administration, e.g., two to four separate doses per day. In one embodiment, the pharmaceutical composition can be administered once per day.

### EXAMPLES

Materials and Methods

Materials

Solvents:

Acetone (Burdick & Jackson # 010-4, Lot #'s CN784, CN755, and others)

Dichloromethane (Burdick & Jackson # 300-4, Lot #'s CN015 and others)

DMSO (Burdick & Jackson # 081-1; Lot #'s CN913 and others)

Water, Demineralized (used for co-precipitations)

Water (Deionized, Barnstead DiamondPure) (used to prepare dissolution media)

Tetrahydrofuran (Burdick & Jackson # 340-1, Lot #'s CL197 and others)

Acetonitrile (Burdick & Jackson # 015-4, Lot #'s CO997, CO106, and others)

Methanol (Burdick & Jackson # 230-4, Lot #'s CO680, CO357, CO914, and others

Ethanol (Aldrich # 493538-4L, Batch # 01950KC)

Isopropanol (Burdick & Jackson # 323-4, Lot #'s CN713, CL425, and others)

Reagents:

Potassium phosphate, monobasic (KH₂PO₄)(99%, ACS Reagent, Sigma # P0662, Lot # 064K0045)

Sodium hydroxide, 0.2 N (NaOH) (VWR # VW3220-1, Lot # 4194)

Triacetin (Eastman Chemical Company)

Diethyl phthalate (DEP) (Eastman Chemical Company)

Poly(ethylene glycol) (PEG-400) (Sigma, Cat. # P3265-1K, Batch # 054K0003)

Vitamin E TPGS, NF Grade (Eastman Chemical Company, Batch #'s 30035000 and 40008000)

Sucrose acetate isobutyrate (SAIB) (Eastman Chemical Company)

Polyoxyethylenesorbitan monooleate (Tween 80) (Sigma, Cat. # P8074, Lot # 87H0648)

Sodium dodecylsulfate, SDS (Sigma, Cat. # L6026-650G, Batch # 084K0001)

Polymers:

CMCAB-641-0.5 (Eastman Chemical Company) Lot # AG-0064B)

CMCAB (Eastman Chemical Company)

HPMCAS (CarboMer, Inc., Lot # BB-F4334)

PVP, K25 (Fluka, Cat. # 81399, Lot # 1124143)

Poly(ethylene glycol) (PEG 400) (Sigma-Aldrich # 20398-500G, Batch # 10528KA)

Polyethylene glycol (PEG 400) (Sigma-Aldrich # P3265-1KG, Batch # 054K0063)

Drugs:

Ibuprofen

Phenytoin (5,5-Diphenylhydantoin, ˜99%, Aldrich # D4007; Lot # 053K3668)

Carbamazepine (Sigma-Aldrich, Cat. # C4024-25G, Batch # 054K0646)

Glyburide (Sigma-Aldrich, Cat. # G-2539, Lot # 024K0701)

Griseofulvin (Sigma-Aldrich, Cat. # 64753-25G, Batch # 083K1219)

Azithromycin (LKT Laboratories, Cat. # A9834, Lot # 2393101)

Fenofibrate (Sigma-Aldrich, Cat. # F6020-25G, Batch # 064K1584)

Reference Standards:

Phenytoin RS (USP, Cat. # 1535008, Lot # 12B233)

Carbamazepine RS (USP, Cat. # 09300, Lot J)

Glyburide RS (USP, Cat. # 1295505, Lot # G1C347)

Griseofulvin RS (USP, Cat. # 29900, Lot I)

Azithromycin RS (USP, Cat. # 1046056, Lot # H0C212)

Capsules:

VCaps (Capsugel, size 0CS, Lot #630311)

Capsugel (0CS, Lot #624282)

Capsugel (00CS, Lot # 637785)

Equipment

All HPLC studies were performed on an Agilent 1100.

All dissolution studies were performed on a Varian VK7025 Dissolution Apparatus equipped with a Varian VK8000 Fraction Collector

Methods

Determination of Degree of Substitution by NMR: The ¹H NMR results were obtained using a JEOL Model GX-400 NMR spectrometer operated at 400 MHz. Sample tube size was 5 mm. The sample temperature was 80° C., the pulse delay 5 sec. and 64 scans were acquired for each experiment. Chemical shifts were reported in ppm from tetramethylsilane, with residual DMSO as an internal reference. The chemical shift of residual DMSO was set to 2.49 ppm.

For the carboxy(C₁-C₃)alkylcellulose esters, a GC method is used to determine acetyl, propionyl, and butyryl, rather than NMR, because the methylene of the carboxyl(C₁-C₃)alkyl group could not be easily separated from the ring protons of the cellulose backbone. The DS values were calculated by converting the acid number to percent carboxymethyl and using this along with the GC weight percents of acetyl, propionyl, and butyryl.

The acetyl, propionyl, and butyryl weight percents were determined by a hydrolysis GC method. In this method, about 1 g of ester was weighed into a weighing bottle and dried in a vacuum oven at 105° C. for at least 30 minutes. Then 0.500±0.001 g of sample was weighed into a 250 mL Erlenmeyer flask. To this flask was added 50 mL of a solution of 9.16 g isovaleric acid, 99%, in 2000 mL pyridine. This mixture was heated to reflux for about 10 minutes, after which 30 mL of isopropanolic potassium hydroxide solution is added. This mixture was heated at reflux for about 10 minutes. The mixture was allowed to cool with stirring for 20 minutes, and then 3 mL of concentrated hydrochloric acid is added. The mixture was stirred for 5 minutes, and then allowed to settle for 5 minutes. About 3 mL of solution is transferred to a centrifuge tube and centrifuged for about 5 minutes. The liquid was analyzed by GC (split injection and flame ionization detector) with a 25M·times·0.53 mm fused silica column with 1 μm FFAP phase.

The weight percent acyl was calculated as follows, where:

Ci=concentration of I (acyl group)

Fi=relative response factor for component I

Fs=relative response factor for isovaleric acid

Ai=area of component I

As=area of isovaleric acid

R=(grams of isovaleric acid)/(g sample)

Ci=((Fi*Ai)/Fs*As))*R*100

The GC method was used, along with NMR, to determine weight % acetyl, propionyl, and butyryl, and the method used is indicated.

The acid number of the carboxy(C₁-C₃)alkylcellulose esters was determined by titration as follows. An accurately weighed aliquot (0.5-1.0 g) of the carboxy(C₁-C₃)alkylcellulose ester was mixed with 50 mL of pyridine and stirred. To this mixture was added 40 mL of acetone followed by stirring. Finally, 20 mL of water was added and the mixture stirred again. This mixture was titrated with 0.1 N sodium hydroxide in water using a glass/combination electrode. A blank containing 50 mL of pyridine, 40 mL of acetone, and 20 mL of water was also titrated. The acid number was calculated as follows where:

Ep=mL NaOH solution to reach end point of sample

B=mL NaOH solution to reach end point of blank

N=normality of sodium hydroxide solution

Wt.=weight of carboxy(C₁-C₃)alkylcellulose ester titrated.
Acid Number (mg KOH/g sample)=((Ep−B)*N*56.1)/Wt.
IV Test Method

The inherent viscosity (IV) of the cellulose esters and carboxy(C₁-C₃)alkylcellulose esters described herein, except where indicated otherwise, was determined by measuring the flow time of a solution of known polymer concentration and the flow time of a solvent-blank in a capillary viscometer, and then calculating the IV. IV is defined by the following equation:
1(n)25° C.·0.50%=ln tₛtₒC

where:

(n)=Inherent Viscosity at 25° C. at a polymer concentration of 0.50 g/l 00 mL of solvent.

ln=Natural logarithm

tₛ=Sample flow time

tₒ=Solvent-blank flow time

C=Concentration of polymer in grams per 100 mL of solvent=0.50

Samples were prepared to a concentration of 0.50 g per 100 mL of solvent (60% phenol and 40% 1,1,2,2-tetrachloroethane, or “PM95,” by weight). The sample (0.25 g) was weighed into a culture tube containing a stir bar. 50.0 mL of 60% phenol and 40% 1,1,2,2-tetrachloroethane by weight (also described in the application as “PM95”) is added. The mixture was placed in a heater and heated with stirring (300 rpm) to 125° C. (7 minutes to reach the target temperature and 15 minute hold at 125° C.). The sample was allowed to cool to room temperature (25° C.) and was then filtered and placed in the viscometer (Model AVS 500—Schott America, Glass & Scientific Products, Inc., Yonkers, N.Y.). IV was calculated according to the equation above.

GPC Method for Molecular Weight Determination: The molecular weight distributions of cellulose ester and carboxy(C₁-C₃)alkylcellulose ester samples were determined by gel permeation chromatography (GPC) using one of two methods listed below.

Method 1, THF: The molecular weight distributions of cellulose ester samples indicated as being tested by GPC with THF as a solvent were determined at ambient temperature in Burdick and Jackson GPC-grade THF stabilized with BHT, at a flow rate of 1 mL/min. All other samples were determined using GPC with NMP as a solvent, as set forth in Method 2 below. Sample solutions were prepared by dissolution of about 50 mg of polymer in 10 mL of THF, to which 10 μL of toluene was added as a flow-rate marker. An autosampler was used to inject 50 μL of each solution onto a Polymer Laboratories PLgel™ column set including a 5 μm Guard, a Mixed-C™ and an Oligopore™ column in series. The eluting polymer was detected by differential refractometry, with the detector cell held at 30° C. The detector signal was recorded by a Polymer Laboratories Caliber™ data acquisition system, and the chromatograms were integrated with software developed at Eastman Chemical Company. A calibration curve was determined with a set of eighteen nearly monodisperse polystyrene standards with molecular weight from 266 to 3,200,000 g/mole and 1-phenylhexane at 162 g/mole. The molecular weight distributions and averages were reported either as equivalent polystyrene values, or as true molecular weights calculated by means of a universal calibration procedure with the following parameters:

Kₚₛ=0.0128 a_{PS}=0.712

K_{CE}=0.00757 a_{CE}=0.842

Method 2, NMP: The molecular weight distributions of all samples not otherwise indicated were determined by GPC with NMP as a solvent, as follows. The molecular weight distributions of cellulose ester samples were determined by gel permeation chromatography at 40° C. in Burdick and Jackson N-Methylpyrrolidone with 1% Baker glacial acetic acid by weight, at a flow rate of 0.8 mL/min. Sample solutions were prepared by dissolution of about 25 mg of polymer in 10 mL of NMP, to which 10 μL of toluene was added as a flow-rate marker. An autosampler was used to inject 20 μL of each solution onto a Polymer Laboratories PLgel™ column set including a 10 μm Guard, a Mixed-B™ column. The eluting polymer was detected by differential refractometry, with the detector cell held at 40° C. The detector signal was recorded by a Polymer Laboratories Caliber™ data acquisition system, and the chromatograms were integrated with software developed at Eastman Chemical Company. A calibration curve was determined with a set of eighteen nearly monodisperse polystyrene standards with molecular weight from 580 to 3,200,000 g/mole. The molecular weight distibutions and averages were reported as equivalent polystyrene values.

HPLC Determination of Ibuprofen in the presence of CMCAB or C-A-P:
Instrument: HP1100
Column: Zorbax SB—CN, 4.6×150 mm, 3.5 μm
Flow: 1.0 mL/min
Detection: UV at 225 nm
Injection volume: 10 μL
Temperature: not controlled

Mobile Phase:

| Min | Acetonitrile | 0.1% H3PO4 |
|---|---|---|
| 0 | 10 | 90 |
| 20 | 100 | 0 |
| 21 | 10 | 90 |

Post time: 8 min

Samples and standards were dissolved in Acetonitrile

HPLC Determination of Griseofulvin, Glyburide, Phenytoin, or Carbamazapine in the presence of CMCAB or C-A-P:
Instrument: HP1100
Column: Phenomenex Luna 3 μm phenyl-hexyl, 100×4.6 mm
Flow: 1.0 mL/min
Detection: UV at 230 nm for griseofulvin and glyburide
   UV at 220 nm for phenytoin
   UV at 240 nm for carbamazapine
Injection volume: 5 μL
Temperature: not controlled

Mobile Phase:

| Min | Acetonitrile | 0.1% H3PO4 |
|---|---|---|
| 0 | 30 | 70 |
| 9 | 95 | 5 |
| 15 | 95 | 5 |
| 15.5 | 30 | 70 |

Post time: 6 min
Samples and Standards dissolved in THF.

HPLC Methods for Evaluating Dissolution Aliquots:

Determination of Weight % Phenytoin, Carbamazepine, Glyburide, or Griseofulvin.

Mobile Phase: 55% acetonitrile/45% ammonium acetate buffer (2.6 g NH₄OAc/L H₂O with pH adjusted to pH˜5.25 with glacial acetic acid)

Column: Agilent Eclipse XDB-C8, 4.6 mm×150 mm×5 μm

Flow: 1.5 mL/min

Detection: UV 254 nm (UV 214 nm can also be used for phenytoin or carbamazepine, UV 291 nm can also be used for griseofulvin); typically five signals were selected from UV 214 nm, 222 nm, 254 nm, 287 nm, 291 nm, and/or 325 nm were collected for each sample.

Retention times were typically between 1.5 and 2.5 minutes.

Determination of Weight % Azithromycin

Mobile Phase: 60% acetonitrile/15% 0.002 N (NH₄)₂PO₄ buffer, pH 9.0/25% isopropanol

Column: Agilent Eclipse XDB-C8, 4.6 mm×150 mm×5 μm

Flow: 1.0 mL/min

Detection: Five signals selected from UV 210 nm, 214 nm, 220 nm, 230 nm, and 240 nm are collected for each sample.

Determination of % Crystallinity by X-ray.

All samples were run on a Scintag PAD V diffractometer using Cu K-alpha X-ray.

For each polymer or drug used in this study a neat sample was obtained. A known weight of each species was mixed with a know weight of corundum, Al₂O₃, diffraction standard. Each mixture was pelletized with a hydraulic press and the XRD pattern of the pellet was measured from 5 to 45 degree scattering angle. A diffraction response factor, R, was calculated for each species according to:
R=wc/ws*ls/lc
where wc id the weight fraction of corundum, ws is the weight fraction of the species of interest, lc is the net intensity of the major diffraction line of corrundum and is the net intensity of the major diffraction line of the drug or in the case of the polymers, the net intensity of the maximum of the amorphous scattering curve.

Samples were pelletized with a hydraulic press and the XRD pattern of the pellet was measured from 5 to 45 degree scattering angle. The net intensity of the maximum of the amorphous scatter from the polymer, lp, and the net intensity of the major diffraction line of the drug, ld, were determined from the resulting scattering curve. The wt % crystalline drug was calculated from:
% crystalline drug=(ld/Rd)/(ld/Rd+lp/Rp)×100
where Rd is the response factor for the drug and Rp is the response factor for the polymer.

Preparation of Glyburide Standard Curves

Glyburide reference standard from USP was dried as directed (106° C. for six hours), then approximately 20-25 mg of glyburide are added to a 25-mL volumetric flask and dissolved in DMSO or 55% acetonitrile/45% ammonium acetate, pH 5.25. The volume was diluted to 25 mL. A set of standard dilutions were prepared either using 10-mL volumetric flasks or Rainin automatic pipetmen.

Preparation of Dissolution Media

Simulated Intestinal Fluid, without pancreatin, pH 6.8 (SIFₛₚ, pH 6.8)—Added monobasic potassium phosphate (KH₂PO₄, 34 g) to a 4000-mL beaker. Added deionized/polished water (2000 mL) and mixed using a magnetic stir bar until the KH₂PO₄ is completely dissolved. Added 0.2 N sodium hydroxide (NaOH, 590 mL) and stirred. The pH was adjusted to pH 6.8±0.1 using 0.2 N NaOH. The sample was diluted with deionized/polished water to a final volume of 5000 mL.

The SIFsp media was heated to ˜45° C. in four 2000-mL Kimax bottles in an oven. The sample was degassed according to USP protocol by filtering through a 0.45 μm membrane filter (Pall, Supor-450, 0.45 μm, 90 mm, part # 60200, Lot # 43214) and stirring under vacuum for 5 minutes.

Simulated Gastric Fluid, without pepsin, pH 1.2 (SGFₛₚ, pH 1.2)—Dissolved 10.0 g of sodium chloride in 35.0 mL of hydrochloric acid and sufficient water to make 1000 mL. This test solution has a pH of about 1.2.

The SGFsp media was heated to ˜45° C. in four 2000-mL Kimax bottles in an oven. The sample was degassed according to USP protocol by filtering through a 0.45 μm membrane filter (Pall, Supor-450, 0.45 μm, 90 mm, part # 60200, Lot # 43214) and stirring under vacuum for 5 minutes.

Dissolution Conditions #1. Dissolution studies were performed on a Varian VK7025 Dissolution Apparatus equipped with a Varian VK8000 Fraction Collector using the following parameters: stir rate (50 rpm), sample size (5 mL), sample times (15 min, 30 min, 1 hr, 1 hr 30 min, 2 hr, 3 hr, 4 hr, 5 hr, 6 hr, 24 hr), bath temperature (37.3° C.), vessel temperature (37° C.), pump prime (60 sec), pump purge (60 sec), filter tips (10 μm).

Dissolution Conditions #2. Dissolution studies were performed on a Varian VK7025 Dissolution Apparatus equipped with a Varian VK8000 Fraction Collector using the following parameters: stir rate (75 rpm), sample size (5 mL), sample times (15 min, 30 min, 45 min, 1 hr, 1 hr 30 min, 2 hr, 2 hr 30 min, 3 hr, 3 hr 30 min, 4 hr), bath temperature (37.3° C.), vessel temperature (37° C.), pump prime (60 sec), pump purge (60 sec), filter tips (10 μm).

Buchi Model B-290/B295 Mini Spray Dryer Procedure.

System Description: The Buchi Model B-290/B295 Mini Spray Dryer is a lab-scale glass spray dryer with the capability to process flammable solvents. A closed-loop solvent recovery system with online oxygen monitoring allows safe processing of flammable solvents. Atomization is accomplished by a two-fluid nozzle. The feed material was supplied to the nozzle by a built-in peristaltic pump. The drying gas flow was co-current to atomization of the feed. Product is isolated from the gas stream by a cyclone separator. A bag filter was downstream of the cyclone to remove residual material from the exhaust gas stream.

Operating Procedure: The nitrogen supply valve was opened to provide an inert atmosphere to the dryer and the fan was switched on. The system was inspected to determine if there were any leaks in the glassware that would allow air into the system. Once the oxygen level was below 5%, the inlet temperature was set and the heater was switched on. The condenser temperature was set to the necessary temperature to allow removal of the solvent from the gas stream without freezing. The atomization gas was set to the desired flow by adjusting the flow meter. The feed material was inspected to ensure that the viscosity was suitable for adequate atomization and also to determine the need for filtration to remove insoluble materials. Once the desired inlet temperature was reached, the pump tubing was placed in the feed material and pump was switched on. The pump speed was set low (˜10%) and slowly increased if no problems were encountered. After the feed material was processed, clean solvent was pumped through the nozzle to prevent plugging. The heater was switched off and the fan was allowed to run to cool the unit. Once the unit was cool, the fan and atomization gas was switched off. The product collection container was removed and the product was transferred to a container. To maximize yield, the glassware was cleaned with a spatula and the product was collected and combined with that from the product container. Typical process conditions are given below.

| Conditions | 1 | 2 |
|---|---|---|
| Inlet Temp. (deg C.) | 55 | 55 |
| Outlet Temp. (deg C.) | 42 | 43 |
| Fan Setting (%) | 100 | 100 |
| Atomization Pressure | 30 | 30 |
| Feed Wt. (g) | 200 | 226.5 |
| Run Time (min.) | 42 | 42 |
| Pump Setting (%) | 17 | 20 |
| Feed Rate (g/min) | 4.76 | 5.39 |
| Yield | 6.5 | 24.3 |

### Example 1

This Example describes the preparation of solid dispersions by co-precipitation (flake method). “Co-precipitation” is the general term used to describe the combination of a solution or mixture containing a polymeric carrier (e.g. a carboxyalkylcellulose ester) and a pharmaceutically active agent, and optionally one or more other additives dissolved in an organic solvent with an aqueous non-solvent to produce a precipitate that is an intimate mixture (i.e. solid dispersion) of the non-volatile components of the organic solution/mixture. The two co-precipitation methods used for the preparation of the compositions of this invention are flake precipitation and powder precipitation.

Flake precipitation, a process known to those skilled in the art of cellulose ester chemistry, is accomplished by adding a thin stream of the polymer/drug/solvent mixture (i.e. dope) to the aqueous non-solvent. The term flake precipitation comes from the typical appearance of the precipitate that is formed by the process. Those skilled in the art would recognize that a number of process variables, including but not limited to temperature, rate of addition, mixing rate, concentration of solids in the organic mixture, pH of the nonsolvent, organic solvent content in the precipitate mixture, hardening time, etc) can be adjusted to modify the physical nature (i.e. morphology, particle size, etc.) of the co-precipitate, the composition of the co-precipitate, and likely the dissolution profile of the solid dispersion.

In the co-precipitation, flake method, an appropriate organic solvent or mixture of solvents (e.g. acetone, methylene chloride, ethanol, etc.) is added to a vessel (typically a glass bottle) containing the desired amount of the polymer carrier (in other examples, CMCAB, HPMCAS, PVP, or PEG), and the vessel is mixed (typically on a roller or by stirring) until a clear or at least mostly clear solution is obtained. The solids content of the mixture is adjusted by addition of solvent of solvent blend to produce a mixture with the desired viscosity. The drug substance is dissolved in an appropriate solvent or mixture of solvents (e.g. acetone, methylene chloride, ethanol, dimethyl sulfoxide, etc.) in a separate vessel. Optionally, one or more additives can be added to a third vessel and dissolved in an appropriate solvent. The polymer solution, the drug solution, and if included the additive solution are combined and thoroughly mixed. Alternately, the polymer and drug solids and optional additives, if desired or required, can be combined in a single vessel and then dissolved at the same time by the addition of an organic solvent or solvent mixture (This strategy is not always appropriate and should be tested on a case by case basis).

Co-precipitation is induced by pouring a small stream of the polymer/drug/additive solution into an excess of water, aqueous base, aqueous acid, or aqueous buffer solution with rapid mixing. Typically at least a ratio of 1:3 organic to aqueous solution is appropriate to induce flake precipitation, but a larger excess of aqueous solution is often appropriate depending on the percent solids in the system and the nature of the organic solvent in use. Once precipitation is complete, the sample if filtered on a coarse fritted funnel, dried overnight at 45° C. in a vacuum oven, and pulverized to a particle size of approximately 20 μm (typically less than 200 μm) in a cryogenic grinder. The samples are stored in a desiccator or vacuum desiccator until needed.

### Example 2

This Example describes the preparation of solid dispersions by co-precipitation (powder method). Powder precipitation, a process known to those skilled in the art of cellulose ester chemistry, is accomplished by adding the aqueous non-solvent to the polymer/drug/solvent mixture (i.e. dope) with appropriate mixing and temperature. Then term powder precipitation comes from the typical appearance of the precipitate that is formed by the process. Those skilled in the art would recognize that a number of process variables, including but not limited to temperature, rate of addition, mixing rate, concentration of solids in the organic mixture, pH of the nonsolvent, organic solvent content in the precipitate mixture, hardening time, etc) can be adjusted to modify the physical nature (i.e. morphology, particle size, etc.) of the co-precipitate, the composition of the co-precipitate, and likely the dissolution profile of the solid dispersion.

In the co-precipitation, powder method, an appropriate organic solvent or mixture of solvents (e.g. acetone, methylene chloride, ethanol, etc.) is added to a vessel (typically a glass bottle) containing the desired amount of the polymer carrier (in other examples, CMCAB, HPMCAS, PVP, or PEG), and the vessel is mixed (typically on a roller or by stirring) until a clear or at least mostly clear solution is obtained. The solids content is adjusted by addition of solvent of solvent blend to produce a mixture with the desired viscosity. The drug substance is dissolved in an appropriate solvent or mixture of solvents (e.g. acetone, methylene chloride, ethanol, dimethyl sulfoxide, etc.) in a separate vessel. Optionally, additional additive or additives are added to a third vessel and dissolved in an appropriate solvent. The polymer solution, the drug solution, and if included the additive solution are combined and thoroughly mixed. Alternately, the polymer and drug solids and optional additives, if desired or required, can be combined in a single vessel and then dissolved at the same time by the addition of an organic solvent or solvent mixture.

Co-precipitation is induced by slowly adding water, an aqueous base, an aqueous acid, or aqueous buffer solution to the polymer/drug/additive organic solution with rapid mixing. Typically at least a ratio of 1:3 organic to aqueous solution is appropriate to induce powder precipitation, but a larger excess of aqueous solution is often appropriate depending on the percent solids in the system and the nature of the organic solvent in use. Once precipitation is complete, the sample if filtered on a coarse fritted funnel, dried overnight at 45° C. in a vacuum oven, and pulverized to a particle size of approximately 20 μm (typically less than 200 μm) in a cryogenic grinder. The samples are stored in a desiccator or vacuum desiccator until needed.

### Example 3

This Example describes the preparation of solid dispersions by co-evaporation (reduced pressure method). “Co-evaporation” is the general term used to describe the removal of solvent from a solution or mixture containing a polymeric carrier (e.g. a carboxyalkylcellulose ester) and a drug as disclosed herein, and optionally one or more other additives dissolved in a volatile organic solvent or mixture of solvents to produce a precipitate that is an intimate mixture (i.e. solid dispersion) of the non-volatile components of the organic solution/mixture. The three co-evaporation methods used for the preparation of the compositions disclosed herein are rotary evaporation under reduced pressure, film formation (i.e. evaporation without mixing at atmospheric pressure), and spray drying.

In the co-evaporation, reduced pressure method, an appropriate organic solvent or mixture of solvents (e.g. acetone, methylene chloride, ethanol, etc.) is added to a vessel (typically a glass bottle) containing the desired amount of the polymer carrier (in other examples, CMCAB, HPMCAS, PVP, or PEG), and the vessel is mixed (typically on a roller or by stirring) until a clear or at least mostly clear solution is obtained. The solids content is adjusted by addition of solvent of solvent blend to produce a mixture with the desired viscosity. The drug substance is dissolved in an appropriate solvent or mixture of solvents (e.g. acetone, methylene chloride, ethanol, dimethyl sulfoxide, etc.) in a separate vessel. Optionally, one or more additives are added to a third vessel and dissolved in an appropriate solvent. The polymer solution, the drug solution, and if included the additive solution are combined and thoroughly mixed. Alternately, the polymer and drug solids and optional additives, if desired or required, can be combined in a single vessel and then dissolved at the same time by the addition of an organic solvent or solvent mixture.

Co-evaporation is induced by removing the solvent from the system using a rotary evaporator, typically at 50° C. Upon completion of evaporation the sample is placed on a high vacuum line over night to remove as much residual solvent as possible. The sample is removed from the round-bottomed flask by with a spatula. The sample is then dried overnight at 45° C. in a vacuum oven and then pulverized to a particle size of approximately 20 μm in a cryogenic grinder. The samples are stored in desiccator or vacuum desiccator until needed.

### Example 4

This Example describes the preparation of solid dispersions by co-evaporation via the film formation method. An appropriate organic solvent or mixture of solvents (e.g. acetone, methylene chloride, ethanol, etc.) is added to a vessel (typically a glass bottle) containing the desired amount of the polymer carrier (in other examples, CMCAB, HPMCAS, PVP, or PEG), and the vessel is mixed (typically on a roller or by stirring) until a clear or at least mostly clear solution is obtained. The solids content was adjusted by addition of solvent of solvent blend to produce a mixture with the desired viscosity. The drug is dissolved in an appropriate solvent or mixture of solvents (e.g. acetone, methylene chloride, ethanol, dimethyl sulfoxide, etc.) in a separate vessel. Optionally, one or more additives are added to a third vessel and dissolved in an appropriate solvent. The polymer solution, the drug solution, and if included the additive solution are combined-and thoroughly mixed. Alternately, the polymer and drug solids and optional additives, if desired or required, can be combined in a single vessel and then dissolved at the same time by the addition of an organic solvent or solvent mixture (This strategy is not always appropriate and should be tested on a case by case basis).

Co-evaporation is induced by removing the solvent from the system by pouring the sample into an appropriate vessel or onto a glass or metal sheet and allowing the solvent to slowly evaporate and a film to be formed. Those skilled in the art would recognize that a number of parameters can be controlled to influence the properties of the film formed. Typically in these examples the samples are poured into an evaporation dish and allowed to stand overnight while covered with a paper towel. Upon completion of film formation the sample is removed from the dish with a spatula. The sample is then dried overnight at 45° C. in a vacuum oven and then pulverized to a particle size of approximately 20 μm (typically less than 200 μm) in a cryogenic grinder. The samples are stored in a desiccator or vacuum desiccator until needed.

### Examples 5-18

These Examples describe the preparation of ibuprofen/CMCAB solid dispersions and ibuprofen/CMCAB/DEP solid dispersions. Solid dispersions containing ibuprofen (IB), carboxymethylcellulose acetate butyrate (CMCAB), and optionally diethlpthalate (DEP) were prepared by the co-precipitation, flake method of Example 1. Specific details of the preparation are listed in Table 1 below.

TABLE 1

| | CMCAB | IB | DEP | CMCAB | | DEP | % Crystalline | Wt. % | Wt. % | Wt % |
|---|---|---|---|---|---|---|---|---|---|---|
| Example # | (g)¹ | (g)¹ | (g)¹ | % | IB % | % | IB² | IB³ | CMCAB⁴ | DEP³ |
| 5 | 4.37 | 1.66 | 0.4 | 67.96 | 25.82 | 6.22 | 6 | 24.4 | 68.9 | 6.7 |
| 6 | 5.94 | 0.06 | 0 | 99.00 | 1.00 | 0.00 | 0 | 1.3 | 98.6 | 0.1 |
| 7 | 5.22 | 0.49 | 0.31 | 86.71 | 8.14 | 5.15 | 0 | 7.3 | 87.3 | 5.4 |
| 8 | 5.07 | 0.93 | 0 | 84.50 | 15.50 | 0.00 | 2.2 | 14.1 | 85.7 | 0.2 |
| 9 | 5.11 | 1 | 0.31 | 79.60 | 15.58 | 4.83 | 4.4 | 16.8 | 78.8 | 4.4 |
| 10 | 5.36 | 0.09 | 0.61 | 88.45 | 1.49 | 10.07 | 0 | 1.6 | 89.2 | 9.2 |
| 11 | 5.94 | 0.09 | 0 | 98.51 | 1.49 | 0.00 | 0 | 1.2 | 98.7 | 0.1 |
| 12 | 3.67 | 1.8 | 0.71 | 59.39 | 29.13 | 11.49 | 6.7 | 27 | 62.4 | 10.6 |
| 13 | 4.47 | 0.93 | 0.6 | 74.50 | 15.50 | 10.00 | 3.1 | 14.6 | 77.8 | 7.6 |
| 14 | 4.42 | 1.77 | 0 | 71.41 | 28.59 | 0.00 | 7.5 | 28.8 | 71.2 | 0 |
| 15 | 4.22 | 1.77 | 0 | 70.45 | 29.55 | 0.00 | 6.9 | 27.7 | 72.3 | 0 |
| 16 | 3.67 | 0.07 | 0.8 | 80.84 | 1.54 | 17.62 | 0 | 1.9 | 85.8 | 12.3 |
| 17 | 3.82 | 1.8 | 0.6 | 61.41 | 28.94 | 9.65 | 8.8 | 29.8 | 69.1 | 1.1 |
| 18 | 4.61 | 1.4 | 0.15 | 74.84 | 22.73 | 2.44 | 4.9 | 21.6 | 76.4 | 2 |

¹Amount added prior to co-precipitation
²Amount in solid dispersions, determined by x-ray
³Amount in solid dispersions, determined by HPLC
⁴Amount is solid dispersions, determined by difference calculations (Wt. % CMCAB = 100 − Wt. % IB − Wt. % DEP)

### Examples 19-32

These Examples describe the preparation of ibuprofen/CMCAB solid dispersions and ibuprofen/CMCAB/triacetin solid dispersions. Solid dispersions of containing ibuprofen (IB), carboxymethylcellulose acetate butyrate (CMCAB), and y triacetin were prepared by the co-precipitation, flake method of Example 1. Specific details of the preparation are listed in Table 2 below.

TABLE 2

| | CMCAB | IB | Triacetin | CMCAB | | Triacetin | % Crystalline | Wt. % | Wt. % | Wt % |
|---|---|---|---|---|---|---|---|---|---|---|
| Example # | (g)¹ | (g)¹ | (g)¹ | % | IB % | % | IB² | IB³ | CMCAB⁴ | Triacetin³ |
| 19 | 4.3 | 1.39 | 0.45 | 70.03 | 22.64 | 7.33 | 5.4 | 23.5 | 76.5 | 0 |
| 20 | 6.08 | 0.11 | 0 | 98.22 | 1.78 | 0.00 | 0 | 2.3 | 97.7 | 0 |
| 21 | 5.205 | 0.495 | 0.3 | 86.75 | 8.25 | 5.00 | 0 | 8.1 | 90.3 | 1.6 |
| 22 | 5.31 | 0.93 | 0 | 85.10 | 14.90 | 0.00 | 1.2 | 13.6 | 86.4 | 0 |
| 23 | 4.81 | 0.93 | 0.35 | 78.98 | 15.27 | 5.75 | 1.8 | 14.9 | 85.1 | 0 |
| 24 | 5.42 | 0.06 | 0.8 | 86.31 | 0.96 | 12.74 | 0 | 2.2 | 95 | 2.8 |
| 25 | 5.95 | 0.06 | 0 | 99.00 | 1.00 | 0.00 | 0 | 1.3 | 98.7 | 0 |
| 26 | 3.6 | 1.85 | 0.67 | 58.82 | 30.23 | 10.95 | 11 | 32.8 | 66.3 | 0.9 |
| 27 | 4.55 | 0.93 | 0.65 | 74.23 | 15.17 | 10.60 | 3.9 | 16.5 | 82.5 | 1 |
| 28 | 4.74 | 1.8 | 0 | 72.48 | 27.52 | 0.00 | 8.3 | 26.5 | 73.5 | 0 |
| 29 | 4.76 | 1.79 | 0 | 72.67 | 27.33 | 0.00 | 8.7 | 24.3 | 75.7 | 0 |
| 30 | 5.65 | 0.19 | 0.69 | 86.52 | 2.91 | 10.57 | 0 | 3.8 | 93.7 | 2.5 |
| 31 | 3.65 | 1.83 | 0.84 | 57.75 | 28.96 | 13.29 | 8.2 | 31.9 | 66.8 | 1.3 |
| 32 | 4.47 | 1.37 | 0.15 | 74.62 | 22.87 | 2.50 | 5.9 | 23.7 | 76.1 | 0.2 |

¹Amount added prior to co-precipitation
²Amount in solid dispersions, determined by x-ray
³Amount in solid dispersions, determined by HPLC
⁴Amount is solid dispersions, determined by difference calculations (Wt. % CMCAB = 100 − Wt. % IB − Wt. % Triacetin)

### Examples 33-45

These Examples describe the preparation of ibuprofen/CMCAB solid dispersions and ibuprofen/CMCAB/SAIB solid dispersions. Solid dispersions of containing ibuprofen (IB), carboxymethylcellulose acetate butyrate (CMCAB), and optionally sucrose acetate isobutyrate (SAIB) were prepared by the co-precipitation, flake method of Example 1. Specific details of the preparation are listed in Table 3 below.

TABLE 3

| | CMCAB | IB | SAIB | CMCAB | | SAIB | % Crystalline | Wt. % | Wt. % | Wt % |
|---|---|---|---|---|---|---|---|---|---|---|
| Example # | (g)¹ | (g)¹ | (g)¹ | % | IB % | % | IB² | IB³ | CMCAB⁴ | SAIB⁵ |
| 33 | 4.18 | 1.39 | 0.45 | 69.44 | 23.09 | 7.48 | 4.4 | 23 | 77 | 0 |
| 34 | 5.94 | 0.09 | 0 | 98.51 | 1.49 | 0.00 | 0 | 1.7 | 98.3 | 0 |
| 35 | 5.2 | 0.495 | 0.3 | 86.74 | 8.26 | 5.00 | 0 | 8.1 | 91.9 | 0 |
| 36 | 5.16 | 0.93 | 0 | 84.73 | 15.27 | 0.00 | 1.7 | 14.7 | 85.3 | 0 |
| 37 | 4.78 | 0.93 | 0.38 | 78.49 | 15.27 | 6.24 | 2.4 | 15 | 85 | 0 |
| 38 | 5.54 | 0.06 | 0.6 | 89.35 | 0.97 | 9.68 | 0 | 1.4 | 98.6 | 0 |
| 39 | 5.99 | 0.06 | 0 | 99.01 | 0.99 | 0.00 | 0 | 3.6 | 96.4 | 0 |
| 40 | 4.47 | 0.99 | 0.713 | 72.41 | 16.04 | 11.55 | 2.8 | 15.2 | 84.8 | 0 |
| 41 | 4.28 | 1.82 | 0 | 70.16 | 29.84 | 0.00 | 8.2 | 28.3 | 71.7 | 0 |
| 42 | 4.22 | 1.83 | 0 | 69.75 | 30.25 | 0.00 | 7.5 | 30.6 | 69.4 | 0 |
| 43 | 5.37 | 0.09 | 0.59 | 88.76 | 1.49 | 9.75 | 0 | 2.1 | 97.9 | 0 |
| 44 | 3.64 | 1.8 | 0.6 | 60.26 | 29.80 | 9.93 | 8.1 | 27.7 | 72.3 | 0 |
| 45 | 4.48 | 1.36 | 0.15 | 74.79 | 22.70 | 2.50 | 6.5 | 22.8 | 77.2 | 0 |

¹Amount added prior to co-precipitation
²Amount in solid dispersions, determined by x-ray
³Amount in solid dispersions, determined by HPLC
⁴Amount is solid dispersions, determined by difference calculations (Wt. % CMCAB = 100 − Wt. % IB − Wt. % SAIB)
⁵SAIB was not detectable by the LC method utilized

### Examples 46-59

These Examples describe the preparation of ibuprofen/CMCAB solid dispersions and ibuprofen/CMCAB/TPGS solid dispersions. Solid dispersions of containing ibuprofen (IB), carboxymethylcellulose acetate butyrate (CMCAB), and optionally Vitamin E TPGS (TPGS) were prepared by the co-precipitation, flake method of example 1. Specific details of the preparation are listed in Table 4 below.

TABLE 4

| | CMCAB | IB | TPGS | CMCAB | | TPGS | % Crystalline | Wt. % | Wt. % | Wt % |
|---|---|---|---|---|---|---|---|---|---|---|
| Example # | (g)¹ | (g)¹ | (g)¹ | % | IB % | % | IB² | IB³ | CMCAB⁴ | TPGS³ |
| 46 | 4.19 | 1.397 | 0.45 | 69.41 | 23.14 | 7.45 | 6.1 | 22.9 | 72.5 | 4.6 |
| 47 | 6.02 | 0.074 | 0 | 98.79 | 1.21 | 0.00 | 0 | 1.4 | 98.6 | 0 |
| 48 | 5.24 | 0.513 | 0.31 | 86.43 | 8.46 | 5.11 | 2.1 | 6.3 | 90.1 | 3.6 |
| 49 | 5.078 | 0.95 | 0 | 84.24 | 15.76 | 0.00 | 0 | 14.2 | 85.8 | 0 |
| 50 | 4.77 | 0.922 | 0.3 | 79.61 | 15.39 | 5.01 | 0 | 12.1 | 84.5 | 3.4 |
| 51 | 5.504 | 0.08 | 0.6 | 89.00 | 1.29 | 9.70 | 0 | 1.3 | 92.3 | 6.4 |
| 52 | 5.99 | 0.11 | 0 | 98.20 | 1.80 | 0.00 | 0 | 1.8 | 98.2 | 0 |
| 53 | 3.62 | 1.8 | 0.598 | 60.15 | 29.91 | 9.94 | 8.4 | 31.1 | 62.5 | 6.4 |
| 54 | 4.55 | 0.937 | 0.65 | 74.14 | 15.27 | 10.59 | 2.9 | 15.4 | 78.6 | 6 |
| 55 | 4.21 | 1.82 | 0 | 69.82 | 30.18 | 0.00 | 7.9 | 31 | 69 | 0 |
| 56 | 4.2 | 1.8 | 0 | 70.00 | 30.00 | 0.00 | 8.3 | 30.2 | 69.8 | 0 |
| 57 | 5.34 | 0.08 | 0.58 | 89.00 | 1.33 | 9.67 | 0 | 1.9 | 91.6 | 6.5 |
| 58 | 3.6 | 1.83 | 0.6 | 59.70 | 30.35 | 9.95 | 9.6 | 31.8 | 61.3 | 6.9 |
| 59 | 4.51 | 1.43 | 0.151 | 74.04 | 23.48 | 2.48 | 6.7 | 22.9 | 75.2 | 1.9 |

¹Amount added prior to co-precipitation
²Amount in solid dispersions, determined by x-ray
³Amount in solid dispersions, determined by HPLC
⁴Amount is solid dispersions, determined by difference calculations (Wt. % CMCAB = 100 − Wt. % IB − Wt. % TPGS)

### Examples 60-73

These Examples describe the preparation of ibuprofen/CMCAB solid dispersions and ibuprofen/CMCAB/PEG solid dispersions. Solid dispersions of containing ibuprofen (IB), carboxymethylcellulose acetate butyrate (CMCAB), and optionally polyethylene glycol (PEG) were prepared by co-precipitation, the flake method of Example 1. Specific details of the preparation are listed in Table 5 below.

TABLE 5

| | CMCAB | IB | PEG | CMCAB | | PEG | % Crystalline | Wt. % | Wt. % | Wt % |
|---|---|---|---|---|---|---|---|---|---|---|
| Example # | (g)¹ | (g)¹ | (g)¹ | % | IB % | % | IB² | IB³ | CMCAB⁴ | PEG³ |
| 60 | 4.21 | 1.38 | 0.456 | 69.64 | 22.83 | 7.54 | 7.8 | 23.1 | 76.1 | 0.8 |
| 61 | 6.24 | 0.077 | 0 | 98.78 | 1.22 | 0.00 | 0 | 1.5 | 98.5 | 0 |
| 62 | 5.205 | 0.518 | 0.32 | 86.13 | 8.57 | 5.30 | 0 | 8.1 | 89.8 | 2.1 |
| 63 | 5.42 | 0.99 | 0 | 84.56 | 15.44 | 0.00 | 1.7 | 14.5 | 85.5 | 0 |
| 64 | 4.8 | 0.939 | 0.31 | 79.35 | 15.52 | 5.12 | 2.8 | 14.6 | 84.7 | 0.7 |
| 65 | 5.64 | 0.076 | 0.663 | 88.42 | 1.19 | 10.39 | 0 | 1.3 | 93.1 | 5.6 |
| 66 | 5.945 | 0.09 | 0 | 98.51 | 1.49 | 0.00 | 0 | 1.5 | 98.5 | 0 |
| 67 | 3.6 | 1.8 | 0.642 | 59.58 | 29.79 | 10.63 | 11.2 | 35.7 | 62.5 | 1.8 |
| 68 | 4.61 | 0.94 | 0.63 | 74.60 | 15.21 | 10.19 | 2.5 | 15.5 | 83.4 | 1.1 |
| 69 | 4.26 | 1.8 | 0 | 70.30 | 29.70 | 0.00 | 6.8 | 28.1 | 71.9 | 0 |
| 70 | 4.21 | 1.87 | 0 | 69.24 | 30.76 | 0.00 | 8 | 29.6 | 70.4 | 0 |
| 71 | 5.78 | 0.07 | 0.69 | 88.38 | 1.07 | 10.55 | 0 | 1.3 | 94.3 | 4.4 |
| 72 | 3.61 | 1.8 | 0.6 | 60.07 | 29.95 | 9.98 | 6.8 | 30.5 | 68.3 | 1.2 |
| 73 | 4.56 | 1.38 | 0.17 | 74.63 | 22.59 | 2.78 | 4.3 | 21.6 | 77.9 | 0.5 |

¹Amount added prior to co-precipitation
²Amount in solid dispersions, determined by x-ray
³Amount in solid dispersions, determined by HPLC
⁴Amount is solid dispersions, determined by difference calculations (Wt. % CMCAB = 100 − Wt. % IB − Wt. % PEG)

### Examples 74-86

These Examples describe the preparation of phenytoin/polymer solid dispersions and physical blends. Solid dispersions or physical blends of phenytoin (Phe), a polymer carrier (carboxymethylcellulose acetate butyrate (CMCAB), hydroxyproplymethylcellulose acetate succinate (HPMCAS), or cellulose acetate phthalate (C-A-P)) and optionally an additive (Pz) (vitamin E TPGS (TPGS) or sucrose acetate isobutyrate (SAIB)) were prepared by the co-precipitation, flake method of Example (solid dispersions) or by physical mixing (physical blends). Specific details of the preperation are listed in Table 6 below.

TABLE 6

| | | | | | | | | | | | wt % | wt % | wt % |
|---|---|---|---|---|---|---|---|---|---|---|---|---|---|
| | | | | | Drug | Ester | Pz | Drug | Ester | Pz | Crystal | Drug | Pz |
| Example # | Drug | Ester | Pz | Method | (g) | (g) | (g) | (%) | (%) | (%) | (X-ray) | (LC) | (LC) |
| 74 | Phenytoin | CMCAB | None | Flake | 3.63 | 26.49 | 0 | 12.1 | 87.9 | 0.0 | 4.2 | 8.7 | 0 |
| 75 | Phenytoin | CMCAB | TPGS | Flake | 3.61 | 25.13 | 1.58 | 11.9 | 82.9 | 5.2 | 3.1 | 8 | 3.1 |
| 76 | Phenytoin | CMCAB | SAIB | Flake | 3.65 | 25.09 | 1.58 | 12.0 | 82.8 | 5.2 | 3 | 8 | |
| 77 | Phenytoin | CMCAB | None | Phys Blend | 2.4 | 17.7 | 0 | 11.9 | 88.1 | 0.0 | 11.5 | 9.9 | 0 |
| 78 | Phenytoin | None | None | Active | 10 | 0 | 0 | 100.0 | 0.0 | 0.0 | 100 | 94.5 | 0 |
| 79 | Phenytoin | HPMCAS | None | Flake | 3.96 | 26.49 | 0 | 13.0 | 87.0 | 0.0 | 0 | 10 | 0 |
| 80 | Phenytoin | HPMCAS | TPGS | Flake | 3.62 | 24.9 | 1.54 | 12.0 | 82.8 | 5.1 | 0 | 0.1 | 1.6 |
| 81 | Phenytoin | HPMCAS | SAIB | Flake | 3.61 | 24.91 | 1.59 | 12.0 | 82.7 | 5.3 | 1.6 | 9.9 | |
| 82 | Phenytoin | HPMCAS | None | Phys Blend | 2.5 | 17.75 | 0 | 12.3 | 87.7 | 0.0 | 12.5 | 10.7 | 0 |
| 83 | Phenytoin | C-A-P | None | Flake | 3.67 | 24.98 | 0 | 12.8 | 87.2 | 0.0 | 7 | 9.2 | 0 |
| 84 | Phenytoin | C-A-P | TPGS | Flake | 3.66 | 24.98 | 1.95 | 12.0 | 81.7 | 6.4 | | 11.1 | 5.9 |
| 85 | Phenytoin | C-A-P | SAIB | Flake | 3.68 | 24.9 | 1.85 | 12.1 | 81.8 | 6.1 | 11.2 | 9.8 | |
| 86 | Phenytoin | C-A-P | None | Phys Blend | 2.41 | 17.68 | 0 | 12.0 | 88.0 | 0.0 | 12.7 | 11.2 | 0 |

### Examples 87-90

These Examples describe the preparation of phenytoin/polymer solid dispersions by spray drying as described in the Materials and Methods section. Solid dispersions of phenytoin (Phe) and a polymer carrier (carboxymethylcellulose acetate butyrate (CMCAB), hydroxyproplymethylcellulose acetate succinate (HPMCAS), or cellulose acetate phthalate (C-A-P)) were prepared by spray drying. Specific details of the preparation are listed in Table 7, below.

TABLE 7

| | | | | | | | | wt % | wt % |
|---|---|---|---|---|---|---|---|---|---|
| | | | | | Drug | Ester | Acetone | Crystal | Drug |
| Example # | Drug | Ester | Pz | Method | (g) | (g) | (mL) | (X-ray) | (LC) |
| 87 | Phenytoin | CMCAB | None | Spray Dry | 5 | 45.07 | 200 | | |
| 88 | Phenytoin | CMCAB | TPGS | Spray Dry | 5 | 45.18 | 200 | | |
| 89 | Phenytoin | HPMCAS | None | Spray Dry | 5 | 45 | 200 | | |
| 90 | Phenytoin | C-A-P | None | Spray Dry | 5 | 45 | 200 | | |

### Examples 91-99

These Examples describe the preparation of carbamazepine/CMCAB/optional additive solid dispersions and physical blends.

Solid dispersions or physical blends of carbamazepine (Cbz), a polymer carrier (carboxymethylcellulose acetate butyrate (CMCAB)) and optionally an additive (Pz) (vitamin E TPGS (TPGS) or sucrose acetate isobutyrate (SAIB)) were prepared by co-precipitation, co-evaporation, lyophilization, or spray drying (see Examples 1-4 for details) (solid dispersions) or physical mixing (physical blends), as described in Table 8.

TABLE 8

| | | | | | | | | | | | wt % | wt % | wt % |
|---|---|---|---|---|---|---|---|---|---|---|---|---|---|
| | | | | | Drug | Ester | Pz | Drug | Ester | Pz | Crystal | Drug | Pz |
| Example # | Drug | Ester | Pz | Method | (g) | (g) | (g) | (%) | (%) | (%) | (X-ray) | (LC) | (LC) |
| 91 | Carbamazepine | CMCAB | None | Flake | 3.6 | 26.6 | 0 | 11.9 | 88.1 | 0 | 0 | 6.1 | |
| 92 | Carbamazepine | CMCAB | TPGS | Flake | 3.6 | 24.9 | 1.56 | 12.0 | 82.8 | 5.2 | 0 | 6.4 | 3.8 |
| 93 | Carbamazepine | CMCAB | SAIB | Flake | 3.61 | 25.12 | 1.52 | 11.9 | 83.0 | 5.0 | 0 | 6.9 | |
| 94 | Carbamazepine | CMCAB | None | Phys Blend | 2.4 | 17.5 | 0 | 12.1 | 87.9 | 0 | 12.1 | 10 | |
| 95 | Carbamazepine | None | None | Active | 7 | 0 | 0 | 100.0 | 0.0 | 0 | 100 | 90.5 | |
| 96 | Carbamazepine | CMCAB | None | Powder | 7.2 | 52.9 | 0 | 12.0 | 88.0 | 0 | 0 | 4.8 | |
| 97 | Carbamazepine | CMCAB | None | Co-Evp | 7.2 | 52.9 | 0 | 12.0 | 88.0 | 0 | | | |
| 98 | Carbamazepine | CMCAB | None | Lyophilized | 7.2 | 52.9 | 0 | 12.0 | 88.0 | 0 | | | |
| 99 | Carbamazepine | CMCAB | None | Spray | 7.2 | 52.9 | 0 | 12.0 | 88.0 | 0 | | | |

### Examples 100-108

These Examples describe the preparation of nitrofurantoin/polymer/optional additive solid dispersions and physical blends.

Solid dispersions or physical blends of nitrofurantoin (Nit), a polymer carrier (carboxymethylcellulose acetate butyrate (CMCAB)), and optionally an additive (Pz) (vitamin E TPGS (TPGS) or sucrose acetate isobutyrate (SAIB)) were prepared by co-precipitation, co-evaporation, lyophilization, or spray drying (see Examples 1-4 for details (solid dispersions) or physical mixing (physical blends), as described in Table 9.

TABLE 9

| | | | | | | | | | | | wt % | wt % | wt % |
|---|---|---|---|---|---|---|---|---|---|---|---|---|---|
| | | | | | Drug | Ester | Pz | Drug | Ester | Pz | Crystal | Drug | Pz |
| Example # | Drug | Ester | Pz | Method | (g) | (g) | (g) | (%) | (%) | (%) | (X-ray) | (LC) | (LC) |
| 100 | Nitrofurantoin | CMCAB | None | Flake | 3.61 | 26.42 | 0 | 12.0 | 88.0 | 0 | 13.6 | 2.54 | |
| 101 | Nitrofurantoin | CMCAB | TPGS | Flake | 3.61 | 24.92 | 1.58 | 12.0 | 82.8 | 5.2 | 11.7 | 2.73 | 1.34 |
| 102 | Nitrofurantoin | CMCAB | SAIB | Flake | 3.6 | 24.91 | 1.88 | 11.8 | 82.0 | 6.2 | 10.7 | 6.78 | |
| 103 | Nitrofurantoin | CMCAB | None | Phys Blend | 2.4 | 17.72 | 0 | 11.9 | 88.1 | 0 | 11.5 | 9.42 | |
| 104 | Nitrofurantoin | None | None | Active | 10 | 0 | 0 | 100.0 | 0.0 | 0 | 100 | 85.5 | |
| 105 | Nitrofurantoin | CMCAB | None | Powder | 7.4 | 52.8 | 0 | 12.3 | 87.7 | 0 | 9.6 | 7.38 | |
| 106 | Nitrofurantoin | CMCAB | None | Co-Evp | 7.4 | 52.8 | 0 | 12.3 | 87.7 | 0 | | | |
| 107 | Nitrofurantoin | CMCAB | None | Lyophilized | 7.4 | 52.8 | 0 | 12.3 | 87.7 | 0 | | | |
| 108 | Nitrofurantoin | CMCAB | None | Spray | 7.4 | 52.8 | 0 | 12.3 | 87.7 | 0 | | | |

### Examples 109-117

These Examples describe the preparation of glyburide/CMCAB/additive (optional) solid dispersions and physical blends.

Solid dispersions or physical blends of glyburide (Gly), a polymer carrier (carboxymethylcellulose acetate butyrate (CMCAB)), and optionally an additive (Pz) (vitamin E TPGS (TPGS) or sucrose acetate isobutyrate (SAIB)) were prepared by co-precipitation, co-evaporation, lyophilization, or spray drying (see Examples 1-4 for details) (solid dispersions) or physical mixing (physical blends), as described in Table 10. Glyburide was not soluble in acetone and thus DMSO was used to dissolve glyburide. The glyburide/DMSO solution was added to the polymer/additive (optional) solution in acetone prior to formation of the solid dispersion.

TABLE 10

| | | | | | | | | | | | wt % | wt % | wt % | wt % |
|---|---|---|---|---|---|---|---|---|---|---|---|---|---|---|
| | | | | | Drug | Ester | Pz | Drug | Ester | Pz | Crystal | Drug | Pz | DMSO |
| Example # | Drug | Ester | Pz | Method | (g) | (g) | (g) | (%) | (%) | (%) | (X-ray) | (LC) | (LC) | (LC) |
| 109 | Glyburide | CMCAB | None | Flake | 3.61 | 26.48 | 0 | 12.0 | 88.0 | 0 | 0 | 8.88 | | 0.15 |
| 110 | Glyburide | CMCAB | TPGS | Flake | 3.62 | 24.92 | 1.53 | 12.0 | 82.9 | 5.1 | 0 | | | |
| 111 | Glyburide | CMCAB | SAIB | Flake | 3.61 | 24.92 | 1.52 | 12.0 | 82.9 | 5.1 | 0 | | | |
| 112 | Glyburide | CMCAB | None | Phys Blend | 2.4 | 17.5 | | 12.1 | 87.9 | 0 | 12.1 | 10.7 | | 0 |
| 113 | Glyburide | None | None | Active | 5 | 0 | 0 | 100.0 | 0.0 | 0 | 100 | | | |
| 114 | Glyburide | CMCAB | None | Powder | 7.2 | 52.9 | 0 | 12.0 | 88.0 | 0 | | | | |
| 115 | Glyburide | CMCAB | None | Co-Evp | 7.2 | 52.9 | 0 | 12.0 | 88.0 | 0 | | | | |
| 116 | Glyburide | CMCAB | None | Lyophilized | 7.2 | 52.9 | 0 | 12.0 | 88.0 | 0 | | | | |
| 117 | Glyburide | CMCAB | None | Spray | 7.2 | 52.9 | 0 | 12.0 | 88.0 | 0 | | | | |

### Example 118

This Example describes the preparation of a glyburide/CMCAB solid dispersion by spray drying.

A solid dispersion of glyburide (Gly) and a polymer carrier (carboxymethylcellulose acetate butyrate (CMCAB)) was prepared by spray drying as described in Table 11 using the spray drying conditions described in the Materials and Method section. Glyburide was not soluble in acetone and thus DMSO was used to dissolve glyburide. The glyburide/DMSO solution was added to the polymer/additive (optional) solution in acetone prior to formation of the solid dispersion.

TABLE 11

| | Glyburide | Polymer | Polymer | DMSO | Acetone | Wt % | Wt % | % |
|---|---|---|---|---|---|---|---|---|
| Example # | Amount g | Amount g | Description | Amount mL | Amount mL | Gly¹ | DMSO² | Xity³ |
| 118 | 2.5 | 22.5 | CMCAB | 50 | 200 | 7.99 | 10.5* | 0 |

¹Weight percent glyburide in the solid dispersion as determined by HPLC.
²Weight percent DMSO in the solid dispersion as determined by HPLC.
³Percent crystallinity in the solid dispersion as determined by x-ray.

### Examples 119-124

These Examples describe the preparation of glyburide solid dispersions.

Solid dispersions of glyburide (Gly) and a polymer carrier (carboxymethylcellulose acetate butyrate (CMCAB) or hydroxypropylmethylcellulose acetate butyrate (HPMCAS)) were prepared by co-precipitation, flake method (see Example 1 for details) as described in Table 12. Note: Glyburide was not soluble in acetone and thus DMSO was used to dissolve glyburide. The glyburide/DMSO solution was added to the polymer/additive (optional) solution in acetone prior to formation of the solid dispersion.

TABLE 12

| | Glyburide | Polymer | Polymer | DMSO | Acetone | Precipitation | Wt % | Wt % | % |
|---|---|---|---|---|---|---|---|---|---|
| Example # | Amount g | Amount g | Description | Amount mL | Amount mL | Water Amount mL | Gly¹ | DMSO² | Xity³ |
| 119 | 1.08 | 7.92 | CMCAB | 22.5 | 128 | 750 | 9.69 | 1.07 | 100 |
| 120 | 1.08 | 7.92 | CMCAB | 45 | 106 | 750 | 11.1 | 0.34 | 0 |
| 121 | 1.8 | 8 | CMCAB | 22.5 | 128 | 750 | 15.6 | 0.32 | 2.7 |
| 122 | 1.08 | 7.92 | HPMCAS | 22.5 | 128 | 750 | 9.36 | 0.57 | 2.7 |
| 123 | 1.08 | 7.92 | HPMCAS | 45 | 106 | 750 | 9.94 | <0.1 | 0 |
| 124 | 1.8 | 7.2 | HPMCAS | 22.5 | 127.5 | 750 | | | |

¹Weight percent glyburide in the solid dispersion as determined by HPLC.
²Weight percent DMSO in the solid dispersion as determined by HPLC.
³Percent crystallinity in the solid dispersion as determined by x-ray.

### Examples 125-134

These Examples describe the preparation of griseofulvin solid dispersions.

Solid dispersions of griseofulvin (Gris) and a polymer carrier (carboxymethylcellulose acetate butyrate (CMCAB), hydroxypropylmethylcellulose acetate butyrate (HPMCAS), or polyvinylpyrrolidone (PVP)) were prepared by co-precipitation, flake method, or co-evaporation (see Examples 1-4 for details) as described in Table 13.

TABLE 13

| | | | | | | | | | | wt % | wt % |
|---|---|---|---|---|---|---|---|---|---|---|---|
| | | Amount | Solvent for | Amount | | Amount | Solvent for | Amount | Method of | Crystal | Drug |
| Example # | Drug | (g) | Drug | (mL) | Polymer | (g) | Polymer | (mL) | Preparation | (X-ray) | (LC) |
| 125 | Griseofulvin | 1.07 | CH2Cl2 | 15 mL | CMCAB | 9 | Acetone | 50 | Co-evaporation | 0 | 10.2 |
| 126 | Griseofulvin | 1.07 | CH2Cl2 | 15 mL | HPMCAS | 9 | Acetone | 50 | Co-evaporation | | |
| 127 | Griseofulvin | 1.05 | CH2Cl2 | 15 mL | PVP | 9 | Acetonitrile | 50 | Co-evaporation | 0 | 9.87 |
| 128 | Griseofulvin | 1 | CH2Cl2 | 15 mL | CMCAB | 9 | Acetone | 50 | Co-precipitation | 0 | 8.52 |
| 129 | Griseofulvin | 1 | CH2Cl2 | 15 mL | HPMCAS | 9 | Acetone | 50 | Co-precipitation | 0 | 9.21 |
| 130 | Griseofulvin | 1 | CH2Cl2 | 15 mL | CMCAB | 9 | Acetone | 50 | Film co-evaporation | 0 | 9.19 |
| 131 | Griseofulvin | 1 | CH2Cl2 | 15 mL | HPMCAS | 9 | Acetone | 50 | Film co-evaporation | 2.6 | 9.57 |
| 132 | Griseofulvin | 1 | CH2Cl2 | 15 mL | PVP | 9 | Acetonitrile | 50 | Film co-evaporation | 3.4 | 7.88 |
| 133 | Griseofulvin | 1 | CH2Cl2 | 15 mL | CMCAB | 9 | Acetonitrile | 50 | Co-evaporation | 0 | 9.49 |
| 134 | Griseofulvin | 1 | CH2Cl2 | 15 mL | HPMCAS | 9 | Acetonitrile | 50 | Co-evaporation | 0 | 10.4 |

### Examples 135-138

These Examples describe the preparation of griseofulvin/CMCAB/surfactant solid dispersions by co-evaporation.

Solid dispersions of griseofulvin (Gris), a polymer carrier (carboxymethylcellulose acetate butyrate (CMCAB)), and surfactant (Tween 80 or sodium dodecylsulfate (SDS)) were prepared by co-evaporation (see Example 3 for details) as described in Table 14.

TABLE 14

| | | Amount | Solvent for | Amount | | Target | Actual | Solvent for | Amount |
|---|---|---|---|---|---|---|---|---|---|
| Example # | Drug | (g) | Drug | (mL) | Surfactant | Amount (g) | Amount (g) | Surf | (mL) |
| 135 | Griseofulvin | 1 | CH2Cl2 | 15 mL | Tween 80 | 0.1 | 0.5 | Acetone | 10 |
| 136 | Griseofulvin | 1 | CH2Cl2 | 15 mL | Tween 80 | 0.5 | 0.5 | Acetone | 10 |
| 137 | Griseofulvin | 1 | CH2Cl2 | 15 mL | Tween 80 | 0.1 | 0.12 | Acetone | 10 |
| 138 | Griseofulvin | 1 | CH2Cl2 | 15 mL | SDS | 0.5 | 0.5 | CH2Cl2 | 10 |

| | | Target | | | Target | Actual | Actual | | wt % | wt % |
|---|---|---|---|---|---|---|---|---|---|---|
| | | Amount | Solvent for | Dope Conc | Dope | Dope | Polymer | Method of | Crystal | Drug |
| Example # | Polymer | (g) | Polymer | (poly/total) | Amount | Amount | Amount | Prep | (X-ray) | (LC) |
| 135 | CMCAB | 8.9 | Acetone | 0.186690317 | 47.67 | 47.67 | 8.900 | Co-evaporation | | |
| 136 | CMCAB | 8.5 | Acetone | 0.186690317 | 45.53 | 45.55 | 8.504 | Co-evaporation | | |
| 137 | CMCAB | 8.9 | Acetone | 0.186690317 | 47.67 | 47.69 | 8.903 | Co-evaporation | | |
| 138 | CMCAB | 8.5 | Acetone | 0.186690317 | 45.53 | 45.54 | 8.502 | Co-evaporation | | |

### Examples 139-142

These Examples describe the preparation of azithromycin/CMCAB/additive (optional) solid dispersions by co-evaporation.

Solid dispersions of azithromycin (Azi), a polymer carrier (carboxymethylcellulose acetate butyrate (CMCAB)), and an optional additive (surfactant (Tween 80) or vitamin E TPGS (TPGS) were prepared by co-evaporation (see Example 3 for details) as described in Table 15. Immediately upon addition of the azithromycin solution to the CMCAB solution a white precipitate was formed. Upon stirring this precipitate began dissolving, but the solution never completely cleared and remained slightly cloudy. This event was also observed with previous attempts to make CMCAB/azithromycin solid dispersions and many of those attempts were discarded since the precipitation was more pronounced than in this example (i.e. the entire continuer was solidified, but not always white in color, sometimes it would appear as a large gel, this possibly indicated crosslinking or hydrogel formation.

TABLE 15

| | | Amount | Solvent for | Amount | | Target | Actual | Solvent for | Amount |
|---|---|---|---|---|---|---|---|---|---|
| Example # | Drug | (g) | Drug | (mL) | Surfactant | Amount (g) | Amount (g) | Surf | (mL) |
| 139 | Azithromyci [symbol] | 1 | Acetone | 15 mL | | | | Acetone | 10 |
| 140 | Azithromyci [symbol] | 1 | Acetone | 15 mL | Tween 80 | 0.5 | 0.55 | Acetone | 10 |
| 141 | Azithromyci [symbol] | 1 | Acetone | 15 mL | TPGS | 1 | 1 | Acetone | 10 |
| 142 | Azithromyci [symbol] | 1.01 | Acetone | 15 mL | TPGS | 2 | 2.03 | Acetone | 10 |

| | | Target | | | Target | Actual | Actual | | wt % | wt % |
|---|---|---|---|---|---|---|---|---|---|---|
| | | Amount | Solvent for | Dope Conc | Dope | Dope | Polymer | Method of | Crystal | Drug |
| Example # | Polymer | (g) | Polymer | (poly/total) | Amount | Amount | Amount | Prep | (X-ray) | (LC) |
| 139 | CMCAB | 9 | Acetone | 0.186690317 | 48.21 | 48.23 | 9.004 | Co-evaporation | | |
| 140 | CMCAB | 8.5 | Acetone | 0.186690317 | 45.53 | 45.56 | 8.506 | Co-evaporation | | |
| 141 | CMCAB | 8 | Acetone | 0.186690317 | 42.85 | 42.91 | 8.011 | Co-evaporation | | |
| 142 | CMCAB | 7 | Acetone | 0.186690317 | 37.50 | 37.52 | 7.005 | Co-evaporation | | |

Dissolution Studies

### Example 143

These Examples evaluate the samples described in Examples 74-86. SlFsp, pH 6.8 media preparation was described in the Materials and Methods section.

Samples were transferred into vegetable-based Vcaps (Capsugel, size OCS, Lot #630311) using a manual single capsule filler. The weight of each capsule and the amount of sample added are found in Table 16 (Capsule Description).

TABLE 16

| | | Sample Prep | | | | | |
|---|---|---|---|---|---|---|---|
| Sample | Vessel | Notebook | Sample | Capsule | Capsule | Total | Sample |
| # | # | # | Description | Size | Weight g | Weight g | Weight g |
| EX000039-010-1 | 1 | None | Phenytoin | 0 | 0.0971 | 0.1279 | 0.0308 |
| EX000039-010-2 | 2 | None | Phenytoin | 0 | 0.0956 | 0.1239 | 0.0283 |
| EX000039-010-3 | 3 | X-29555-043-1 | Phe/CMCAB SD | 0 | 0.0963 | 0.2121 | 0.1158 |
| EX000039-010-4 | 4 | X-29555-043-1 | Phe/CMCAB SD | 0 | 0.0986 | 0.2062 | 0.1076 |
| EX000039-010-5 | 5 | X-29555-043-1 | Phe/CMCAB SD | 0 | 0.096 | 0.2044 | 0.1084 |
| EX000039-010-6 | 6 | X-29555-043-4 | Phe/CMCAB PB | 0 | 0.0978 | 0.206 | 0.1082 |
| EX000039-010-7 | 7 | X-29555-043-4 | Phe/CMCAB PB | 0 | 0.0974 | 0.2176 | 0.1202 |
| EX000039-010-8 | 8 | X-29555-043-4 | Phe/CMCAB PB | 0 | 0.0967 | 0.2086 | 0.1119 |

Dissolution Protocol. A Varian VK7025 dissolution apparatus and a Varian VK8000 autosampler were used for the dissolution studies using the following parameters: stir rate (50 rpm), sample size (5 mL), sample times (15 min, 30 min, 1 hr, 1 hr 30 min, 2 hr, 3 hr, 4 hr, 5 hr, 6 hr, 24 hr), bath temperature (37.3° C.), vessel temperature (37° C.), pump prime (60 seconds), pump purge (60 seconds), and filter tips (10 μm). The media used was 900 g of SlFsp, pH 6.8 in each vessel.

DMSO (2.0 mL) was added to each test tube in the fraction collector to prevent the drug from recrystallizing once is cooled in the tubes.

All capsules initially floated even though they were inserted into capsule sinkers purchased from Varian. The capsules sank before 15 minutes of mixing.

Samples from Vessels 1 and 3 bounced out of the pill droppers. The sample for vessel 1 was added to the open pill dropper in time to make it into the vessel. The sample for vessel 3 did not get added to the pill dropper before it closed and a separate opening was opened and it was dropped through that opening by hand. Vessel 3 was started approximately 20-30 seconds late.

Several of the capsules did not completely dissolve after 2.5 hours. There was some sample trapped within the following partially dissolved capsules (1, 3, 4, 5, 7, 8).

Since portions of some of the samples were isolated from the dissolution media by being trapped in undissolved VCap capsules these runs were discarded.

### Examples 144-146

These Examples describe the dissolution of carbamazepine and carbamazepine solid dispersions by evaluating the samples described in Examples 91-99. SlFsp, pH 6.8 media preparation was described in the Materials and Methods section.

Samples were transferred into gelatin capsules (Capsugel, size OCS, Lot #624282) using a manual single capsule filler. The weight of each capsule and the amount of sample added are found in Table 17 (Capsule Description).

TABLE 17

| | | Sample Prep | | | | | | |
|---|---|---|---|---|---|---|---|---|
| Sample | Vessel | Notebook | Sample | Capsule | Capsule | Total | Sample | % Drug |
| # | # | # | Description | Size | Weight g | Weight g | Weight g | by LC |
| EX000039-019-1 | 1 | X-29555-043-17 | Cbz/CMCAB PB | 0 | 0.0943 | 0.3092 | 0.2149 | 10 |
| EX000039-019-2 | 2 | X-29555-043-18 | Carbamazepine | 0 | 0.0943 | 0.125 | 0.0307 | 90.5 |
| EX000039-019-3 | 3 | X-29555-043-14 | Cbz/CMCAB SD | 0 | 0.0956 | 0.2654 | 0.1698 | 6.1 |
| EX000039-019-4 | 4 | X-29555-043-17 | Cbz/CMCAB PB | 0 | 0.0968 | 0.4442 | 0.3474 | 10 |
| EX000039-019-5 | 5 | X-29555-043-18 | Carbamazepine | 0 | 0.0955 | 0.1255 | 0.03 | 90.5 |
| EX000039-019-6 | 6 | X-29555-043-17 | Cbz/CMCAB PB | 0 | 0.0948 | 0.3953 | 0.3005 | 10 |
| EX000039-019-7 | 7 | X-29555-043-14 | Cbz/CMCAB SD | 0 | 0.0955 | 0.2834 | 0.1879 | 6.1 |
| EX000039-019-8 | 8 | X-29555-043-14 | Cbz/CMCAB SD | 0 | 0.0948 | 0.2731 | 0.1783 | 6.1 |

TABLE 17A
Total Drug Released (mg)

| | Cbz/CMCAB PB | Carbamazepine | Cbz/CMCAB SD | Cbz/CMCAB PB |
|---|---|---|---|---|
| | Sample # | | | |
| Time | X-29555-043-17 | X-29555-043-18 | X-29555-043-14 | X-29555-043-17 |
| min | EX000039-019-1 | EX000039-019-2 | EX000039-019-3 | EX000039-019-4 |
| 10 | 5.0391 | −1.5539 | −1.5539 | 4.8917 |
| 20 | 13.6043 | 9.4293 | 4.0536 | 18.3350 |
| 30 | 15.4562 | 12.2594 | 6.5810 | 24.4295 |
| 60 | 15.7399 | 16.5605 | 6.7730 | 25.7644 |
| 120 | 15.5921 | 19.0064 | 6.6061 | 25.6305 |
| 180 | 15.4944 | 18.7744 | 6.6931 | 25.7953 |
| 240 | 15.3477 | 18.8275 | 6.5660 | 24.9505 |
| 300 | 15.5894 | 18.7969 | 6.6511 | 24.5158 |
| 360 | 15.4792 | 20.1237 | 6.7339 | 24.7251 |
| 450 | 15.3119 | 18.7429 | 6.6138 | 24.8036 |
| | Carbamazepine | Cbz/CMCAB PB | Cbz/CMCAB SD | Cbz/CMCAB SD |
| | Sample # | | | |
| Time | X-29555-043-18 | X-29555-043-17 | X-29555-043-14 | X-29555-043-14 |
| min | EX000039-019-5 | EX000039-019-6 | EX000039-019-7 | EX000039-019-8 |
| 10 | 5.1967 | 4.5042 | −1.5539 | 0.8162 |
| 20 | 10.5423 | 15.7130 | 1.9622 | 5.0917 |
| 30 | 14.9980 | 20.4260 | 5.0631 | 6.9701 |
| 60 | 18.6664 | 22.5416 | 7.6078 | 7.2212 |
| 120 | 21.6075 | 22.1612 | 8.0073 | 6.9864 |
| 180 | 19.5736 | 22.3220 | 7.4415 | 6.9722 |
| 240 | 19.7250 | 21.8377 | 7.5675 | 6.8960 |
| 300 | 19.7968 | 21.6984 | 7.6193 | 6.8027 |
| 360 | 19.5227 | 21.8670 | 7.4297 | 6.8263 |
| 450 | 19.1984 | 21.8897 | 7.3588 | 6.8926 |

TABLE 17B
% Drug Released

| | Cbz/CMCAB PB | Carbamazepine | Cbz/CMCAB SD | Cbz/CMCAB PB |
|---|---|---|---|---|
| | Sample # | | | |
| Time | X-29555-043-17 | X-29555-043-18 | X-29555-043-14 | X-29555-043-17 |
| min | EX000039-019-1 | EX000039-019-2 | EX000039-019-3 | EX000039-019-4 |
| 10 | 23.4487 | −5.5931 | −15.0027 | 14.0808 |
| 20 | 63.3050 | 33.9384 | 39.1360 | 52.7779 |
| 30 | 71.9229 | 44.1249 | 63.5369 | 70.3209 |
| 60 | 73.2428 | 59.6056 | 65.3899 | 74.1636 |
| 120 | 72.5552 | 68.4090 | 63.7788 | 73.7779 |
| 180 | 72.1007 | 67.5738 | 64.6187 | 74.2525 |
| 240 | 71.4178 | 67.7652 | 63.3917 | 71.8206 |
| 300 | 72.5428 | 67.6548 | 64.2132 | 70.5695 |
| 360 | 72.0299 | 72.4305 | 65.0131 | 71.1718 |
| 450 | 71.2514 | 67.4606 | 63.8535 | 71.3979 |
| | Carbamazepine | Cbz/CMCAB PB | Cbz/CMCAB SD | Cbz/CMCAB SD |
| | Sample # | | | |
| Time | X-29555-043-18 | X-29555-043-17 | X-29555-043-14 | X-29555-043-14 |
| min | EX000039-019-5 | EX000039-019-6 | EX000039-019-7 | EX000039-019-8 |
| 10 | 19.1408 | 14.9891 | −13.5575 | 7.5048 |
| 20 | 38.8298 | 52.2895 | 17.1191 | 46.8147 |
| 30 | 55.2412 | 67.9734 | 44.1736 | 64.0849 |
| 60 | 68.7527 | 75.0136 | 66.3747 | 66.3935 |
| 120 | 79.5856 | 73.7478 | 69.8600 | 64.2355 |
| 180 | 72.0943 | 74.2827 | 64.9239 | 64.1048 |
| 240 | 72.6520 | 72.6713 | 66.0230 | 63.4042 |
| 300 | 72.9162 | 72.2076 | 66.4746 | 62.5460 |
| 360 | 71.9068 | 72.7687 | 64.8209 | 62.7628 |
| 450 | 70.7122 | 72.8443 | 64.2026 | 63.3728 |

TABLE 17C
Average % Released

| | Cbz/CMCAB PB | Carbamazepine | Cbz/CMCAB SD |
|---|---|---|---|
| Time | Sample # | | |
| min | X-29555-043-17 | X-29555-043-18 | X-29555-043-14 |
| 10 | 17.5062 | 6.7739 | −7.0185 |
| 20 | 56.1242 | 36.3841 | 34.3566 |
| 30 | 70.0724 | 49.6830 | 57.2651 |
| 60 | 74.1400 | 64.1792 | 66.0527 |
| 120 | 73.3603 | 73.9973 | 65.9581 |
| 180 | 73.5453 | 69.8341 | 64.5491 |
| 240 | 71.9699 | 70.2086 | 64.2730 |
| 300 | 71.7733 | 70.2855 | 64.4113 |
| 360 | 71.9901 | 72.1686 | 64.1990 |
| 450 | 71.8312 | 69.0864 | 63.8097 |

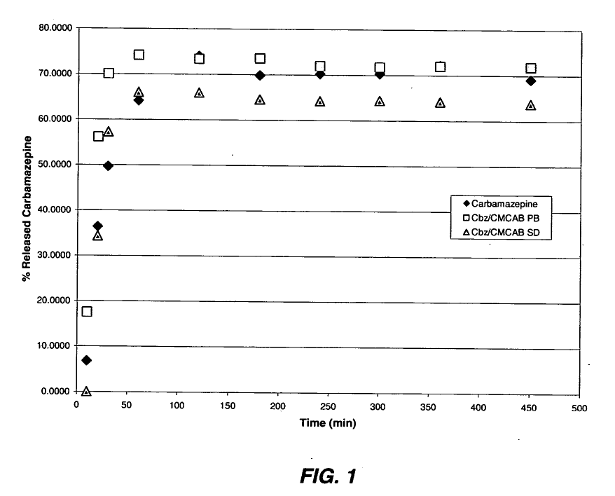

FIG. 1 shows carbamazepine and carbamazepine solid dispersions dissolution profiles.

### Examples 147-149

These Examples describe the dissolution of glyburide and glyburide solid Dispersions by evaluating the samples described in Examples 109-117. SlFsp, pH 6.8 media preperation was described in the Materials and Methods section.

Samples were transferred into gelatin capsules (Capsugel, size OCS, Lot #624282) using a manual single capsule filler. The weight of each capsule and the amount of sample added are found in Table 18 (Capsule Description).

TABLE 18

| | | Sample Prep | | | | | | |
|---|---|---|---|---|---|---|---|---|
| Sample | Vessel | Notebook | Sample | Capsule | Capsule. | Total | Sample | % Drug |
| # | # | # | Description | Size | Weight g | Weight g | Weight g | by LC |
| EX000039-023-1 | 1 | X-29555-043-27 | Glyburide | 0 | 0.0938 | 0.1747 | 0.0809 | 88.9 |
| EX000039-023-2 | 2 | X-29555-043-23 | Gly/CMCAB SD | 0 | 0.0953 | 0.2577 | 0.1624 | 8.49 |
| EX000039-023-3 | 3 | X-29555-043-24 | Gly/CMCAB/TPGS SD | 0 | 0.0951 | 0.3028 | 0.2077 | 8.67 |
| EX000039-023-4 | 4 | X-29555-043-23 | Gly/CMCAB SD | 0 | 0.0955 | 0.2648 | 0.1693 | 8.49 |
| EX000039-023-5 | 5 | X-29555-043-24 | Gly/CMCAB/TPGS SD | 0 | 0.0944 | 0.314 | 0.2196 | 8.67 |
| EX000039-023-6 | 6 | X-29555-043-23 | Gly/CMCAB SD | 0 | 0.0914 | 0.255 | 0.1636 | 8.49 |
| EX000039-023-7 | 7 | X-29555-043-27 | Glyburide | 0 | 0.0934 | 0.177 | 0.0836 | 88.9 |
| EX000039-023-8 | 8 | X-29555-043-24 | Gly/CMCAB/TPGS SD | 0 | 0.0943 | 0.3006 | 0.2063 | 8.67 |

TABLE 18A
Total Drug Released (mg)

| | Glyburide | Gly/CMCAB SD | Gly/CMCAB/TPGS SD | Gly/CMCAB SD |
|---|---|---|---|---|
| | Sample # | | | |
| Time | X-29555-043-27 | X-29555-043-23 | X-29555-043-24 | X-29555-043-23 |
| min | EX000039-023-1 | EX000039-023-2 | EX000039-023-3 | EX000039-023-4 |
| 10 | −1.5219 | −1.5219 | 2.2056 | −0.6987 |
| 20 | −0.8018 | 4.4084 | 8.8578 | 4.5814 |
| 30 | −0.6491 | 6.5687 | 9.9189 | 6.5636 |
| 60 | −0.2329 | 7.6080 | 10.8926 | 7.7988 |
| 120 | 0.0521 | 7.9981 | | 8.2094 |
| 180 | 0.1504 | 8.0920 | 11.1159 | 8.3416 |
| 240 | 0.1934 | 8.0570 | 11.0773 | 8.3189 |
| 300 | 0.2583 | 8.0754 | 11.1055 | 8.3242 |
| 360 | 0.3246 | 8.0909 | 11.0082 | 8.2953 |
| 450 | | 8.0015 | 11.1217 | 8.3772 |

| | Gly/CMCAB/ | | | Gly/CMCAB/ |
|---|---|---|---|---|
| | TPGS SD | Gly/CMCAB SD | Glyburide | TPGS SD |
| | Sample # | | | |
| Time | X-29555-043-24 | X-29555-043-23 | X-29555-043-27 | X-29555-043-24 |
| min | EX000039-023-5 | EX000039-023-6 | EX000039-023-7 | EX000039-023-8 |
| 10 | 2.5357 | −0.5469 | −1.2990 | 3.2672 |
| 20 | 8.6164 | 5.0459 | −0.7920 | 9.3445 |
| 30 | 9.8214 | 6.5916 | −0.6233 | 10.1650 |
| 60 | 11.2513 | 7.6496 | −0.2559 | 10.7942 |
| 120 | 11.5971 | 8.0137 | −0.0751 | 11.0344 |
| 180 | 11.6435 | 8.0299 | 0.0469 | 10.9419 |
| 240 | 11.6367 | 8.0399 | 0.1252 | 10.9735 |
| 300 | 11.5940 | 8.1190 | 0.1982 | 10.9481 |
| 360 | 11.6018 | 8.0899 | 0.2373 | 10.9629 |
| 450 | 11.6217 | 8.0627 | 0.3205 | 10.8221 |

TABLE 18B
Normalized Drug Released (mg)

| | Glyburide | Gly/CMCAB SD | Gly/CMCAB/TPGS SD | Gly/CMCAB SD |
|---|---|---|---|---|
| | Sample # | | | |
| Time | X-29555-043-27 | X-29555-043-23 | X-29555-043-24 | X-29555-043-23 |
| min | EX000039-023-1 | EX000039-023-2 | EX000039-023-3 | EX000039-023-4 |
| 10 | 0.0000 | 0.0000 | 3.7275 | 0.8232 |
| 20 | 0.7201 | 5.9303 | 10.3797 | 6.1033 |
| 30 | 0.8728 | 8.0906 | 11.4409 | 8.0855 |
| 60 | 1.2890 | 9.1299 | 12.4145 | 9.3207 |
| 120 | 1.5740 | 9.5200 | | 9.7313 |
| 180 | 1.6724 | 9.6139 | 12.6378 | 9.8636 |
| 240 | 1.7153 | 9.5789 | 12.5992 | 9.8409 |
| 300 | 1.7802 | 9.5973 | 12.6274 | 9.8461 |
| 360 | 1.8465 | 9.6128 | 12.5301 | 9.8172 |
| 450 | | 9.5234 | 12.6436 | 9.8991 |

| | Gly/CMCAB/ | | | Gly/CMCAB/ |
|---|---|---|---|---|
| | TPGS SD | Gly/CMCAB SD | Glyburide | TPGS SD |
| | Sample # | | | |
| Time | X-29555-043-24 | X-29555-043-23 | X-29555-043-27 | X-29555-043-24 |
| min | EX000039-023-5 | EX000039-023-6 | EX000039-023-7 | EX000039-023-8 |
| 10 | 4.0576 | 0.9750 | 0.2229 | 4.7891 |
| 20 | 10.1383 | 6.5679 | 0.7299 | 10.8665 |
| 30 | 11.3433 | 8.1135 | 0.8986 | 11.6869 |
| 60 | 12.7732 | 9.1715 | 1.2660 | 12.3161 |
| 120 | 13.1190 | 9.5356 | 1.4468 | 12.5563 |
| 180 | 13.1654 | 9.5518 | 1.5688 | 12.4638 |
| 240 | 13.1586 | 9.5618 | 1.6471 | 12.4954 |
| 300 | 13.1160 | 9.6409 | 1.7202 | 12.4701 |
| 360 | 13.1237 | 9.6119 | 1.7592 | 12.4848 |
| 450 | 13.1436 | 9.5846 | 1.8424 | 12.3440 |

TABLE 18C

| | Glyburide | Gly/CMCAB SD | Gly/CMCAB/TPGS SD | Gly/CMCAB SD |
|---|---|---|---|---|
| | Sample # | | | |
| Time | X-29555-043-27 | X-29555-043-23 | X-29555-043-24 | X-29555-043-23 |
| min | EX000039-023-1 | EX000039-023-2 | EX000039-023-3 | EX000039-023-4 |
| 10 | 0.0000 | 0.0000 | 20.6995 | 5.7275 |
| 20 | 1.0013 | 43.0114 | 57.6409 | 42.4619 |
| 30 | 1.2136 | 58.6797 | 63.5335 | 56.2526 |
| 60 | 1.7923 | 66.2174 | 68.9403 | 64.8463 |
| 120 | 2.1886 | 69.0469 | | 67.7027 |
| 180 | 2.3253 | 69.7281 | 70.1806 | 68.6229 |
| 240 | 2.3850 | 69.4741 | 69.9659 | 68.4650 |
| 300 | 2.4752 | 69.6075 | 70.1227 | 68.5014 |
| 360 | 2.5674 | 69.7199 | 69.5825 | 68.3005 |
| 450 | | 69.0716 | 70.2126 | 68.8701 |

| | Gly/CMCAB/ | | | Gly/CMCAB/ |
|---|---|---|---|---|
| | TPGS SD | Gly/CMCAB SD | Glyburide | TPGS SD |
| | Sample # | | | |
| Time | X-29555-043-24 | X-29555-043-23 | X-29555-043-27 | X-29555-043-24 |
| min | EX000039-023-5 | EX000039-023-6 | EX000039-023-7 | EX000039-023-8 |
| 10 | 21.3119 | 7.0196 | 0.3099 | 26.7756 |
| 20 | 53.2495 | 47.2861 | 1.0149 | 60.7533 |
| 30 | 59.5783 | 58.4143 | 1.2495 | 65.3404 |
| 60 | 67.0887 | 66.0314 | 1.7603 | 68.8582 |
| 120 | 68.9047 | 68.6524 | 2.0117 | 70.2010 |
| 180 | 69.1485 | 68.7690 | 2.1813 | 69.6837 |
| 240 | 69.1129 | 68.8413 | 2.2902 | 69.8606 |
| 300 | 68.8888 | 69.4108 | 2.3918 | 69.7188 |
| 360 | 68.9294 | 69.2017 | 2.4461 | 69.8014 |
| 450 | 69.0342 | 69.0055 | 2.5617 | 69.0141 |

TABLE 18D
Average % Released

| | Glyburide | Gly/CMCAB SD | Gly/CMCAB/TPGS SD |
|---|---|---|---|
| Time | Sample # | | |
| min | X-29555-043-27 | X-29555-043-23 | X-29555-043-24 |
| 10 | 0.1549 | 4.2490 | 22.9290 |
| 20 | 1.0081 | 44.2531 | 57.2146 |
| 30 | 1.2315 | 57.7822 | 62.8174 |
| 60 | 1.7763 | 65.6984 | 68.2957 |
| 120 | 2.1001 | 68.4673 | 69.5529 |
| 180 | 2.2533 | 69.0400 | 69.6710 |
| 240 | 2.3376 | 68.9268 | 69.6465 |
| 300 | 2.4335 | 69.1732 | 69.5768 |
| 360 | 2.5067 | 69.0740 | 69.4378 |
| 450 | 2.5617 | 68.9824 | 69.4203 |

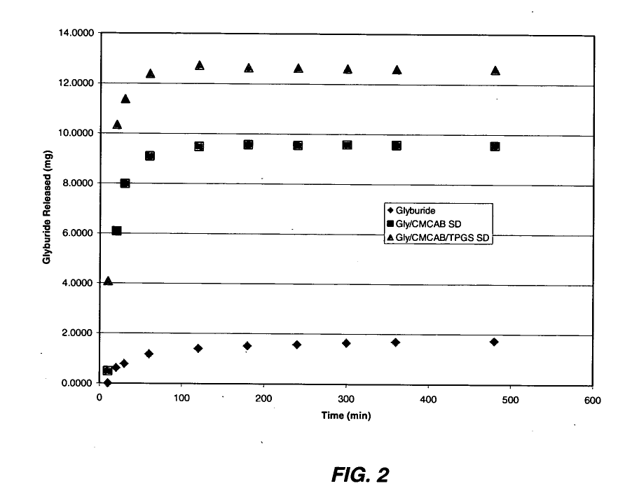

FIG. 2 shows glyburide and glyburide solid dispersions dissolution profiles.

### Examples 150-155

These Examples describe the dissolution of glyburide and glyburide solid dispersions by evaluating the samples described in Examples 119-124. SIFsp, pH 6.8 media preperation was described in the Materials and Methods section.

Samples were transferred into gelatin capsules (Capsugel, size OCS, Lot # 624282) using a manual single capsule filler. The weight of each capsule and the amount of sample added are found in Tables 19 and 20 (Capsule Description).

TABLE 19

| | Vessel | Sample Prep | | Capsule |
|---|---|---|---|---|
| Sample # | # | Notebook # | Sample Description | Size |
| EX000039-044-1 | 1 | Lt024K0701 | Glyburide | 0 |
| EX000039-044-2 | 2 | EX000039-036-1 | Gly/CMCAB SD | 0 |
| EX000039-044-3 | 3 | Lt024K0701 | Glyburide | 0 |
| EX000039-044-4 | 4 | EX000039-036-1 | Gly/CMCAB SD | 0 |
| EX000039-044-5 | 5 | | | |
| EX000039-044-6 | 6 | EX000039-036-1 | Gly/CMCAB SD | 0 |
| EX000039-044-7 | 7 | Lt024K0701 | Glyburide | 0 |
| EX000039-044-8 | 8 | EX000039-036-1 | Gly/CMCAB SD | 0 |
| EX000039-045-1 | 1 | EX000039-036-2 | CMCAB/Glyburide SD | 0 |
| EX000039-045-2 | 2 | EX000039-036-3 | CMCAB/Glyburide SD | 0 |
| EX000039-045-3 | 3 | EX000039-036-2 | CMCAB/Glyburide SD | 0 |
| EX000039-045-4 | 4 | EX000039-036-3 | CMCAB/Glyburide SD | 0 |
| EX000039-045-5 | 5 | | | |
| EX000039-045-6 | 6 | EX000039-036-3 | CMCAB/Glyburide SD | 0 |
| EX000039-045-7 | 7 | EX000039-036-2 | CMCAB/Glyburide SD | 0 |
| EX000039-045-8 | 8 | EX000039-036-2 | CMCAB/Glyburide SD | 0 |
| EX000039-046-1 | 1 | EX000039-036-4 | HPMCAS/Glyburide SD | 0 |
| EX000039-046-2 | 2 | EX000039-036-5 | HPMCAS/Glyburide SD | 0 |
| EX000039-046-3 | 3 | EX000039-036-4 | HPMCAS/Glyburide SD | 0 |
| EX000039-046-4 | 4 | EX000039-036-5 | HPMCAS/Glyburide SD | 0 |
| EX000039-046-5 | 5 | | | |
| EX000039-046-6 | 6 | EX000039-036-5 | HPMCAS/Glyburide SD | 0 |
| EX000039-046-7 | 7 | EX000039-036-4 | HPMCAS/Glyburide SD | 0 |
| EX000039-046-8 | 8 | EX000039-036-4 | HPMCAS/Glyburide SD | 0 |

TABLE 20

| | Capsule | Total | Sample | % Drug | Drug in | DMSO | |
|---|---|---|---|---|---|---|---|
| Sample # | Weight g | Weight g | Weight g | by LC | Capsule mg | Wt % | % Xity |
| EX000039-044-1 | 0.0961 | 0.1895 | 0.0934 | 96.9 | 90.5046 | 0 | 0 |
| EX000039-044-2 | 0.0955 | 0.3475 | 0.252 | 9.69 | 24.4188 | 1.07 | 0 |
| EX000039-044-3 | 0.0917 | 0.174 | 0.0823 | 96.9 | 79.7487 | 0 | 0 |
| EX000039-044-4 | 0.0922 | 0.3306 | 0.2384 | 9.69 | 23.10096 | 1.07 | 0 |
| EX000039-044-5 | | | | | | | |
| EX000039-044-6 | 0.0931 | 0.3414 | 0.2483 | 9.69 | 24.06027 | 1.07 | 0 |
| EX000039-044-7 | 0.0951 | 0.1867 | 0.0916 | 96.9 | 88.7604 | 0 | 0 |
| EX000039-044-8 | 0.0942 | 0.3407 | 0.2465 | 9.69 | 23.88585 | 1.07 | 0 |
| EX000039-045-1 | 0.0941 | 0.1991 | 0.105 | 11.10 | 11.6550 | 0.34 | 2.7 |
| EX000039-045-2 | 0.092 | 0.2333 | 0.1413 | 15.60 | 22.0428 | 0.32 | 2.7 |
| EX000039-045-3 | 0.0945 | 0.2308 | 0.1363 | 11.10 | 15.1293 | 0.34 | 2.7 |
| EX000039-045-4 | 0.0955 | 0.213 | 0.1175 | 15.60 | 18.3300 | 0.32 | 2.7 |
| EX000039-045-5 | | | | | | | |
| EX000039-045-6 | 0.0944 | 0.2317 | 0.1373 | 15.60 | 21.4188 | 0.32 | 2.7 |
| EX000039-045-7 | 0.0952 | 0.2261 | 0.1309 | 11.10 | 14.5299 | 0.34 | 2.7 |
| EX000039-045-8 | 0.0952 | 0.185 | 0.0898 | 11.10 | 9.9678 | 0.34 | 2.7 |
| EX000039-046-1 | 0.0946 | 0.3414 | 0.2468 | 9.36 | 0.0231 | 0.57 | 0 |
| EX000039-046-2 | 0.0934 | 0.205 | 0.1116 | 9.94 | 0.0111 | <0.2 | 0 |
| EX000039-046-3 | 0.0937 | 0.3079 | 0.2142 | 9.36 | 0.0200 | 0.57 | 0 |
| EX000039-046-4 | 0.0936 | 0.2048 | 0.1112 | 9.94 | 0.0111 | <0.2 | 0 |
| EX000039-046-5 | | | | | | | |
| EX000039-046-6 | 0.0953 | 0.205 | 0.1097 | 9.94 | 0.0109 | <0.2 | 0 |
| EX000039-046-7 | 0.0947 | 0.2744 | 0.1797 | 9.36 | 0.0168 | 0.57 | 0 |
| EX000039-046-8 | 0.0913 | 0.3322 | 0.2409 | 9.36 | 0.0225 | 0.57 | 0 |

TABLE 20A
Total Drug Released (mg)

| | Glyburide | Gly/CMCAB SD | Glyburide | Gly/CMCAB SD |
|---|---|---|---|---|
| | Sample # | | | |
| Time | Drug | EX000039-036-1 | Drug | EX000039-036-1 |
| min | EX000039-044-1 | EX000039-044-2 | EX000039-044-3 | EX000039-044-4 |
| 15 | 0.0321 | 2.7022 | 0.0321 | 0.8907 |
| 30 | 0.0321 | 6.3951 | 0.2322 | 3.3345 |
| 45 | 0.3834 | 10.0485 | 0.3424 | 6.1538 |
| 60 | 0.3950 | 13.0845 | 0.4562 | 8.7641 |
| 90 | 0.6124 | 17.5758 | 0.6166 | 13.2085 |
| 120 | 0.7134 | 20.1525 | 0.7222 | 16.6634 |
| 150 | 0.8270 | 22.2370 | 0.8369 | 19.5955 |
| 180 | 0.8919 | 23.7455 | 0.9402 | |
| 210 | 0.9428 | 24.1593 | 0.9956 | 22.6921 |
| 240 | 1.0358 | 24.2168 | 1.0726 | 23.1819 |
| | | Gly/CMCAB SD | Glyburide | Gly/CMCAB SD |
| | Sample # | | | |
| Time | | EX000039-036-1 | Drug | EX000039-036-1 |
| min | EX000039-044-5 | EX000039-044-6 | EX000039-044-7 | EX000039-044-8 |
| 15 | | 3.0134 | 0.0321 | 8.3353 |
| 30 | | 7.3343 | 0.3730 | 13.1771 |
| 45 | | 11.0312 | 0.4071 | 16.7701 |
| 60 | | 14.0083 | 0.5752 | 19.6024 |
| 90 | | 18.0834 | 0.7677 | 23.4151 |
| 120 | | 20.4571 | 0.8356 | 24.3092 |
| 150 | | 21.9013 | 0.9711 | 24.6604 |
| 180 | | 23.2828 | 0.9572 | 24.7122 |
| 210 | | 23.9530 | 1.4143 | 24.3535 |
| 240 | | 23.9340 | 1.2243 | 24.1000 |

TABLE 20B
% Drug Released

| | Glyburide | Gly/CMCAB SD | Glyburide | Gly/CMCAB SD |
|---|---|---|---|---|
| | Sample # | | | |
| Time | Drug | EX000039-036-1 | Drug | EX000039-036-1 |
| min | EX000039-044-1 | EX000039-044-2 | EX000039-044-3 | EX000039-044-4 |
| 10 | 0.0355 | 11.0661 | 0.0403 | 3.8558 |
| 20 | 0.0355 | 26.1892 | 0.2912 | 14.4343 |
| 30 | 0.4237 | 41.1508 | 0.4293 | 26.6389 |
| 60 | 0.4365 | 53.5839 | 0.5720 | 37.9384 |
| 120 | 0.6767 | 71.9764 | 0.7732 | 57.1775 |
| 180 | 0.7882 | 82.5286 | 0.9056 | 72.1328 |
| 240 | 0.9138 | 91.0651 | 1.0495 | 84.8255 |
| 300 | 0.9855 | 97.2428 | 1.1790 | |
| 360 | 1.0417 | 98.9374 | 1.2484 | 98.2302 |
| 450 | 1.1444 | 99.1726 | 1.3449 | 100.3505 |
| | | Gly/CMCAB SD | Glyburide | Gly/CMCAB SD |
| | Sample # | | | |
| Time | | EX000039-036-1 | Drug | EX000039-036-1 |
| min | EX000039-044-5 | EX000039-044-6 | EX000039-044-7 | EX000039-044-8 |
| 10 | | 12.5244 | 0.0362 | 34.8964 |
| 20 | | 30.4830 | 0.4202 | 55.1671 |
| 30 | | 45.8482 | 0.4587 | 70.2093 |
| 60 | | 58.2219 | 0.6480 | 82.0669 |
| 120 | | 75.1586 | 0.8649 | 98.0293 |
| 180 | | 85.0243 | 0.9414 | 101.7723 |
| 240 | | 91.0269 | 1.0941 | 103.2428 |
| 300 | | 96.7688 | 1.0784 | 103.4596 |
| 360 | | 99.5542 | 1.5934 | 101.9579 |
| 450 | | 99.4753 | 1.3793 | 100.8966 |

TABLE 20C
Total Drug Released (mg)

| | Gly/CMCAB SD | Gly/CMCAB SD | Gly/CMCAB SD | Gly/CMCAB SD |
|---|---|---|---|---|
| | Sample # | | | |
| Time | EX000039-036-2 | EX000039-036-3 | EX000039-036-2 | EX000039-036-3 |
| min | EX000039-045-1 | EX000039-045-2 | EX000039-045-3 | EX000039-045-4 |
| 15 | 2.0196 | 4.9485 | 3.1565 | 4.5326 |
| 30 | 4.0273 | 8.3712 | 5.0765 | 7.5472 |
| 45 | 4.4796 | 9.1719 | 5.4931 | 8.4260 |
| 60 | 4.8707 | 9.6898 | 5.8042 | 8.9389 |
| 90 | 5.2491 | 10.1842 | 6.0701 | 9.4626 |
| 120 | 5.6053 | 10.6236 | 6.3825 | 9.7103 |
| 150 | 5.6803 | 11.1267 | 6.6169 | 10.1284 |
| 180 | 5.8419 | 11.1766 | 6.7375 | 10.2111 |
| 210 | 5.9831 | 11.2241 | 6.8028 | 10.2809 |
| 240 | 5.9575 | 11.3209 | 6.7890 | 10.6076 |
| | | Gly/CMCAB SD | Gly/CMCAB SD | Gly/CMCAB SD |
| | Sample # | | | |
| Time | | EX000039-036-3 | EX000039-036-2 | EX000039-036-2 |
| min | EX000039-045-5 | EX000039-045-6 | EX000039-045-7 | EX000039-045-8 |
| 15 | | 4.7500 | 2.8471 | 2.3841 |
| 30 | | 8.0297 | 4.7618 | 3.9051 |
| 45 | | 8.8857 | 5.4658 | 4.4487 |
| 60 | | 9.4029 | 5.7254 | 4.7834 |
| 90 | | 9.9966 | 6.1215 | 5.0237 |
| 120 | | 10.3182 | 6.5590 | 5.1859 |
| 150 | | 10.5440 | 6.6215 | 5.3959 |
| 180 | | 10.8004 | 6.7478 | 5.5955 |
| 210 | | 10.7875 | 6.7590 | 5.5819 |
| 240 | | 11.2664 | 6.8465 | 5.7242 |

TABLE 20D
% Drug Released

| | Gly/CMCAB SD | Gly/CMCAB SD | Gly/CMCAB SD | Gly/CMCAB SD |
|---|---|---|---|---|
| | Sample # | | | |
| Time | EX000039-036-2 | EX000039-036-3 | EX000039-036-2 | EX000039-036-3 |
| min | EX000039-045-1 | EX000039-045-2 | EX000039-045-3 | EX000039-045-4 |
| 10 | 17.3284 | 22.4494 | 20.8636 | 24.7279 |
| 20 | 34.5543 | 37.9770 | 33.5541 | 41.1742 |
| 30 | 38.4351 | 41.6097 | 36.3080 | 45.9685 |
| 60 | 41.7911 | 43.9590 | 38.3640 | 48.7663 |
| 120 | 45.0369 | 46.2021 | 40.1214 | 51.6238 |
| 180 | 48.0935 | 48.1952 | 42.1864 | 52.9749 |
| 240 | 48.7374 | 50.4775 | 43.7357 | 55.2556 |
| 300 | 50.1239 | 50.7043 | 44.5326 | 55.7072 |
| 360 | 51.3350 | 50.9197 | 44.9645 | 56.0879 |
| 450 | 51.1150 | 51.3589 | 44.8732 | 57.8703 |
| | | Gly/CMCAB SD | Gly/CMCAB SD | Gly/CMCAB SD |
| | Sample # | | | |
| Time | | EX000039-036-3 | EX000039-036-2 | EX000039-036-2 |
| min | EX000039-045-5 | EX000039-045-6 | EX000039-045-7 | EX000039-045-8 |
| 10 | | 22.1766 | 19.5946 | 23.9182 |
| 20 | | 37.4889 | 32.7722 | 39.1773 |
| 30 | | 41.4855 | 37.6176 | 44.6310 |
| 60 | | 43.9004 | 39.4043 | 47.9888 |
| 120 | | 46.6723 | 42.1301 | 50.3990 |
| 180 | | 48.1734 | 45.1413 | 52.0267 |
| 240 | | 49.2280 | 45.5714 | 54.1329 |
| 300 | | 50.4250 | 46.4409 | 56.1362 |
| 360 | | 50.3645 | 46.5182 | 55.9994 |
| 450 | | 52.6004 | 47.1200 | 57.4266 |

TABLE 20E
Total Drug Released (mg)

| | HPMCAS/Gly SD | HPMCAS/Gly SD | HPMCAS/Gly SD | HPMCAS/Gly SD |
|---|---|---|---|---|
| | Sample # | | | |
| Time | EX000039-036-4 | EX000039-036-5 | EX000039-036-4 | EX000039-036-5 |
| min | EX000039-046-1 | EX000039-046-2 | EX000039-046-3 | EX000039-046-4 |
| 15 | 1.1011 | 7.4108 | 1.3791 | 6.5816 |
| 30 | 4.4528 | 9.4128 | 4.3607 | 9.1836 |
| 45 | 7.2066 | 9.9065 | 6.9892 | 9.7294 |
| 60 | 9.6549 | 10.1545 | 9.0370 | 9.9747 |
| 90 | 13.7393 | 10.5025 | 11.9870 | 10.1525 |
| 120 | 16.9871 | 10.4761 | 14.3403 | 10.2741 |
| 150 | 19.7324 | 10.4162 | 15.9981 | 10.4222 |
| 180 | 21.7776 | 10.5196 | 17.7797 | 10.4192 |
| 210 | 22.9188 | 10.5084 | 18.9679 | 10.6124 |
| 240 | 23.8843 | 10.6079 | 20.0208 | 10.5921 |

| | | |
|---|---|---|
| HPMCAS/Gly SD | HPMCAS/Gly SD | HPMCAS/Gly SD |

| | Sample # | | | |
|---|---|---|---|---|
| Time | | EX000039-036-5 | EX000039-036-4 | EX000039-036-4 |
| min | EX000039-046-5 | EX000039-046-6 | EX000039-046-7 | EX000039-046-8 |
| 15 | | 7.5154 | 2.3387 | 3.2577 |
| 30 | | 9.2272 | 5.3869 | 6.9338 |
| 45 | | 9.8914 | 7.8544 | 10.1164 |
| 60 | | 10.1524 | 9.8341 | 13.0096 |
| 90 | | 10.2225 | 12.7487 | 17.2764 |
| 120 | | 10.3754 | 14.9230 | 20.3482 |
| 150 | | 10.4490 | 16.3234 | 22.4369 |
| 180 | | 10.3862 | 17.4505 | 23.3829 |
| 210 | | 10.4925 | 17.6811 | 23.9734 |
| 240 | | 10.4778 | 17.9006 | 24.0551 |

TABLE 20F
% Drug Released

| | HPMCAS/Gly SD | HPMCAS/Gly SD | HPMCAS/Gly SD | HPMCAS/Gly SD |
|---|---|---|---|---|
| | Sample # | | | |
| Time | EX000039-036-4 | EX000039-036-5 | EX000039-036-4 | EX000039-036-5 |
| min | EX000039-046-1 | EX000039-046-2 | EX000039-046-3 | EX000039-046-4 |
| 10 | 4.7665 | 66.8058 | 6.8788 | 59.5442 |
| 20 | 19.2758 | 84.8530 | 21.7503 | 83.0850 |
| 30 | 31.1968 | 89.3039 | 34.8606 | 88.0229 |
| 60 | 41.7954 | 91.5397 | 45.0745 | 90.2422 |
| 120 | 59.4764 | 94.6766 | 59.7884 | 91.8501 |
| 180 | 73.5356 | 94.4382 | 71.5258 | 92.9511 |
| 240 | 85.4197 | 93.8981 | 79.7944 | 94.2909 |
| 300 | 94.2735 | 94.8311 | 88.6805 | 94.2637 |
| 360 | 99.2134 | 94.7300 | 94.6074 | 96.0113 |
| 450 | 103.3931 | 95.6271 | 99.8590 | 95.8273 |

| | | |
|---|---|---|
| HPMCAS/Gly SD | HPMCAS/Gly SD | HPMCAS/Gly SD |

| | Sample # | | | |
|---|---|---|---|---|
| Time | | EX000039-036-5 | EX000039-036-4 | EX000039-036-4 |
| min | EX000039-046-5 | EX000039-046-6 | EX000039-046-7 | EX000039-046-8 |
| 10 | | 68.9218 | 13.9042 | 14.4477 |
| 20 | | 84.6211 | 32.0272 | 30.7510 |
| 30 | | 90.7117 | 46.6967 | 44.8654 |
| 60 | | 93.1052 | 58.4672 | 57.6969 |
| 120 | | 93.7488 | 75.7954 | 76.6197 |
| 180 | | 95.1511 | 88.7220 | 90.2431 |
| 240 | | 95.8255 | 97.0480 | 99.5061 |
| 300 | | 95.2497 | 103.7493 | 103.7018 |
| 360 | | 96.2242 | 105.1201 | 106.3206 |
| 450 | | 96.0901 | 106.4250 | 106.6827 |

TABLE 20G
Average % Released

| | Glyburide | Gly/CMCAB SD | Gly/CMCAB SD | Gly/CMCAB SD | HPMCAS/Gly SD | HPMCAS/Gly SD |
|---|---|---|---|---|---|---|
| Time | Sample # | | | | | |
| min | Drug | EX000039-036-1 | EX000039-036-2 | EX000039-036-3 | EX000039-036-4 | EX000039-036-5 |
| 10 | 0.0373 | 15.5857 | 20.4262 | 23.3180 | 9.9993 | 65.0906 |
| 20 | 0.2490 | 31.5684 | 35.0145 | 38.9544 | 25.9511 | 84.1864 |
| 30 | 0.4372 | 45.9618 | 39.2480 | 43.4237 | 39.4049 | 89.3462 |
| 60 | 0.5522 | 57.9528 | 41.8870 | 46.1537 | 50.7585 | 91.6290 |
| 120 | 0.7716 | 75.5854 | 44.4219 | 48.7243 | 67.9200 | 93.4252 |
| 180 | 0.8784 | 85.3645 | 46.8620 | 50.3425 | 81.0066 | 94.1801 |
| 240 | 1.0191 | 92.5401 | 48.0443 | 52.2735 | 90.4421 | 94.6715 |
| 300 | 1.0810 | 99.1571 | 49.3084 | 53.2432 | 97.6013 | 94.7815 |
| 360 | 1.2945 | 99.6699 | 49.7043 | 53.3429 | 101.3154 | 95.6551 |
| 450 | 1.2896 | 99.9738 | 50.1337 | 54.8141 | 104.0899 | 95.8482 |

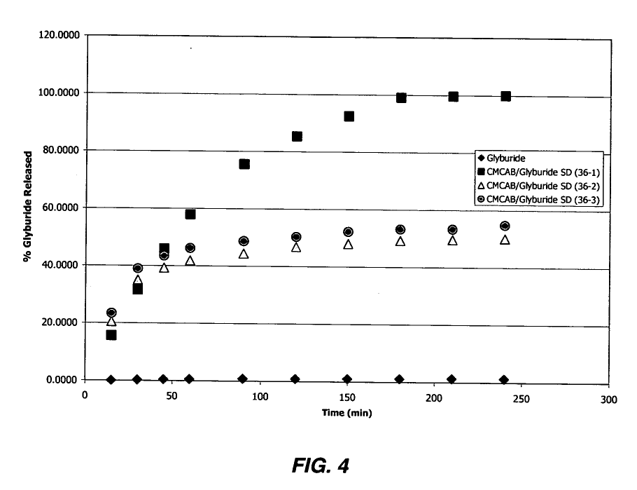

FIG. 4 is the dissolution profile of three different CMCAB/glyburide solid dispersions (see Tables 19 and 20 for the description of each sample). Both samples with lower dissolution rates (EX000039-036-2 and -036-3) were solid dispersions with approximately 2.7% crystallinity. It is possible that the increase in crystallinity caused the decrease in release rate and total amount of glyburide released into the media. Other process factors could have also played a role in the reduced release rate.

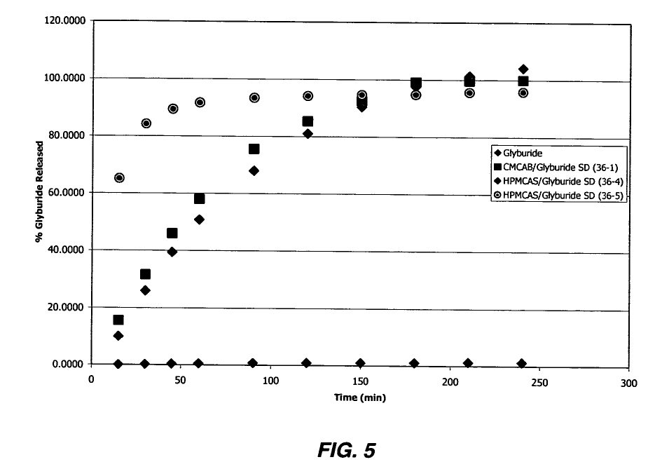

FIG. 5 contains the dissolution profiles of two HPMCAS/glyburide solid dispersions compared with the best performing CMCAB/glyburide solid dispersion and glyburide (see Tables 19 and 20 for the description of each sample). It is obvious from these results that the release rate of glyburide can be modified within a group of solid dispersions using the same polymeric carrier and that CMCAB can perform equally as well as HPMCAS in certain systems.

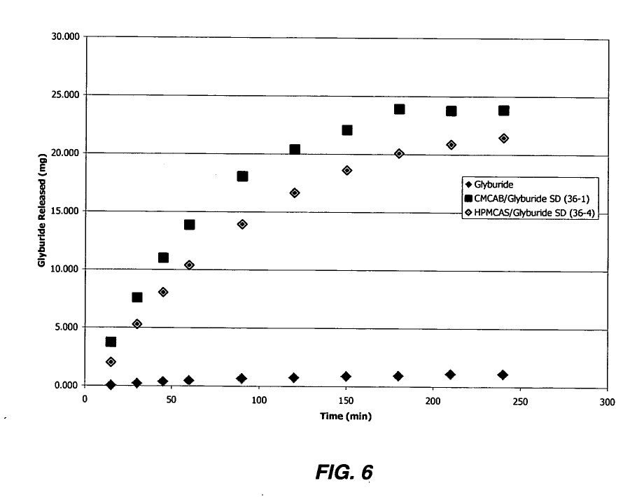

The mass of glyburide released from a CMCAB/glyburide solid dispersion and a HPMCAS/glyburide solid dispersion is presented in FIG. 6. When viewing the data in this manner it is obvious that CMCAB/glyburide solid dispersion (EX000039-036-1) performed better than the HPMCAS/glyburide solid dispersion (EX000039-036-4). The HPMCAS/glyburide solid dispersion (EX000039-036-5) outperforms both of these samples as can be seen in FIG. 5. In the format of FIG. 6, the 036-5 sample had a lower drug loading than the 036-1 or 036-4 samples.

### Examples 156-161

These Examples describe the dissolution of griseofulvin and griseofulvin solid dispersions by evaluating the samples described in Examples 125-134. SlFsp, pH 6.8 media preparation was described in the Materials and Methods section.

Samples were transferred into gelatin capsules (Capsugel, size 0CS, Lot # 624282) using a manual single capsule filler. The weight of each capsule and the amount of sample added are found in Table 21 and 22 (Capsule Description).

TABLE 21

| | Vessel | Sample Prep | Sample | Sample | Capsule |
|---|---|---|---|---|---|
| Sample # | # | Notebook # | Description | Prep | Size |
| EX000039-047-1 | 1 | 083K1219 | Griseofulvin | Drug | 0 |
| EX000039-047-2 | 2 | EX000039-040-1 | CMCAB/Gris SD | Co-evp | 0 |
| EX000039-047-3 | 3 | 083K1219 | Griseofulvin | Drug | 0 |
| EX000039-047-4 | 4 | EX000039-040-1 | CMCAB/Gris SD | Co-evp | 0 |
| EX000039-047-5 | 5 | 083K1219 | Griseofulvin | Drug | 0 |
| EX000039-047-6 | 6 | EX000039-040-1 | CMCAB/Gris SD | Co-evp | 0 |
| EX000039-047-7 | 7 | 083K1219 | Griseofulvin | Drug | 0 |
| EX000039-047-8 | 8 | EX000039-040-1 | CMCAB/Gris SD | Co-evp | 0 |
| EX000039-048-1 | 1 | EX000039-040-3 | PVP/Gris SD | Co-evp | 0 |
| EX000039-048-2 | 2 | EX000039-040-4 | CMCAB/Gris SD | Ppt | 0 |
| EX000039-048-3 | 3 | EX000039-040-3 | PVP/Gris SD | Co-evp | 0 |
| EX000039-048-4 | 4 | EX000039-040-4 | CMCAB/Gris SD | Ppt | 0 |
| EX000039-048-5 | 5 | EX000039-040-3 | PVP/Gris SD | Co-evp | 0 |
| EX000039-048-6 | 6 | EX000039-040-4 | CMCAB/Gris SD | Ppt | 0 |
| EX000039-048-7 | 7 | EX000039-040-3 | PVP/Gris SD | Co-evp | 0 |
| EX000039-048-8 | 8 | EX000039-040-4 | CMCAB/Gris SD | Ppt | 0 |
| EX000039-049-1 | 1 | EX000039-040-5 | HPMCAS/Gris SD | Ppt | 0 |
| EX000039-049-2 | 2 | EX000039-040-6 | CMCAB/Gris SD | Film Co-evp | 0 |
| EX000039-049-3 | 3 | EX000039-040-5 | HPMCAS/Gris SD | Ppt | 0 |
| EX000039-049-4 | 4 | EX000039-040-6 | CMCAB/Gris SD | Film Co-evp | 0 |
| EX000039-049-5 | 5 | EX000039-040-5 | HPMCAS/Gris SD | Ppt | 0 |
| EX000039-049-6 | 6 | EX000039-040-6 | CMCAB/Gris SD | Film Co-evp | 0 |
| EX000039-049-7 | 7 | EX000039-040-5 | HPMCAS/Gris SD | Ppt | 0 |
| EX000039-049-8 | 8 | EX000039-040-6 | CMCAB/Gris SD | Film Co-evp | 0 |

TABLE 22

| | Capsule | Total | Sample | % Drug | Drug in | |
|---|---|---|---|---|---|---|
| Sample # | Weight g | Weight g | Weight g | by LC | Capsule mg | % Xity |
| EX000039-047-1 | 0.0949 | 0.2914 | 0.1965 | 96.4 | 189.4 | 100 |
| EX000039-047-2 | 0.0959 | 0.4337 | 0.3378 | 10.2 | 34.5 | 0 |
| EX000039-047-3 | 0.0944 | 0.2863 | 0.1919 | 96.4 | 185.0 | 100 |
| EX000039-047-4 | 0.0963 | 0.4305 | 0.3342 | 10.2 | 34.1 | 0 |
| EX000039-047-5 | 0.0941 | 0.286 | 0.1919 | 96.4 | 185.0 | 100 |
| EX000039-047-6 | 0.0938 | 0.4163 | 0.3225 | 10.2 | 32.9 | 0 |
| EX000039-047-7 | 0.0955 | 0.3227 | 0.2272 | 96.4 | 219.0 | 100 |
| EX000039-047-8 | 0.0951 | 0.4133 | 0.3182 | 10.2 | 32.5 | 0 |
| EX000039-048-1 | 0.0952 | 0.4526 | 0.3574 | 9.87 | 35.3 | 0 |
| EX000039-048-2 | 0.0934 | 0.4127 | 0.3193 | 8.52 | 27.2 | 0 |
| EX000039-048-3 | 0.0949 | 0.4596 | 0.3647 | 9.87 | 36.0 | 0 |
| EX000039-048-4 | 0.095 | 0.4194 | 0.3244 | 8.52 | 27.6 | 0 |
| EX000039-048-5 | 0.0951 | 0.4285 | 0.3334 | 9.87 | 32.9 | 0 |
| EX000039-048-6 | 0.0954 | 0.3682 | 0.2728 | 8.52 | 23.2 | 0 |
| EX000039-048-7 | 0.094 | 0.3751 | 0.2811 | 9.87 | 27.7 | 0 |
| EX000039-048-8 | 0.0946 | 0.4226 | 0.328 | 8.52 | 27.9 | 0 |
| EX000039-049-1 | 0.0952 | 0.5188 | 0.4236 | 9.21 | 39.0 | 0 |
| EX000039-049-2 | 0.0949 | 0.5029 | 0.408 | 9.19 | 37.5 | 0 |
| EX000039-049-3 | 0.0952 | 0.4959 | 0.4007 | 9.21 | 36.9 | 0 |
| EX000039-049-4 | 0.0947 | 0.4978 | 0.4031 | 9.19 | 37.0 | 0 |
| EX000039-049-5 | 0.0962 | 0.4942 | 0.398 | 9.21 | 36.7 | 0 |
| EX000039-049-6 | 0.095 | 0.4948 | 0.3998 | 9.19 | 36.7 | 0 |
| EX000039-049-7 | 0.0953 | 0.5333 | 0.438 | 9.21 | 40.3 | 0 |
| EX000039-049-8 | 0.0935 | 0.5239 | 0.4304 | 9.19 | 39.6 | 0 |

TABLE 22A
Total Drug Released (mg)

| | Griseofulvin | Gris/CMCAB SD | Griseofulvin | Gris/CMCAB SD |
|---|---|---|---|---|
| | Sample # | | | |
| Time | Griseofulvin | EX000039-040-1 | Griseofulvin | EX000039-040-1 |
| min | EX000039-047-1 | EX000039-047-2 | EX000039-047-3 | EX000039-047-4 |
| 15 | 12.9809 | 1.5609 | 12.9579 | 2.0980 |
| 30 | 13.1347 | 4.4468 | 13.2291 | 4.2917 |
| 45 | 12.9970 | 6.2791 | 13.2561 | 6.0074 |
| 60 | 13.2187 | 8.1057 | 13.2865 | 7.6437 |
| 90 | 12.8793 | 10.2209 | 13.0152 | 9.3797 |
| 120 | 13.8093 | 11.9735 | 13.2937 | 11.1139 |
| 150 | 13.0036 | 12.8185 | 12.9639 | 11.9355 |
| 180 | 12.9786 | 13.6019 | 12.8966 | 12.7607 |
| 210 | 13.1671 | 14.3534 | 12.9476 | 13.5760 |
| 240 | 13.0461 | 15.2645 | 13.2489 | 14.4654 |
| | Griseofulvin | Gris/CMCAB SD | Griseofulvin | Gris/CMCAB SD |
| | Sample # | | | |
| Time | Griseofulvin | EX000039-040-1 | Griseofulvin | EX000039-040-1 |
| min | EX000039-047-5 | EX000039-047-6 | EX000039-047-7 | EX000039-047-8 |
| 15 | 13.1651 | 1.9474 | 12.7375 | 1.8564 |
| 30 | 13.1014 | 3.3056 | 13.0986 | 4.2574 |
| 45 | 13.0726 | 4.6003 | 13.1471 | 6.2941 |
| 60 | 13.3296 | 5.7322 | 12.9703 | 7.8380 |
| 90 | 13.1633 | 7.3397 | 14.2638 | 10.6032 |
| 120 | 13.3211 | 8.7390 | 13.2009 | 11.6810 |
| 150 | 13.1075 | 9.2870 | 13.3283 | 12.7176 |
| 180 | 12.9315 | 9.7942 | 13.0464 | 13.6898 |
| 210 | 13.0903 | 10.5153 | 13.0333 | 14.9911 |
| 240 | 13.0897 | 11.4187 | 12.8881 | 15.7045 |

TABLE 22B
% Drug Released

| | Griseofulvin | Gris/CMCAB SD | Griseofulvin | Gris/CMCAB SD |
|---|---|---|---|---|
| | Sample # | | | |
| Time | Griseofulvin | EX000039-040-1 | Griseofulvin | EX000039-040-1 |
| min | EX000039-047-1 | EX000039-047-2 | EX000039-047-3 | EX000039-047-4 |
| 15 | 36.6133 | 5.7043 | 35.8167 | 7.5465 |
| 30 | 37.0470 | 16.2505 | 36.5664 | 15.4371 |
| 45 | 36.6587 | 22.9467 | 36.6412 | 21.6086 |
| 60 | 37.2839 | 29.6218 | 36.7251 | 27.4942 |
| 90 | 36.3266 | 37.3517 | 35.9752 | 33.7387 |
| 120 | 38.9498 | 43.7564 | 36.7450 | 39.9765 |
| 150 | 36.6774 | 46.8445 | 35.8335 | 42.9319 |
| 180 | 36.6068 | 49.7073 | 35.6474 | 45.8999 |
| 210 | 37.1385 | 52.4534 | 35.7882 | 48.8328 |
| 240 | 36.7973 | 55.7831 | 36.6212 | 52.0319 |
| | Griseofulvin | Gris/CMCAB SD | Griseofulvin | Gris/CMCAB SD |
| | Sample # | | | |
| Time | Griseofulvin | EX000039-040-1 | Griseofulvin | EX000039-040-1 |
| min | EX000039-047-5 | EX000039-047-6 | EX000039-047-7 | EX000039-047-8 |
| 15 | 39.8059 | 8.3298 | 45.3135 | 6.6573 |
| 30 | 39.6132 | 14.1394 | 46.9736 | 15.2675 |
| 45 | 39.5262 | 19.6769 | 47.1475 | 22.5714 |
| 60 | 40.3033 | 24.5187 | 46.5134 | 28.1082 |
| 90 | 39.8005 | 31.3946 | 51.1520 | 38.0244 |
| 120 | 40.2775 | 37.3797 | 47.3403 | 41.8896 |
| 150 | 39.6316 | 39.7236 | 47.7972 | 45.6070 |
| 180 | 39.0996 | 41.8930 | 46.7861 | 49.0935 |
| 210 | 39.5796 | 44.9776 | 46.7393 | 53.7603 |
| 240 | 39.5780 | 48.8419 | 46.2187 | 56.3185 |

TABLE 22C
Total Drug Released (mg)

| | Gris/PVP SD Co- | Gris/CMCAB SD | Gris/PVP SD Co- | Gris/CMCAB SD |
|---|---|---|---|---|
| | evap | Co-Ppt | evap | Co-Ppt |
| | Sample # | | | |
| Time | EX000039-040-3 | EX000039-040-4 | EX000039-040-3 | EX000039-040-4 |
| min | EX000039-048-1 | EX000039-048-2 | EX000039-048-3 | EX000039-048-4 |
| 15 | 9.9996 | 1.4755 | 9.6753 | 2.1650 |
| 30 | 17.4239 | 3.7589 | 16.8334 | 3.3415 |
| 45 | 17.4059 | 5.3141 | 17.2502 | 4.9486 |
| 60 | 17.3946 | 6.5307 | 17.2214 | 6.5650 |
| 90 | 17.2406 | 8.5005 | 16.8140 | 8.9330 |
| 120 | 17.2740 | 10.7522 | 17.8023 | 11.3360 |
| 150 | 16.9607 | 11.5109 | 16.6208 | 13.0561 |
| 180 | 16.7757 | 12.7906 | 16.2912 | 13.6839 |
| 210 | 16.7851 | 14.0867 | 16.8391 | 14.7480 |
| 240 | 16.4131 | 15.0130 | 16.4544 | 15.9036 |
| | Gris/PVP SD Co- | Gris/CMCAB SD | Gris/PVP SD Co- | Gris/CMCAB SD |
| | evap | Co-Ppt | evap | Co-Ppt |
| | Sample # | | | |
| Time | EX000039-040-3 | EX000039-040-4 | EX000039-040-3 | EX000039-040-4 |
| min | EX000039-048-5 | EX000039-048-6 | EX000039-048-7 | EX000039-048-8 |
| 15 | 9.7745 | 2.0648 | 8.6415 | 1.7164 |
| 30 | 16.3982 | 3.8999 | 15.0999 | 4.0500 |
| 45 | 17.2819 | 5.7631 | 15.8053 | 5.9299 |
| 60 | 17.1839 | 7.2092 | 15.7985 | 7.5201 |
| 90 | 17.0428 | 9.3872 | 15.9514 | 10.0257 |
| 120 | 17.0698 | 10.8863 | 16.1015 | 11.3604 |
| 150 | 16.7958 | 12.3264 | 16.0268 | 12.7934 |
| 180 | 16.6404 | 13.5326 | 15.6296 | 14.2034 |
| 210 | 16.8336 | 14.0898 | 15.5183 | 15.5169 |
| 240 | 16.4408 | 15.0170 | 15.5550 | 16.5624 |

TABLE 22D
% Drug Released

| | Gris/PVP SD Co- | Gris/CMCAB SD | Gris/PVP SD Co- | Gris/CMCAB SD |
|---|---|---|---|---|
| | evap | Co-Ppt | evap | Co-Ppt |
| | Sample # | | | |
| Time | EX000039-040-3 | EX000039-040-4 | EX000039-040-3 | EX000039-040-4 |
| min | EX000039-048-1 | EX000039-048-2 | EX000039-048-3 | EX000039-048-4 |
| 15 | 28.2045 | 5.3922 | 26.7435 | 7.7876 |
| 30 | 49.1450 | 13.7368 | 46.5291 | 12.0191 |
| 45 | 49.0943 | 19.4201 | 47.6812 | 17.8000 |
| 60 | 49.0623 | 23.8659 | 47.6015 | 23.6143 |
| 90 | 48.6281 | 31.0644 | 46.4755 | 32.1318 |
| 120 | 48.7221 | 39.2934 | 49.2073 | 40.7754 |
| 150 | 47.8385 | 42.0658 | 45.9413 | 46.9626 |
| 180 | 47.3168 | 46.7425 | 45.0303 | 49.2209 |
| 210 | 47.3431 | 51.4789 | 46.5449 | 53.0485 |
| 240 | 46.2941 | 54.8640 | 45.4814 | 57.2051 |
| | Gris/PVP SD Co- | Gris/CMCAB SD | Gris/PVP SD Co- | Gris/CMCAB SD |
| | evap | Co-Ppt | evap | Co-Ppt |
| | Sample # | | | |
| Time | EX000039-040-3 | EX000039-040-4 | EX000039-040-3 | EX000039-040-4 |
| min | EX000039-048-5 | EX000039-048-6 | EX000039-048-7 | EX000039-048-8 |
| 15 | 29.5542 | 8.8319 | 30.9895 | 6.1061 |
| 30 | 49.5814 | 16.6811 | 54.1505 | 14.4080 |
| 45 | 52.2535 | 24.6508 | 56.6799 | 21.0955 |
| 60 | 51.9572 | 30.8364 | 56.6556 | 26.7529 |
| 90 | 51.5304 | 40.1525 | 57.2038 | 35.6663 |
| 120 | 51.6120 | 46.5647 | 57.7423 | 40.4148 |
| 150 | 50.7835 | 52.7241 | 57.4745 | 45.5126 |
| 180 | 50.3138 | 57.8836 | 56.0501 | 50.5288 |
| 210 | 50.8979 | 60.2670 | 55.6508 | 55.2015 |
| 240 | 49.7104 | 64.2330 | 55.7826 | 58.9206 |

TABLE 22E
Total Drug Released (mg)

| | Gris/HPMCAS SD | Grts/CMCAB SD | Gris/HPMCAS SD | Gris/CMCAB SD |
|---|---|---|---|---|
| | Co-Ppt | Co-Evap (film) | Co-Ppt | Co-Evap (film) |
| | Sample # | | | |
| Time | EX000039-040-5 | EX000039-040-6 | EX000039-040-5 | EX000039-040-6 |
| min | EX000039-049-1 | EX000039-049-2 | EX000039-049-3 | EX000039-049-4 |
| 15 | 4.9721 | 1.2834 | 14.2108 | 0.9289 |
| 30 | 5.7511 | 2.8784 | 15.6603 | 2.4705 |
| 45 | 5.9501 | 4.2490 | 16.5603 | 3.5984 |
| 60 | 6.1222 | 5.5960 | 16.9882 | 4.7561 |
| 90 | 6.5943 | 7.3894 | 16.7500 | 6.3535 |
| 120 | 6.7926 | 8.7812 | 17.1461 | 7.8829 |
| 150 | 7.1275 | 10.1074 | 17.4313 | 9.0446 |
| 180 | 7.4120 | 10.8039 | 17.8693 | 9.8346 |
| 210 | 7.5936 | 11.8673 | 17.6789 | 10.8286 |
| 240 | 7.8820 | 12.4033 | 17.8073 | 11.2821 |
| | Gris/HPMCAS SD | Gris/CMCAB SD | Gris/HPMCAS SD | Gris/CMCAB SD |
| | Co-Ppt | Co-Evap (film) | Co-Ppt | Co-Evap (film) |
| | Sample # | | | |
| Time | EX000039-040-5 | EX000039-040-6 | EX000039-040-5 | EX000039-040-6 |
| min | EX000039-049-5 | EX000039-049-6 | EX000039-049-7 | EX000039-049-8 |
| 15 | | 0.8137 | 5.4854 | 1.0343 |
| 30 | | 2.5240 | 7.3840 | 2.9252 |
| 45 | | 3.7953 | 7.9958 | 4.3061 |
| 60 | | 4.7919 | 8.2168 | 5.1880 |
| 90 | | 6.3212 | 8.6812 | 6.7430 |
| 120 | | 7.3077 | 9.2581 | 7.8737 |
| 150 | | 8.1396 | 9.5011 | 8.9107 |
| 180 | | 8.8441 | 9.6101 | 9.4227 |
| 210 | | 9.2020 | 9.8162 | 10.3873 |
| 240 | | 9.8209 | 10.0703 | 10.8388 |

TABLE 22F
% Drug Released

| | Gris/HPMCAS SD | Gris/CMCAB SD | Gris/HPMCAS SD | Gris/CMCAB SD |
|---|---|---|---|---|
| | Co-Ppt | Co-Evap (film) | Co-Ppt | Co-Evap (film) |
| | Sample # | | | |
| Time | EX000039-040-5 | EX000039-040-6 | EX000039-040-5 | EX000039-040-6 |
| min | EX000039-049-1 | EX000039-049-2 | EX000039-049-3 | EX000039-049-4 |
| 15 | 14.0240 | 4.6900 | 39.2799 | 3.3414 |
| 30 | 16.2212 | 10.5188 | 43.2866 | 8.8862 |
| 45 | 16.7827 | 15.5278 | 45.7741 | 12.9432 |
| 60 | 17.2681 | 20.4502 | 46.9570 | 17.1077 |
| 90 | 18.5996 | 27.0040 | 46.2985 | 22.8535 |
| 120 | 19.1588 | 32.0905 | 47.3935 | 28.3547 |
| 150 | 20.1035 | 36.9367 | 48.1817 | 32.5333 |
| 180 | 20.9060 | 39.4821 | 49.3923 | 35.3748 |
| 210 | 21.4182 | 43.3681 | 48.8662 | 38.9505 |
| 240 | 22.2316 | 45.3271 | 49.2211 | 40.5816 |
| | Gris/HPMCAS SD | Gris/CMCAB SD | Gris/HPMCAS SD | Gris/CMCAB SD |
| | Co-Ppt | Co-Evap (film) | Co-Ppt | Co-Evap (film) |
| | Sample # | | | |
| Time | EX000039-040-5 | EX000039-040-6 | EX000039-040-5 | EX000039-040-6 |
| min | EX000039-049-5 | EX000039-049-6 | EX000039-049-7 | EX000039-049-8 |
| 15 | | 3.4804 | 19.6716 | 3.7091 |
| 30 | | 10.7959 | 26.4801 | 10.4903 |
| 45 | | 16.2339 | 28.6740 | 15.4424 |
| 60 | | 20.4968 | 29.4665 | 18.6047 |
| 90 | | 27.0380 | 31.1322 | 24.1815 |
| 120 | | 31.2576 | 33.2008 | 28.2361 |
| 150 | | 34.8161 | 34.0724 | 31.9550 |
| 180 | | 37.8294 | 34.4632 | 33.7910 |
| 210 | | 39.3600 | 35.2021 | 37.2502 |
| 240 | | 42.0076 | 36.1135 | 38.8696 |

TABLE 22G
Average % Released

| | | | Gris/PVP SD Co- | Gris/CMCAB SD | Gris/HPMCAS SD | Gris/CMCAB SD |
|---|---|---|---|---|---|---|
| | Griseofulvin | Gris/CMCAB SD | evap | Co-Ppt | Co-Ppt | Co-Evap (film) |
| | Sample # | | | | | |
| Time | Griseofulvin | EX000039-040-1 | EX000039-040-3 | EX000039-040-4 | EX000039-040-5 | EX000039-040-6 |
| min | MS-39-47 | MS-39-47 | MS-39-48 | MS-39-48 | MS-39-44 | MS-39-44 |
| 15 | 39.3874 | 7.0595 | 28.8729 | 7.0295 | 24.3252 | 3.8052 |
| 30 | 40.0500 | 15.2736 | 49.8515 | 14.2112 | 28.6626 | 10.1728 |
| 45 | 39.9934 | 21.7009 | 51.4272 | 20.7416 | 30.4103 | 15.0368 |
| 60 | 40.2064 | 27.4357 | 51.3191 | 26.2674 | 31.2305 | 19.1649 |
| 90 | 40.8136 | 35.1274 | 50.9595 | 34.7537 | 32.0101 | 25.2693 |
| 120 | 40.8282 | 40.7505 | 51.8209 | 41.7621 | 33.2511 | 29.9847 |
| 150 | 39.9849 | 43.7767 | 50.5094 | 46.8163 | 34.1192 | 34.0603 |
| 180 | 39.5350 | 46.6484 | 49.6777 | 51.0940 | 34.9205 | 36.6194 |
| 210 | 39.8114 | 50.0060 | 50.1092 | 54.9990 | 35.1622 | 39.7322 |
| 240 | 39.8038 | 53.2438 | 49.3171 | 58.8057 | 35.8554 | 41.6965 |

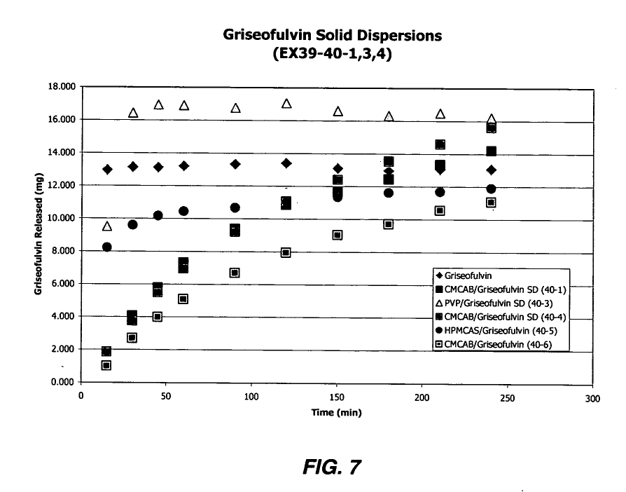

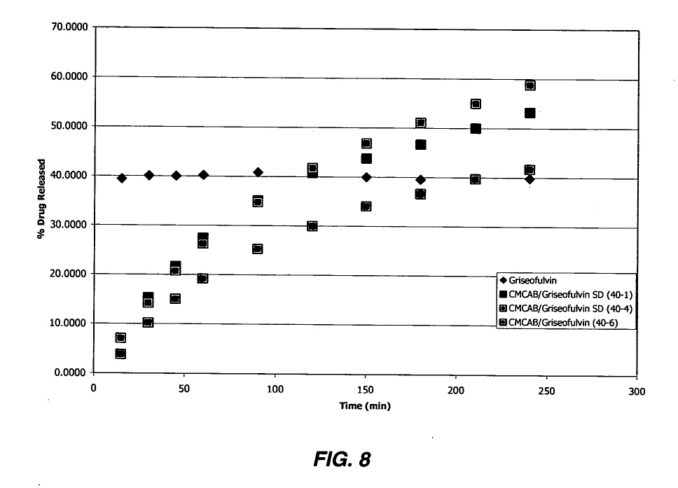

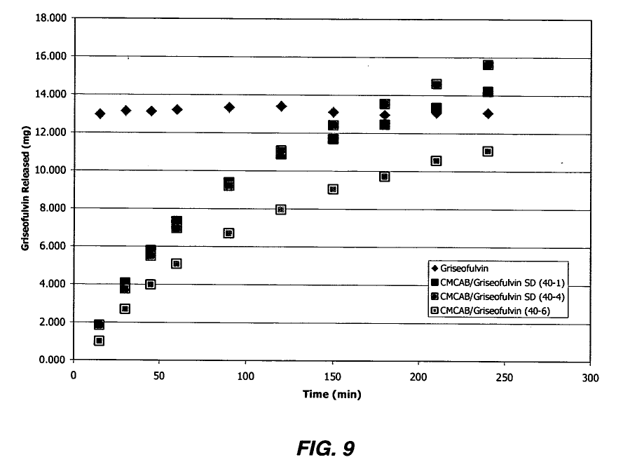

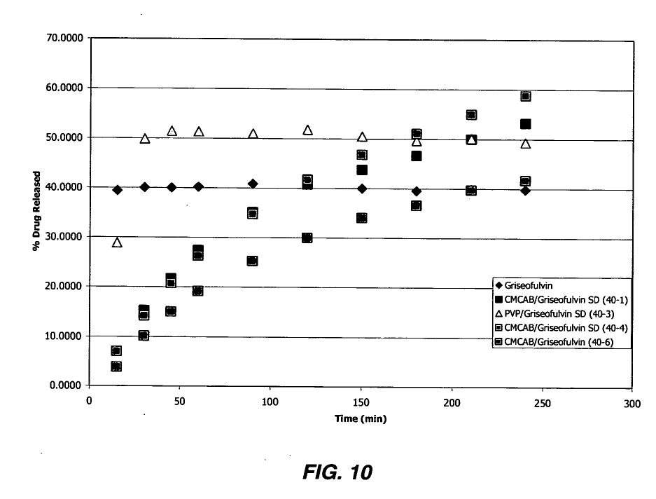

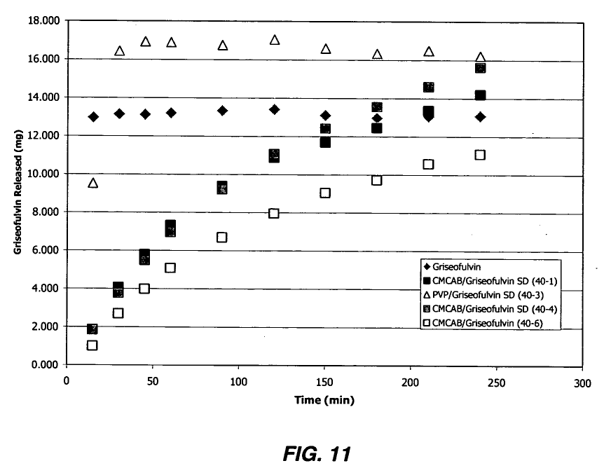

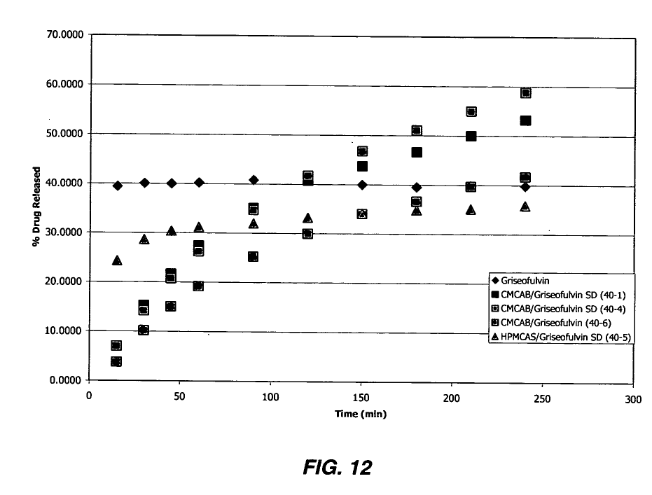

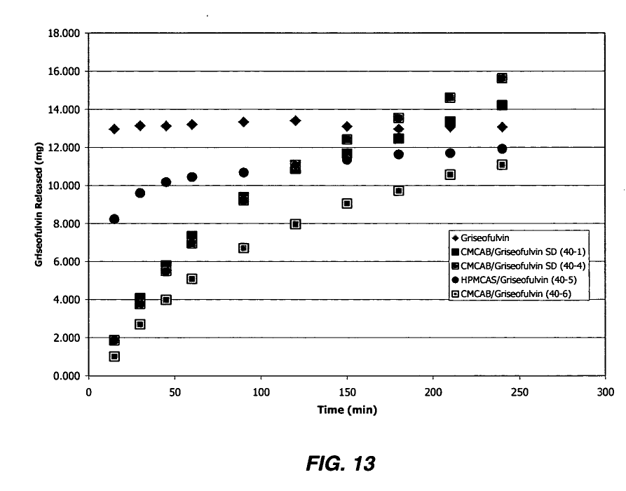

Griseofulvin was released into SIFsp, pH 6.8 media in a controlled and sustained manner that differed from the immediate release observed with the unmodified drug substance (FIGS. 7-9). CMCAB/griseofulvin solid dispersions on have a different release profile than that of CMCAB/glyburide solid dispersions. Griseofulvin release from a CMCAB solid dispersion showed a more controlled release than the rapid release of griseofulvin from PVP solid dispersions, HPMCAS solid dispersion, or unmodified griseofulvin (FIGS. 7, 10-13). However, the total amount of griseofulvin released was not improved by the formation of the solid dispersions.

### Examples 162-164

These Examples describe the impact of surfactant additives on the dissolution profiles of griseofulvin/CMCAB solid dispersions. Preparations of the griseofulvin/CMCAB, griseofulvin/CMCAB/Tween 80, and griseofulvin/CMCAB/SDS solid dispersions evaluated in this example are described in Examples 135-138. SIFsp, pH 6.8 media preparation was described in the Materials and Methods section.

Samples were transferred into gelatin capsules (Capsugel, size 00CS, Lot # 637785) using a manual single capsule filler. The weight of each capsule and the amount of sample added are found in Table 23 (Capsule Description).

TABLE 23

| Example | Dissolution | Total Drug | Sample Prep | Sample | % Drug |
|---|---|---|---|---|---|
| # | Run # | in Capsule | # | Description | calc'd |
| 162-A | EX000039-056-1 | 50.916 | EX000039-053-1 | Gris/CMCAB/Tween | 9.62 |
| 163-A | EX000039-056-2 | 51.181 | EX000039-053-2 | Gris/CMCAB/Tween | 10.00 |
| 162-B | EX000039-056-3 | 74.600 | EX000039-053-1 | Gris/CMCAB/Tween | 9.62 |
| 163-B | EX000039-056-4 | 61.277 | EX000039-053-2 | Gris/CMCAB/Tween | 10.00 |
| 162-C | EX000039-056-5 | 46.714 | EX000039-053-1 | Gris/CMCAB/Tween | 9.62 |
| 163-C | EX000039-056-6 | 57.369 | EX000039-053-2 | Gris/CMCAB/Tween | 10.00 |
| 162-D | EX000039-056-7 | 50.695 | EX000039-053-1 | Gris/CMCAB/Tween | 9.62 |
| 163-D | EX000039-056-8 | 57.808 | EX000039-053-2 | Gris/CMCAB/Tween | 10.00 |
| 164-A | EX000039-057-1 | 32.734 | EX000039-053-3 | Gris/CMCAB/Tween | 9.98 |
| 165-A | EX000039-057-2 | 41.352 | EX000039-053-4 | Gris/CMCAB/SDS | 10.00 |
| 164-B | EX000039-057-3 | 32.445 | EX000039-053-3 | Gris/CMCAB/Tween | 9.98 |
| 165-B | EX000039-057-4 | 44.552 | EX000039-053-4 | Gris/CMCAB/SDS | 10.00 |
| 164-C | EX000039-057-5 | 34.460 | EX000039-053-3 | Gris/CMCAB/Tween | 9.98 |
| 165-C | EX000039-057-6 | 49.851 | EX000039-053-4 | Gris/CMCAB/SDS | 10.00 |
| 164-D | EX000039-057-7 | 35.408 | EX000039-053-3 | Gris/CMCAB/Tween | 9.98 |
| 165-D | EX000039-057-8 | 47.121 | EX000039-053-4 | Gris/CMCAB/SDS | 10.00 |
| | | | | | Calc'd |

TABLE 23A
Total Drug Released (mg)

| | Gris/CMCAB/ | Gris/CMCAB/ | Gris/CMCAB/ | Gris/CMCAB/ |
|---|---|---|---|---|
| | Tween SD | Tween SD | Tween SD | Tween SD |
| | Sample # | | | |
| Time | EX000039-053-1 | EX000039-053-2 | EX000039-053-1 | EX000039-053-2 |
| min | EX000039-056-1 | EX000039-056-2 | EX000039-056-3 | EX000039-056-4 |
| 15 | 13.5282 | 11.6295 | 13.4174 | 11.2270 |
| 30 | 20.2876 | 20.4718 | 20.7217 | 24.0079 |
| 45 | 23.1192 | 23.7565 | 23.6875 | 28.0878 |
| 60 | 24.8704 | 25.5940 | 25.4174 | 29.5686 |
| 90 | 26.9217 | 27.4981 | 27.4357 | 29.2619 |
| 120 | 27.6194 | 28.8126 | 28.2747 | 25.1265 |
| 150 | 28.4264 | 29.6012 | 28.1467 | 26.3081 |
| 180 | 29.6174 | 30.6760 | 30.9669 | 26.5987 |
| 210 | 29.8564 | 30.9146 | 31.1874 | 29.0113 |
| 240 | 29.8848 | 31.1328 | 31.6563 | 29.2049 |
| | Gris/CMCAB/ | Gris/CMCAB/ | Gris/CMCAB/ | Gris/CMCAB/ |
| | Tween SD | Tween SD | Tween SD | Tween SD |
| | Sample # | | | |
| Time | EX000039-053-1 | EX000039-053-2 | EX000039-053-1 | EX000039-053-2 |
| min | EX000039-056-5 | EX000039-056-6 | EX000039-056-7 | EX000039-056-8 |
| 15 | 14.8864 | 11.6894 | 12.6909 | 15.4677 |
| 30 | 21.0922 | 22.5678 | 20.4484 | 24.7671 |
| 45 | 23.8655 | 26.4418 | 23.2612 | 28.3437 |
| 60 | 25.5763 | 28.2613 | 25.2804 | 29.7099 |
| 90 | 27.7216 | 29.1113 | 27.3016 | 30.4436 |
| 120 | 29.5277 | 27.0263 | 28.9519 | 30.0708 |
| 150 | 30.4629 | 26.7319 | 29.3934 | 31.2110 |
| 180 | 31.1752 | 27.0181 | 30.5373 | 30.3273 |
| 210 | 31.7381 | 27.9012 | 30.7444 | 30.9532 |
| 240 | 31.6190 | 30.5104 | 31.3978 | 32.6128 |

TABLE 23B
% Drug Released

| | Gris/CMCAB/ | Gris/CMCAB/ | Gris/CMCAB/ | Gris/CMCAB/ |
|---|---|---|---|---|
| | Tween SD | Tween SD | Tween SD | Tween SD |
| | Sample # | | | |
| Time | EX000039-053-1 | EX000039-053-2 | EX000039-053-1 | EX000039-053-2 |
| min | EX000039-056-1 | EX000039-056-2 | EX000039-056-3 | EX000039-056-4 |
| 15 | 26.5698 | 22.7223 | 17.9859 | 18.3218 |
| 30 | 39.8454 | 39.9990 | 27.7772 | 39.1792 |
| 45 | 45.4067 | 46.4168 | 31.7528 | 45.8375 |
| 60 | 48.8462 | 50.0070 | 34.0718 | 48.2539 |
| 90 | 52.8749 | 53.7273 | 36.7773 | 47.7534 |
| 120 | 54.2454 | 56.2956 | 37.9020 | 41.0048 |
| 150 | 55.8303 | 57.8365 | 37.7304 | 42.9330 |
| 180 | 58.1693 | 59.9364 | 41.5108 | 43.4073 |
| 210 | 58.6388 | 60.4026 | 41.8064 | 47.3445 |
| 240 | 58.6946 | 60.8290 | 42.4350 | 47.6604 |
| | Gris/CMCAB/ | Gris/CMCAB/ | Gris/CMCAB/ | Gris/CMCAB/ |
| | Tween SD | Tween SD | Tween SD | Tween SD |
| | Sample # | | | |
| Time | EX000039-053-1 | EX000039-053-2 | EX000039-053-1 | EX000039-053-2 |
| min | EX000039-056-5 | EX000039-056-6 | EX000039-056-7 | EX000039-056-8 |
| 15 | 31.8674 | 20.3760 | 21.9534 | 30.5114 |
| 30 | 45.1521 | 39.3383 | 35.3727 | 48.8554 |
| 45 | 51.0888 | 46.0912 | 40.2385 | 55.9108 |
| 60 | 54.7512 | 49.2627 | 43.7314 | 58.6056 |
| 90 | 59.3436 | 50.7444 | 47.2277 | 60.0529 |
| 120 | 63.2100 | 47.1100 | 50.0825 | 59.3175 |
| 150 | 65.2120 | 46.5967 | 50.8462 | 61.5668 |
| 180 | 66.7369 | 47.0957 | 52.8250 | 59.8234 |
| 210 | 67.9417 | 48.6350 | 53.1834 | 61.0582 |
| 240 | 67.6868 | 53.1832 | 54.3137 | 64.3318 |

TABLE 23C
Total Drug Released (mg)

| | Gris/CMCAB/ | | Gris/CMCAB/ | |
|---|---|---|---|---|
| | Tween SD | Gris/CMCAB/SDS SD | Tween SD | Gris/CMCAB/SDS SD |
| | Sample # | | | |
| Time | EX000039-053-3 | EX000039-053-4 | EX000039-053-3 | EX000039-053-4 |
| min | EX000039-057-1 | EX000039-057-2 | EX000039-057-3 | EX000039-057-4 |
| 15 | 6.6847 | 12.3840 | 6.4044 | 12.2735 |
| 30 | 9.9322 | 15.7208 | 10.3247 | 16.5435 |
| 45 | 11.7462 | 17.7770 | 12.2167 | 18.6814 |
| 60 | 13.0593 | 19.2732 | 13.5940 | 20.2416 |
| 90 | 14.9446 | 21.5211 | 15.4063 | 22.3824 |
| 120 | 15.7383 | 22.4183 | 16.3355 | 23.4678 |
| 150 | 16.5373 | 23.2805 | 17.1367 | 24.6385 |
| 180 | 17.0484 | 24.0800 | 18.4179 | 25.4343 |
| 210 | 17.7112 | 24.5786 | 18.5572 | 25.9478 |
| 240 | | 25.0350 | 19.1823 | 26.8323 |
| | Gris/CMCAB/ | | Gris/CMCAB/ | |
| | Tween SD | Gris/CMCAB/SDS SD | Tween SD | Gris/CMCAB/SDS SD |
| | Sample # | | | |
| Time | EX000039-053-3 | EX000039-053-4 | EX000039-053-3 | EX000039-053-4 |
| min | EX000039-057-5 | EX000039-057-6 | EX000039-057-7 | EX000039-057-8 |
| 15 | 7.7630 | 13.1552 | 7.1028 | 14.2510 |
| 30 | 11.3405 | 17.5700 | 11.0540 | 18.2056 |
| 45 | 13.5710 | 19.8553 | 12.9926 | 20.6523 |
| 60 | 15.2204 | 21.5680 | 14.4580 | 22.4551 |
| 90 | 17.1880 | 23.3019 | 15.8800 | 24.5912 |
| 120 | 18.3349 | 24.9694 | 17.0413 | 25.7224 |
| 150 | 19.2422 | 26.0070 | 17.7741 | 26.6282 |
| 180 | 20.1335 | 27.1555 | 18.3948 | 27.3578 |
| 210 | 20.5399 | 27.5006 | 18.9397 | 28.1546 |
| 240 | 20.9136 | | 19.6982 | 28.8091 |

TABLE 23D
% Drug Released

| | Gris/CMCAB/ | | Gris/CMCAB/ | |
|---|---|---|---|---|
| | Tween SD | Gris/CMCAB/SDS SD | Tween SD | Gris/CMCAB/SDS SD |
| | Sample # | | | |
| Time | EX000039-053-3 | EX000039-053-4 | EX000039-053-3 | EX000039-053-4 |
| min | EX000039-057-1 | EX000039-057-2 | EX000039-057-3 | EX000039-057-4 |
| 15 | 20.4213 | 29.9476 | 19.7396 | 27.5489 |
| 30 | 30.3422 | 38.0167 | 31.8226 | 37.1334 |
| 45 | 35.8839 | 42.9893 | 37.6541 | 41.9321 |
| 60 | 39.8955 | 46.6074 | 41.8991 | 45.4339 |
| 90 | 45.6550 | 52.0435 | 47.4851 | 50.2392 |
| 120 | 48.0796 | 54.2131 | 50.3490 | 52.6756 |
| 150 | 50.5203 | 56.2980 | 52.8186 | 55.3033 |
| 180 | 52.0820 | 58.2315 | 56.7674 | 57.0895 |
| 210 | 54.1065 | 59.4371 | 57.1966 | 58.2420 |
| 240 | | 60.5409 | 59.1233 | 60.2274 |
| | Gris/CMCAB/ | | Gris/CMCAB/ | |
| | Tween SD | Gris/CMCAB/SDS SD | Tween SD | Gris/CMCAB/SDS SD |
| | Sample # | | | |
| Time | EX000039-053-3 | EX000039-053-4 | EX000039-053-3 | EX000039-053-4 |
| min | EX000039-057-5 | EX000039-057-6 | EX000039-057-7 | EX000039-057-8 |
| 15 | 22.5276 | 26.3893 | 20.0600 | 30.2432 |
| 30 | 32.9094 | 35.2453 | 31.2193 | 38.6358 |
| 45 | 39.3821 | 39.8296 | 36.6943 | 43.8280 |
| 60 | 44.1685 | 43.2652 | 40.8330 | 47.6540 |
| 90 | 49.8783 | 46.7434 | 44.8491 | 52.1872 |
| 120 | 53.2067 | 50.0885 | 48.1288 | 54.5878 |
| 150 | 55.8394 | 52.1699 | 50.1984 | 56.5101 |
| 180 | 58.4259 | 54.4738 | 51.9515 | 58.0585 |
| 210 | 59.6054 | 55.1659 | 53.4906 | 59.7495 |
| 240 | 60.6898 | | 55.6326 | 61.1384 |

TABLE 23E
Average % Released

| | Gris/CMCAB/ | Gris/CMCAB/ | Gris/CMCAB/ | Gris/CMCAB/ |
|---|---|---|---|---|
| | Tween SD | Tween SD | Tween SD | SDS SD |
| Time | Sample # | | | |
| min | EX000039-053-1 | EX000039-053-2 | EX000039-053-3 | EX000039-053-4 |
| 15 | 24.5941 | 22.9829 | 20.6871 | 28.5322 |
| 30 | 37.0368 | 41.8430 | 31.5734 | 37.2578 |
| 45 | 42.1217 | 48.5641 | 37.4036 | 42.1447 |
| 60 | 45.3501 | 51.5323 | 41.6990 | 45.7402 |
| 90 | 49.0559 | 53.0695 | 46.9669 | 50.3033 |
| 120 | 51.3600 | 50.9320 | 49.9410 | 52.8912 |
| 150 | 52.4047 | 52.2333 | 52.3442 | 55.0703 |
| 180 | 54.8105 | 52.5657 | 54.8067 | 56.9633 |
| 210 | 55.3926 | 54.3601 | 56.0998 | 58.1486 |
| 240 | 55.7825 | 56.5011 | 58.4819 | 60.6355 |

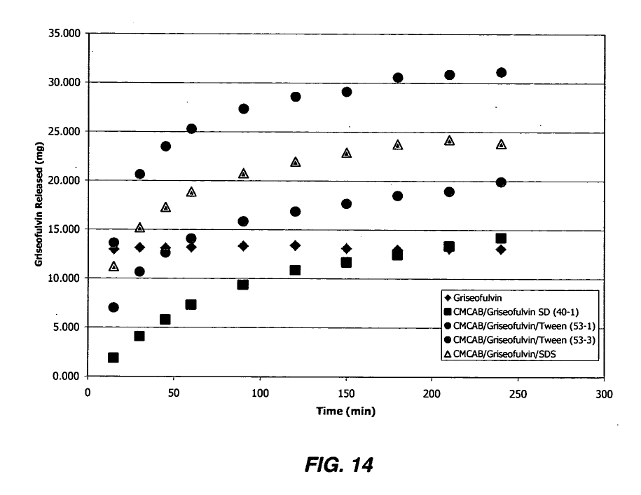

The impact of the addition of surfactants to the release profile of griseofulvin/CMCAB solid dispersions can be seen graphically in FIG. 14. The addition of surfacants, more specifically Tween 80 and SDS, to the solid dispersion compositions at levels as low as 1-5% total weight percent changed the nature of the release profile of griseofulvin. In the addition of about 1 to about 5 weight percent Tween 80 of SDS to griseofulvin/CMCAB solid dispersions, the surfactant was added prior to the co-evaporation process, and changed the near zero-order release profile of a griseofulvin/CMCAB solid dispersion into a much faster release profile. The addition of the surfactant to the griseofulvin/CMCAB solid dispersion also increased the total amount of the drug released in the system when compared to the drug alone and the griseofulvin/CMCAB solid dispersion without the surfactant.

### Example 165

This Example describes the impact of polymer and plasticizer levels.

Without wishing to be bound by any theory, the use of a plasticizer that is mutually compatible with both the drug substance and the polymeric support in a solid dispersion may reduce the level of crystallinity of the drug substance trapped in the solid dispersion by generating a system of compatible ingredients and reducing the likelihood of drug substance “pooling” that would ultimately result in crystallization of the drug substance within the solid dispersion. To evaluate this theory, the impact of various plasticizers and plasticizer levels on the % crystallinity of a solid dispersion containing C-A-P or CMCAB and ibuprofen was investigated. These experiments indicated an impact of plasticizer (max loading of 10%) on the percent crystallinity of solid dispersions of ibuprofen with CMCAB or C-A-P as the polymeric carrier, as indicated in Table 24.

TABLE 24

| | | | | | | | X Ray | | | | | | | | |
|---|---|---|---|---|---|---|---|---|---|---|---|---|---|---|---|
| | | | | | | | wt % | % | | | | Mod. DSC | | | |
| | CMCAB | IB | Pz | CMCAB | | | Crystaline | Amorphous | LC wt % | | | Tg | Tm | Tg | Tm |
| Sample # | (g) | (g) | (g) | % | IB % | Pz % | IB | IB | IB | CMCAB | Pz | (1st) | (1st) | (2nd) | (2nd) |
| X-29555-17-1 | 4.19 | 1.397 | 0.45 | 69.41 | 23.14 | 7.45 | 6.1 | 16.8 | 22.9 | 72.5 | 4.6 | nd | 69.3 | 76.7 | nd |
| X-29555-17-2 | 6.02 | 0.074 | 0 | 98.79 | 1.21 | 0.00 | 0 | 1.4 | 1.4 | 98.6 | 0 | 132 | nd | nd | 130 |
| X-29555-17-3 | 5.24 | 0.513 | 0.31 | 86.43 | 8.46 | 5.11 | 2.1 | 12.1 | 6.3 | 90.1 | 3.6 | nd | 75.2 | 93.7 | nd |
| X-29555-17-4 | 5.078 | 0.95 | 0 | 84.24 | 15.76 | 0.00 | 0 | 6.3 | 14.2 | 85.8 | 0 | 110 | nd | 109 | nd |
| X-29555-17-5 | 4.77 | 0.922 | 0.3 | 79.61 | 15.39 | 5.01 | 0 | 12.1 | 12.1 | 84.5 | 3.4 | 96.1 | nd | 93.9 | nd |
| X-29555-17-6 | 5.504 | 0.08 | 0.6 | 89.00 | 1.29 | 9.70 | 0 | 1.3 | 1.3 | 92.3 | 6.4 | 116 | nd | 109 | nd |
| X-29555-17-7 | 5.99 | 0.11 | 0 | 98.20 | 1.80 | 0.00 | 0 | 1.8 | 1.8 | 98.2 | 0 | 128 | nd | 128 | nd |
| X-29555-17-8 | 3.62 | 1.8 | 0.598 | 60.15 | 29.91 | 9.94 | 8.4 | 22.7 | 31.1 | 62.5 | 6.4 | nd | 69.6 | 61 | nd |
| X-29555-17-9 | 4.55 | 0.937 | 0.65 | 74.14 | 15.27 | 10.59 | 2.9 | 12.5 | 15.4 | 78.6 | 6 | nd | 66.7 | 76.8 | nd |
| X-29555-17-10 | 4.21 | 1.82 | 0 | 69.82 | 30.18 | 0.00 | 7.9 | 23.1 | 31 | 69 | 0 | nd | 73.4 | 58.1 | nd |
| X-29555-17-11 | 4.2 | 1.8 | 0 | 70.00 | 30.00 | 0.00 | 8.3 | 21.9 | 30.2 | 69.8 | 0 | nd | 74 | 62.7 | nd |
| X-29555-17-12 | 5.34 | 0.08 | 0.58 | 89.00 | 1.33 | 9.67 | 0 | 1.9 | 1.9 | 91.6 | 6.5 | 112 | nd | 111 | nd |
| X-29555-17-13 | 3.6 | 1.83 | 0.6 | 59.70 | 30.35 | 9.95 | 9.6 | 22.2 | 31.8 | 61.3 | 6.9 | nd | 67.7 | 56.1 | nd |
| X-29555-17-14 | 4.51 | 1.43 | 0.151 | 74.04 | 23.48 | 2.48 | 6.7 | 16.2 | 22.9 | 75.2 | 1.9 | 62.6 | 72.3 | 74.8 | nd |

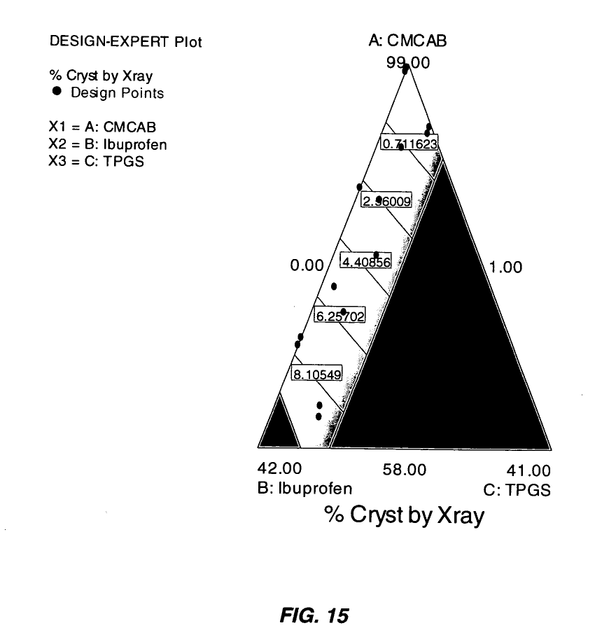

FIG. 15 shows the impact of TPGS on % crystallinity of ibuprofen/CMCAB solid dispersions (D-Optimal Mixture DOE Results).

### Example 166

This Example describes the impact of sample preparation method. Solid dispersions were prepared using co-precipitation methods in which the drug, enteric polymer, and additives were dissolved in acetone then precipitated by adding the mixture to water. The poor water solubility of the drug results in the drug co-precipitating with the enteric cellulosic to produce a solid dispersion. Evaluated here are a series of strategies for preparing solid dispersions, including co-precipitation (flake ppt'n and powder ppt'n), co-evaporation, and spray drying and determined the impact the various methods had on the % crystallinity of the solid dispersion, as indicated in Table 25, which shows the impact of method of preparation on % crystallinity of solid dispersions.

TABLE 25

| | | | | wt % | wt % | | | | | |
|---|---|---|---|---|---|---|---|---|---|---|
| Preparation | | | | Crystalline | Amorphous | wt % | Tg | Tm | Tg | Tm |
| Method | Sample # | CAP % | IB % | IB | IB | IB | (1st) | (1st) | (2nd) | (2nd) |
| Spray | X-29555-27A-1 | 83.00 | 17.00 | 0 | 17.5 | 17.5 | 120.6 | 74.4 | 93.0 | nd |
| | X-29555-27A-2 | 60.00 | 40.00 | 5.1 | 29.9 | 35 | nd | 75 | 99.68 | 70.73 |
| | X-29555-27A-3 | 40.00 | 60.00 | 12.1 | 35.5 | 47.6 | 135.1 | 75 | 99.67 | 70.73 |
| | X-29555-27A-4 | 33.00 | 67.00 | 17.2 | 37 | 54.2 | nd | 74.9 | 96.64 | 70.29 |
| | X-29555-27A-5 | 5.00 | 95.00 | 19.4 | 39.1 | 58.5 | nd | 74.9 | 101.4 | 71.2 |
| Co-Evp | X-29555-27B-1 | 83.00 | 17.00 | * | * | 15.8 | nd | 74.4 | 97.27 | nd |
| | X-29555-27B-2 | 60.00 | 40.00 | 12.3 | 22.7 | 35 | nd | 74.1 | 101.8 | 71.28 |
| | X-29555-27B-3 | 40.00 | 60.00 | 22 | 29.8 | 51.8 | nd | 75.7 | 99.09 | 71.2 |
| | X-29555-27B-4 | 33.00 | 67.00 | ** | ** | 61.6 | nd | 75.7 | 99.4 | 71.1 |
| | X-29555-27B-5 | 5.00 | 95.00 | ** | ** | 92.9 | nd | 75.4 | nd | 162.6 |
| Co-Ppt | X-29555-27C-1 | 83.00 | 17.00 | 1.6 | 16.6 | 18.2 | nd | 75.7 | 139.1 | nd |
| | X-29555-27C-2 | 60.00 | 40.00 | 4.1 | 35.5 | 39.6 | nd | 75.8 | 105.4 | 71.89 |
| | X-29555-27C-3 | 40.00 | 60.00 | 12.6 | 46.4 | 59 | nd | 72.1 | 100.6 | 71.3 |
| | X-29555-27C-4 | 33.00 | 67.00 | 16.4 | 49.2 | 65.6 | nd | 75 | 105.4 | 71.43 |
| | X-29555-27C-5 | 5.00 | 95.00 | ** | ** | 94.8 | hd | 74.5 | nd | nd |
| Powder Ppt | X-29555-27D-1 | 83.00 | 17.00 | * | * | 16.4 | 120.6 | 74.7 | 111.7 | nd |
| | X-29555-27D-2 | 60.00 | 40.00 | 6.3 | 23.6 | 29.9 | 123.8 | 74.8 | 102.9 | nd |
| | X-29555-27D-3 | 40.00 | 60.00 | 40.9 | 18.9 | 59.8 | nd | 75.2 | 104.1 | 70.9 |
| | X-29555-27D-4 | 33.00 | 67.00 | 32.2 | 32.1 | 64.3 | nd | 75.1 | 106.5 | 71.75 |
| | X-29555-27D-5 | 5.00 | 95.00 | ** | ** | 92.9 | nd | 72.8 | nd | 72.16 |

**Indicates the sample morphology was out of the calibrated range for X-ray

### Example 167

This Example describes the impact of process parameters (Temperature). Specifically, the impact of drying temperatures between 40 and 100° C. on the % crystallinity of solid dispersions was evaluated. Increased drying temperatures or processing temperatures can reduce the crystallinity of a solid dispersion prepared by co-precipitation, as indicated in Table 26, which shows the impact of drying temperature on % crystallinity of ibuprofen/C-A-P solid dispersions.

TABLE 26

| | | | | | | | | X-ray | | | | |
|---|---|---|---|---|---|---|---|---|---|---|---|---|
| | Dry | | | | | | | Wt. % | | | | |
| | Temp. | | | | | | | Cryst. | Wt % | LCWt % | | |
| Sample # | Deg. C | C-A-P(g) | IB(g) | Pz(g) | C-A-P % | IB % | Pz % | IB | Amor* | IB | C-A-P* | Pz |
| X-29555-26-1 | 40 | 9.72 | 2.4 | 0 | 80.20 | 19.80 | 0.00 | 3 | 17.6 | 20.6 | 79.4 | 0 |
| X-29555-26-2 | 60 | 9.72 | 2.4 | 0 | 80.20 | 19.80 | 0.00 | 0.9 | 16.1 | 17 | 83 | 0 |
| X-29555-26-3 | 80 | 9.72 | 2.4 | 0 | 80.20 | 19.80 | 0.00 | 0 | 12.9 | 12.9 | 87.1 | 0 |
| X-29555-26-4 | 100 | 9.72 | 2.4 | 0 | 80.20 | 19.80 | 0.00 | 0 | 4.5 | 4.5 | 95.5 | 0 |
| X-29555-26-5 | 40 | 7.29 | 4.8 | 0 | 60.30 | 39.70 | 0.00 | 8.5 | 31.9 | 40.4 | 59.6 | 0 |
| X-29555-26-6 | 60 | 7.29 | 4.8 | 0 | 60.30 | 39.70 | 0.00 | 6 | 27.2 | 33.2 | 66.8 | 0 |
| X-29555-26-7 | 80 | 7.29 | 4.8 | 0 | 60.30 | 39.70 | 0.00 | 0 | 24.4 | 24.4 | 75.6 | 0 |
| X-29555-26-8 | 100 | 7.29 | 4.8 | 0 | 60.30 | 39.70 | 0.00 | NA | NA | 18.5 | 81.5 | 0 |
| X-29555-26-9 | 40 | 4.81 | 7.2 | 0 | 40.05 | 59.95 | 0.00 | 22.2 | 40.4 | 62.6 | 37.4 | 0 |
| X-29555-26-10 | 60 | 4.81 | 7.2 | 0 | 40.05 | 59.95 | 0.00 | 20.3 | 30.9 | 51.2 | 48.8 | 0 |
| X-29555-26-11 | 80 | 4.81 | 7.2 | 0 | 40.05 | 59.95 | 0.00 | 5.3 | 13.2 | 18.5 | 81.5 | 0 |
| X-29555-26-12 | 100 | 4.81 | 7.2 | 0 | 40.05 | 59.95 | 0.00 | NA | NA | 20 | 80 | 0 |
| X-29555-26-13 | 40 | 2.4 | 9.6 | 0 | 20.00 | 80.00 | 0.00 | 33.8 | 48.7 | 82.5 | 17.5 | 0 |
| X-29555-26-14 | 60 | 2.4 | 9.6 | 0 | 20.00 | 80.00 | 0.00 | 40.5 | 42.4 | 82.9 | 17.1 | 0 |
| X-29555-26-15 | 80 | 2.4 | 9.6 | 0 | 20.00 | 80.00 | 0.00 | 13.1 | 42 | 55.1 | 44.9 | 0 |
| X-29555-26-16 | 100 | 2.4 | 9.6 | 0 | 20.00 | 80.00 | 0.00 | NA | NA | 18.6 | 81.4 | 0 |
| X-29555-26-17 | 40 | 0.6 | 11.4 | 0 | 5.00 | 95.00 | 0.00 | 46.9 | 52.3 | 99.2 | 0.8 | 0 |
| X-29555-26-18 | 60 | 0.6 | 11.4 | 0 | 5.00 | 95.00 | 0.00 | 102.6 | -3.7 | 98.9 | 1.1 | 0 |
| X-29555-26-19 | 80 | 0.6 | 11.4 | 0 | 5.00 | 95.00 | 0.00 | NA | NA | 92.9 | 7.1 | 0 |
| X-29555-26-20 | 100 | 0.6 | 11.4 | 0 | 5.00 | 95.00 | 0.00 | NA | NA | 10.3 | 89.7 | 0 |

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A pharmaceutical composition comprising:
at least one pharmaceutically active agent having low solubility in a medium, and
at least one carboxyalkylcellulose ester comprising an anhydroglucose repeat unit having the structure: 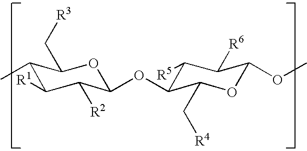
wherein:
R¹-R⁶ are each independently selected from —OH, —OC(O)(alkyl), and —O(CH₂)ₓC(O)OH, and pharmaceutically acceptable salts, wherein x ranges from 1-3,
a degree of substitution per anhydroglucose of —OH ranges from 0.1 to 0.7,
a degree of substitution per anhydroglucose of —OC(O)(alkyl) ranges from 0.1 to 2.7, and
a degree of substitution per anhydroglucose of —O(CH₂)ₓC(O)OH ranges from 0.2 to 0.75.

2. The composition according to claim 1, wherein the composition comprises a solid dispersion.

3. The composition according to claim 1, further comprising at least one additive chosen from binding agent, filling agent, lubricating agent, suspending agent, sweetener, flavoring agent, preservative, buffer, wetting agent, disintegrant, effervescent agent, or other excipient.

4. The composition according to claim 1, wherein further comprising at least one of the additive chosen from Vitamin E TPGS, sucrose acetate isobutyrate, glucose pentapropionate, diethyl phthalate, triacetin, polyoxyethylenesorbitan monooleate and sodium dodecylsulfate.

5. The composition according to claim 1, wherein the —OC(O)(alkyl) is chosen from —OC(O)(C₁-C₂₁ alkyl).

6. The composition according to claim 1, wherein the —OC(O)(alkyl) is chosen from —OC(O)(C₁-C₁₁ alkyl).

7. The composition according to claim 1, wherein the —OC(O)(alkyl) is chosen from —OC(O)(C₁-C₅ alkyl).

8. The composition according to claim 1, wherein the —OC(O)(alkyl) is chosen from —OC(O)(C₁-C₃ alkyl).

9. The composition according to claim 1, wherein the at least one carboxyalkylcellulose ester is chosen from carboxymethylcellulose esters having a degree of substitution ranging from 0.2 to 0.4.

10. The composition according to claim 9, wherein the carboxyalkylcellulose ester is carboxymethylcellulose acetate having a degree of substitution per anhydroglucose of —OC(O)CH₃ ranging from 1.5 to 2.7.

11. The composition according to claim 9, wherein the at least one carboxyalkylcellulose ester is carboxymethylcellulose propionate having a degree of substitution per anhydroglucose of —OC(O)CH₂CH₃ ranging from 1.5 to 2.7.

12. The composition according to claim 9, wherein the at least one carboxyalkylcellulose ester is carboxymethylcellulose butyrate having a degree of substitution per anhydroglucose of —OC(O)CH₂CH₂CH₃ ranging from 1.5 to 2.7.

13. The composition according to claim 9, wherein the at least one carboxyalkylcellulose ester is carboxymethylcellulose acetate propionate having a degree of substitution per anhydroglucose of —OC(O)CH₃ ranging from 0.1 to 2.65 and a degree of substitution per anhydroglucose of —OC(O)CH₂CH₂H₃ ranging from 0.1 to 2.6.

14. The composition according to claim 9, wherein the at least one carboxyalkylcellulose ester is carboxymethylcellulose acetate butyrate having a degree of substitution per anhydroglucose of —OC(O)CH₃ ranging from 0.1 to 1.65 and a degree of substitution per anhydroglucose of —OC(O)CH₂CH₂H₃ ranging from 0.1 to 2.6.

15. The composition according to claim 1, wherein the composition comprises a polymeric blend.

16. The composition according to claim 1, wherein in pharmaceutically acceptable media, the composition exhibits release of the pharmaceutically active agent at a pH of at least 5.

17. The composition according to claim 1, wherein in pharmaceutically acceptable media, the composition exhibits release of the pharmaceutically active agent at a pH of at least 6.

18. The composition according to claim 1, wherein in pharmaceutically acceptable media, the composition exhibits release of the pharmaceutically active agent at a pH of at least 6.5.

19. A composition comprising:
at least one pharmaceutically active agent, and
at least one carboxyalkylcellulose ester comprising an anhydroglucose repeat unit having the structure: 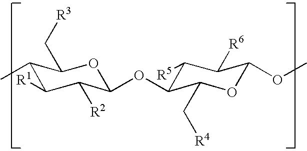
wherein:
R¹-R⁶ are each independently selected from —OH, —OC(O)(alkyl), and —O(CH₂)ₓC(O)OH, and pharmaceutically acceptable salts, wherein x ranges from 1-3,
a degree of substitution per anhydroglucose of —OH ranges from 0.1 to 0.7,
a degree of substitution per anhydroglucose of —OC(O)(alkyl) ranges from 0.1 to 2.7, and
a degree of substitution per anhydroglucose of —O(CH₂)ₓC(O)OH ranges from 0.2 to 0.75,
wherein the composition is in the form of a solid dispersion.

20. The composition according to claim 19, wherein the carboxyalkylcellulose ester is a carboxymethylcellulose acetate butyrate having an inherent viscosity of 0.35 to 0.60 dL/g.

21. The composition according to claim 19, wherein the degree of substitution per anhydroglucose of —OCH₂C(O)OH ranges from 0.751 to 1.2.

22. The composition according to claim 19, prepared by the process of co-precipitation.

23. The composition according to claim 19, prepared by the process of co-evaporation.

24. The composition according to claim 19, prepared by the process of spray drying.

25. The composition according to claim 19, prepared by the process of lyophilization.

26. The composition according to claim 19, prepared by a solvent-free process.

27. The composition according to claim 19, prepared by melt blending.

28. The composition according to claim 19, prepared by melt extrusion.

29. A pharmaceutical composition comprising:
at least one pharmaceutically active agent, wherein at least 10,000 mL of water is required to dissolve 1 g of the agent, and
at least one carboxyalkylcellulose ester comprising an anhydroglucose repeat unit having the structure: 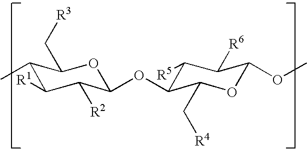
wherein:
R¹-R⁶ are each independently selected from —OH, —OC(O)(alkyl), and —O(CH₂)ₓC(O)OH, and pharmaceutically acceptable salts, wherein x ranges from 1-3,
a degree of substitution per anhydroglucose of —OH ranges from 0.1 to 0.7,
a degree of substitution per anhydroglucose of —OC(O)(alkyl) ranges from 0.1 to 2.7, and
a degree of substitution per anhydroglucose of —O(CH₂)ₓC(O)OH ranges from 0.2 to 0.75.

30. A method of treating a mammal in need thereof with a pharmaceutical composition, comprising:
administering to the mammal in need of treatment the pharmaceutical composition comprising:
a therapeutically effective amount of at least one poorly soluble pharmaceutically active agent, and
at least one carboxyalkylcellulose ester comprising an anhydroglucose repeat unit having the structure: 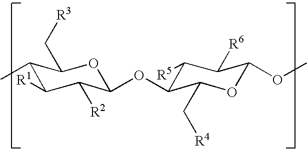
wherein:
R¹-R⁶ are each independently selected from —OH, —O—C(O)(alkyl), and —O(CH₂)ₓC(O)OH, and pharmaceutically acceptable salts, wherein x ranges from 1-3,
a degree of substitution per anhydroglucose of —OH ranges from 0.1 to 0.7,
a degree of substitution per anhydroglucose of —OC(O)(alkyl) ranges from 0.1 to 2.7, and
a degree of substitution per anhydroglucose of —O(CH₂)ₓC(O)OH ranges from 0.2to 0.75.

31. A pharmaceutical composition comprising:
at least one pharmaceutically active agent having low solubility in a medium, and
at least one carboxyalkylcellulose ester comprising an anhydroglucose repeat unit having the structure: 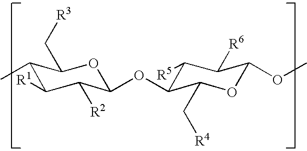
wherein:
R¹-R⁶ are each independently selected from —OH, —OC(O)(alkyl), and —O(CH₂)ₓC(O)OH, O⁻A⁺, and —O(CH₂)ₓC(O)O⁻A⁺, wherein x ranges from 1-3, and A⁺ is a counter ion,
a degree of substitution per anhydroglucose of —OH and O⁻A⁺ ranges from 0.1 to 0.7,
a degree of substitution per anhydroglucose of —OC(O)(alkyl) ranges from 0.1 to 2.7, and
a degree of substitution per anhydroglucose of —O(CH₂)ₓC(O)OH and —O(CH₂)ₓ(O)O⁻A⁺ ranges from 0.2 to 0.75.

32. The composition according to claim 31, wherein the composition comprises a solid dispersion.

33. The composition according to claim 31, wherein each A⁺ is independently selected from monovalent inorganic cations, divalent inorganic cations, ammonium salts, and alkyl ammonium salts.

34. The composition according to claim 33, wherein the monovalent inorganic cations are chosen from lithium, sodium, potassium, rubidium, cesium, and silver.

35. The composition according to claim 33, wherein the divalent inorganic cations are chosen from magnesium, calcium, nickel, zinc, iron copper, and manganese.
